(19) United States
(12) Patent Application Publication
(10) Pub. No.: US 2024/0033727 A1
(43) Pub. Date: Feb. 1, 2024

(54) REAGENT EXCHANGE METHODS, DEVICES, AND SYSTEMS

(71) Applicant: SUZHOU SINGLERON BIOTECHNOLOGIES CO., LTD., Suzhou (CN)

(72) Inventors: Yunyan QIU, Suzhou (CN); Dongdong CHU, Suzhou (CN); Huanlin CHEN, Suzhou (CN); Jian MIAO, Suzhou (CN)

(21) Appl. No.: 18/255,569

(22) PCT Filed: Dec. 2, 2021
(86) PCT No.: PCT/CN2021/135159

§ 371 Date: Jun. 1, 2023

(30) Foreign Application Priority Data
Dec. 2, 2020 (CN) 202011399906.7
May 17, 2021 (CN) 202110533968.0
May 17, 2021 (CN) 202110534947.0
May 17, 2021 (CN) 202110535870.9

(51) Int. Cl. B01L 3/00

(52) U.S. Cl. CPC B01L 3/502707; B01L 3/502715; B01L 2200/16; B01L 2300/08

(57) ABSTRACT

Provided include a microfluidic device capable of performing reagent exchange, a gas-flow control device, a cell reaction module, and a device for preparing cell samples, such as a single-cell sample, and method of use thereof.

### CROSS-REFERENCE TO RELATED APPLICATIONS

This application is a U.S. national phase application under 35 U.S.C. § 371 of International Application No. PCT/CN2021/135159, filed on Dec. 2, 2021 and published as WO 2022/117053 A1 on Jun. 9, 2022, which claims the benefit of priority to Chinese Patent Application No. 202011399906.7, filed on Dec. 2, 2020, Chinese Patent Application No. 202110534947.0, filed on May 17, 2021, Chinese Patent Application No. 202110533968.0, filed on May 17, 2021, and Chinese Patent Application No. 202110535870.9, filed on May 17, 2021, the content of each of these related applications is incorporated herein by reference in its entirety.

### BACKGROUND

### Field

The present disclosure relates generally to the fields of bioengineering in particular to reagent loading, sequencing, sample preparation, or sample analysis, such as high-throughput single-cell sample preparations and analysis.

### Description of the Related Art

Current methods of sample loading and analysis can be imprecise, which can result in accurate results. There is a need for improved methods of sample loading and analysis that can generate accurate results.

### SUMMARY

Provided include a microfluidic device capable of performing reagent exchange, a gas-flow control device, a cell reaction module, a device for preparing cell samples, such as a single-cell sample, and method of use thereof.

The present disclosure provides a microfluidic device capable of performing reagent exchange as well as a method of use and an application thereof. In the microfluidic device (e.g., a microfluidic chip illustrated in FIGS. 1A-1B), a reagent exchange process can be performed on the device without the need to externally inject reagents during use, thereby improving the operability of the microfluidic device and ensuring the accuracy of experimental results.

The present disclosure provides a microfluidic device capable of performing reagent exchange, comprising a reagent exchange unit and a reaction (e.g., working unit 108, FIG. 1B) bonded to each other, wherein an upper surface of the reagent exchange unit comprises reagent reservoirs, a product reservoir, a waste liquid reservoir (or a waste reservoir), and a reagent exchange reservoir, and the reagent reservoirs are connected to the reagent exchange reservoir through microchannels; a lower surface of the reagent exchange unit comprises a recess connected to the reagent exchange reservoir, the product reservoir, and the waste liquid reservoir through microchannels; and the working unit covers the microchannels and the recess, and forms, together with the recess, a reaction chamber (e.g., working section 106, FIG. 1B) of the microfluidic chip.

The microfluidic chip provided herein combines a reagent exchange process and a reaction process to enable exchange and collection of different liquids on the microfluidic chip and ensure that each liquid can sufficiently enter the microfluidic chip for reaction. The special structural design increases the resolution of the operation process and the precision of liquid control, and avoids problems such as bubbles, waste liquids, and contamination that may occur during operation. In addition, the structural design of the present microfluidic device can simplify the processing process of the microfluidic chip, and combine different structures to improve the stability of chip processing.

The microchannel design used in the present microfluidic device can be laid out according to the actual situation to realize the exchange of a plurality of reagents. The reagent exchange reservoir can avoid the formation of bubbles during the transfer of reagents. The plurality of needed reagents can also be uniformly mixed in the reagent exchange reservoir, and the volume of redundant liquids can be reduced. More convenient and precise use is achieved without the aid of precise instruments, and large errors will not be caused during the experiment even in the first use by an experimenter. Further, the working unit is configured according to a reaction being performed. For example, in the case of preparation of a single-cell sequencing sample, the working unit has the function of capturing a single cell.

As an optional technical solution of the present microfluidic device, the upper surface of the reagent exchange unit is divided into a left functional area and a right functional area, the left functional area comprises the product reservoir and the waste liquid reservoir, and the right functional area comprises at least two reagent reservoirs.

Optionally, the reagent exchange reservoir is disposed in the right functional area.

As an optional technical solution of the present microfluidic device, cross-sectional shapes of the product reservoir and the waste liquid reservoir are the same or different.

Optionally, the cross-sectional shape of the product reservoir is a rectangle.

The height and volume of the product reservoir may be set according to requirements. For example, the height of the product reservoir may be set to 5 to 20 mm (for example, 5 mm, 6 mm, 8 mm, 10 mm, 12 mm, 15 mm, 18 mm, or 20 mm), and the volume may be set to 0.1 to 10 mL (for example, 0.1 mL, 0.5 mL, 1 mL, 2 mL, 3 mL, 5 mL, 6 mL, 8 mL, or 10 mL).

Optionally, the cross-sectional shape of the waste reservoir is a circle or an ellipse.

The height and volume of the waste reservoir may also be set according to requirements. For example, the height of the waste liquid reservoir may be set to 5 to 20 mm, and the volume may be set to 0.1 to 10 mL.

As an optional technical solution of the present microfluidic device, cross-sectional shapes of the reagent reservoirs are any one or a combination of at least two of a circle, a rectangle, an ellipse, a semicircle, or a trapezoid.

Since there are many types of reagents during the experiment, a large number of reagent reservoirs need to be designed. For ease of differentiation, the type of the reagent filled in each reagent reservoir may be identified according to the number, volumes, or shapes of the reagent reservoirs. In the present microfluidic device, the number, volumes, and shapes of the reagent reservoirs may be selected according to actual requirements. For example, the heights of the reagent reservoirs may be 0.1 to 10 mm, and the volumes may be 0.1 to 5 mL.

As an optional technical solution of the present microfluidic device, the cross-sectional shape of the reagent exchange reservoir is any one or a combination of at least two of a circle, a rectangle, an ellipse, a semicircle, or a trapezoid.

Similarly, the height and volume of the reagent exchange reservoir in the present microfluidic device as a component for realizing reagent exchange may also be adjusted according to the actual experiment. For example, the height of the reagent exchange reservoir is 0.1 to 10 mm, and the volume is 0.1 to 10 mL.

As an optional technical solution, the present microfluidic device is any one of a rectangle, a circle, a trapezoid, or an ellipse.

Optionally, the working unit is any one of a rectangle, a circle, a trapezoid, or an ellipse.

The size of neither the microfluidic chip nor the working unit is limited, as long as the aforementioned various types of reagent reservoirs and microchannels can be accommodated.

Optionally, the microfluidic device is an integral structure.

The present disclosure also provides a method of using the microfluidic device as described herein, the method comprising: placing reaction reagents in the reagent reservoirs of the microfluidic device, and then making the reaction reagents flow into the reagent exchange reservoir through the microchannels; and transferring the reaction reagents in the reagent exchange reservoir to the reaction chamber through the microchannels to undergo a reaction, and transferring a product in the reaction chamber to the product reservoir to obtain a reaction product after the reaction is completed.

As an optional technical solution, in the present method, the reaction reagent flows into the reagent exchange reservoir by means of pressurization.

Optionally, the reaction reagents in the reagent exchange reservoir are transferred to the reaction chamber by means of depressurization or pressurization.

Optionally, the product is transferred to the product reservoir by means of depressurization.

For example, the method of using the present microfluidic device may include: placing reaction reagents in the reagent reservoirs of the microfluidic device, pressurizing the reaction reagents during a reaction, and making the reaction reagents flow into the reagent exchange reservoir through the microchannels; and depressurizing the waste liquid reservoir or the product reservoir of the microfluidic device, so that the reaction reagents in the reagent exchange reservoir flow to the reaction chamber through the microchannels to undergo a reaction, and then transferring the product in the reaction chamber to the product reservoir by depressurization to obtain the reaction product.

The present disclosure further provides an application of the microfluidic device as described herein in a reagent exchange reaction.

The present microfluidic device has a wide scope of application, and basically all reactions involving reagent exchange can be carried out using the microfluidic device. Moreover, the size of each reagent reservoir on the microfluidic device can be appropriately adjusted according to different reactions. That is, a suitable microfluidic device is prepared according to an application scenario.

Optionally, the reagent exchange reaction comprises any one of cell capture, cell membrane lysis, RNA capture, single-cell sequencing, or medication screening.

Exemplarily, the present microfluidic device may be used for single-cell sequencing, whereby different reagents can enter the working section for reaction according to different sequences, speeds, and volumes or other conditions, so as to complete a series of steps requiring exchange of different reagents such as single-cell capture, lysis, and RNA capture.

Exemplarily, the present microfluidic device may also be used for medication screening, whereby different medication reaction reagents can sequentially enter the reaction chamber to detect reactions of cell samples under different medication treatments.

The above descriptions are merely examples of the application scenario of the microfluidic device in the present disclosure. In a word, a system requiring reaction of different reagents can perform automatic operation by means of the present microfluidic device.

As compared with the prior art, the microfluidic device and uses thereof can have one or more of the following beneficial effects:
(1) in the present microfluidic device, the reagent exchange unit and the reaction unit are combined, and due to the design of the reagent exchange reservoir and the microchannels, automatic exchange of reagents is realized on the device; detection reagents of the microfluidic device are added to reagent chambers of the device in advance without the need to externally inject reagents during use, so that the accuracy of different reagents during exchange is ensured; operations such as mixing can be performed on the reaction reagents; waste of reagents is avoided; and the need for manpower and machinery is reduced:
(2) the method for preparing the present microfluidic device is simple, the bonding process of the reaction unit and the reagent exchange unit may be selected according to requirements of the reagents, or an integral disposable device (e.g. a chip) may be manufactured according to requirements; the integral disposable device can effectively avoid cross-contamination, which can not only simplify the experimental process, but also improve the accuracy of experimental results, reduce human errors during the experiment, have desirable repeatability, and improve the reliability of the experiment results.

In a second specific embodiment, the present disclosure provides a reaction module having an integrated gas path control and a method of using the same to perform a cell reaction. An integrated gas path control plate is provided with grooves therein to integrate various driving gas path channels. A plurality of driving gas path channels are connected by means of solenoid valves, so as to realize connection or separation of different driving gas path channels according to needs through a control switch of an upper computer, and a driving gas is used as a driving force for injecting reaction reagents to transport the reaction reagents in a flow path. During use, the driving gas is injected into different driving gas path channels, so that reaction reagents in different reagent reservoirs can be injected into the reaction chamber one by one. The injection processes of different reaction reagents are independent of each other and do not affect each other, so that the flow path control process of the reaction reagents is stable and reliable, and the entire cell reaction module can be mounted easily and occupies a small space.

The present disclosure provides a reaction module (such as a cell reaction module) having integrated gas path control, wherein the reaction module comprises an integrated gas path control plate (also referred to as a gas-flow control device herein) and a cell reaction plate (also referred to as a microfluidic device herein) attached to each other, at least two mutually independent driving gas path channels are provided inside the integrated gas path control plate. A surface of the cell reaction plate on a side thereof attached to the integrated gas path control plate is provided with reagent reservoirs of the same number as the driving gas path channels. Each driving gas path channel independently communicates with one reagent reservoir. A surface of the cell reaction plate on a side thereof away from the integrated gas path control plate is provided with a reaction chamber, and the reaction chamber communicates with the reagent reservoirs. Reaction reagents are injected into the reagent reservoirs in advance, a driving gas is injected into the reagent reservoirs through the driving gas path channels, and the reaction reagents in the reagent reservoirs flow into the reaction chamber under the pressure of the driving gas.

An integrated gas path control plate is provided with grooves therein to integrate various driving gas path channels. A plurality of driving gas path channels are connected by means of solenoid valves, so as to realize connection or separation of different driving gas path channels according to needs through a control switch of an upper computer. A driving gas is used as a driving force for injecting reaction reagents to transport the reaction reagents in a flow path. During use, the driving gas is injected into different driving gas path channels, so that reaction reagents in different reagent reservoirs can be injected into the reaction chamber one by one. The injection processes of different reaction reagents are independent of each other and do not affect each other, so that the flow path control process of the reaction reagents is stable and reliable, and the entire cell reaction module can be mounted easily and occupies a small space.

It should be noted that the formation manner of the driving gas path channels is not specifically required or specially defined herein, as long as smooth transportation of the driving gas is satisfied. Exemplarily, the following two solutions may be adopted:
Solution 1: the integrated gas path control plate is an integral structure, and the driving gas path channels are directly formed in the integrated gas path control plate by means of casting, drilling, additive manufacturing, or the like;
Solution 2: the integrated gas path control plate is a split structure, which is formed by laminating an upper gas path plate and a lower gas path plate. The laminating surface between the upper gas path plate and/or lower gas path plate is provided with gas path grooves, and after the upper gas path plate and the lower gas path plate are attached to each other, the gas path grooves are closed to form the driving gas path channels. It can certainly be understood that in this solution, the gas path grooves may be provided on a lower surface of the upper gas path plate or an upper surface of the lower gas path plate, or may be provided on both the lower surface of the upper gas path plate and the upper surface of the lower gas path plate.

It should be noted that the cell reaction plate is also provided with microchannels therein for reaction reagents to flow therethrough. Reference may be made to the above description of the driving gas path channels for the formation manner of the microchannels. That is, the cell reaction plate may be an integral structure or a split structure. When the cell reaction plate is an integral structure, the microchannels are directly formed in the cell reaction plate by means of casting, drilling, additive manufacturing, or the like. Certainly, the cell reaction plate may be a split structure formed by laminating an upper reaction plate and a lower reaction plate. The laminating surface of the upper reaction plate and/or lower reaction plate is provided with flow path grooves, and after the upper reaction plate and the lower reaction plate are attached to each other, the flow path grooves are closed to form microchannels.

It should be noted that the number of the reagent reservoirs is not specifically required herein. The number of the reagent reservoirs is the same as that of the driving gas path channels, and an outlet end of each driving gas path channel corresponds to one reagent reservoir. Those skilled in the art can design the number and position of the reagent reservoirs specifically according to different cell reactions.

As an optional technical solution of the present reaction module, the surface of the cell reaction plate on the side thereof attached to the integrated gas path control plate is further provided with a buffer reservoir (also referred to as a reagent exchange reservoir herein). The reagent reservoirs and the reaction chamber each independently communicate with the buffer reservoir, the driving gas path channels, the reagent reservoirs, and the buffer reservoir communicate with each other in sequence in a flow direction of the driving gas. The driving gas is fed into the reagent reservoirs through the driving gas path channels, and the reaction reagents stored in the reagent reservoirs enter the buffer reservoir one by one and are injected into the reaction chamber through the buffer reservoir.

Optionally, inlet ends of the driving gas path channels are provided with solenoid valves, and the solenoid valves are used to control a feeding amount of the driving gas.

The solenoid valves connected to the driving gas path channels are mainly used to control the flow of the driving gas. During use of the cell reaction module provided in the present invention, the reaction reagents stored in the reagent reservoirs are pressed into the buffer reservoir under the pressure of the driving gas, and the flow of the driving gas is adjusted by the solenoid valves, so as to change the injection amount of the reaction reagents entering the buffer reservoir. Optionally, a control module is integrally provided in the present integrated gas path control plate, and the control module is electrically connected to the solenoid valves, so as to realize automatic control on the flow of the driving gas.

As an optional technical solution of the present reaction module, the surface of the cell reaction plate on the side thereof attached to the integrated gas path control plate is further provided with a waste reservoir, and the waste reservoir communicates with the reaction chamber. A waste gas extraction channel (or microchannel) is provided inside the integrated gas path control plate, and the waste gas extraction channel communicates with the waste liquid reservoir. The reaction chamber, the waste liquid reservoir, and the waste liquid gas extraction channel communicate with each other in sequence in a gas extraction direction. A waste after the reaction ends in the reaction chamber is drawn into the waste reservoir by means of gas extraction in the waste liquid gas extraction channel.

It should be noted that the waste liquid gas extraction channel provided in the present reaction module participates in two process steps, specifically:

First, during the cell reaction, after the reaction reagents are injected into the buffer reservoir from the reagent reservoirs, the waste liquid gas extraction channel is used to perform gas extraction. Since the waste liquid reservoir, the reaction chamber, and the buffer reservoir communicate with each other in sequence, the reaction reagents temporarily stored in the buffer reservoir is drawn into the reaction chamber under the action of the suction. It should be particularly noted that the negative pressure for gas extraction cannot be excessively large to prevent the reaction reagents entering the reaction chamber from being further drawn into the waste liquid reservoir.

Second, after the cell reaction ends, the waste liquid gas extraction channel is used again for performing gas extraction, and the reaction waste remaining in the reaction chamber is drawn into the waste reservoir under the action of the suction.

Optionally, a gas extraction end of the waste liquid gas extraction channel is provided with a solenoid valve, and the solenoid valve is used to control a gas extraction amount.

In the present reaction module, the solenoid valve connected to the waste liquid gas extraction channel is mainly used to control the gas extraction amount. As described above, the waste liquid gas extraction channel participates in two process steps. The two process steps both need to control the gas extraction amount. The gas extraction amount needs to be strictly controlled especially in the first process step to prevent excessively large negative pressure generated in gas extraction which causes the reaction reagents in the reaction chamber to be further drawn into the waste liquid reservoir. Thus, in the present reaction module, the gas extraction end of the waste liquid gas extraction channel is provided with the solenoid valve which, together with a control module, automatically controls the gas extraction amount.

As an optional technical solution of the present reaction module, the surface of the cell reaction plate on the side thereof attached to the integrated gas path control plate is further provided with a product reservoir, and the product reservoir communicates with the reaction chamber. A product gas extraction channel is provided inside the integrated gas path control plate, and the product gas extraction channel communicates with the product reservoir. The reaction chamber, the product reservoir, and the product gas extraction channel communicate with each other in sequence in a gas extraction direction, and a reaction product obtained in the reaction chamber is drawn into the product reservoir by means of gas extraction in the product gas extraction channel.

Optionally, a gas extraction end of the product gas extraction channel is provided with a solenoid valve, and the solenoid valve is used to control a gas extraction amount.

In the present reaction module, the solenoid valve connected to the product gas extraction channel is mainly used to control the gas extraction amount. After the cell reaction ends, the product gas extraction channel is used to perform gas extraction on the product reservoir, so that the reaction product in the reaction chamber enters the product reservoir. In order to draw the reaction product in the reaction chamber completely into the product reservoir, the gas extraction amount needs to be strictly controlled. Thus, in the present reaction module, the gas extraction end of the waste liquid gas extraction channel is provided with the solenoid valve which, together with the control module, automatically controls the gas extraction amount.

As an optional technical solution of the present reaction module, a silicone pad is sandwiched between the integrated gas path control plate and the cell reaction plate, a through hole is provided in the silicone pad, and the integrated gas path control plate and the cell reaction plate are connected by means of the through hole.

As an optional technical solution of the present reaction module, the solenoid valves are arranged in a concentrated manner on a surface of the integrated gas path control plate on a side thereof away from the cell reaction plate.

In the present reaction module, different types of solenoid valves are provided to achieve control of the entire flow path. Different solenoid valves implement different control functions. A conventional fluid solenoid valve has problems of occupying a large space and complex mounting and use. In the present cell reaction module, mounting positions are reserved for the solenoid valves on the surface of the integrated gas path control plate, so as to facilitate mounting and dismounting of the solenoid valves. In addition, the solenoid valves are integrally mounted on the surface of the integrated gas path control plate, so that a fluid can pass through the flow path inside the integrated gas path control plate to achieve control of the entire flow path.

Optionally, the integrated gas path control plate is further provided with an observation window, and a reaction condition within the reaction chamber is observed through the observation window.

The present disclosure also provides a method of using the cell reaction module as described herein to perform a cell reaction, the method comprising: injecting reaction reagents into the reagent reservoirs in advance, injecting a driving gas into the reagent reservoirs through the driving gas path channels, and making the reaction reagents in the reagent reservoirs flow into the reaction chamber under a pressure of the driving gas to perform the cell reaction.

As an optional technical solution of the present reaction module, the method specifically comprises:
(I) feeding a driving gas into the driving gas path channels through the solenoid valves, injecting the driving gas into corresponding reagent reservoirs along the independent driving gas path channels, and pressing the reaction reagents stored in the reagent reservoirs into the buffer reservoir under the pressure of the driving gas;
(II) performing gas extraction on the waste liquid reservoir through the waste liquid gas extraction channel, so that the reaction reagents in the buffer reservoir are drawn into the reaction chamber to complete reagent injection; and
(III) repeatedly performing step (I) and step (II), so that the reaction reagents in the reagent reservoirs are all injected into the reaction chamber to perform the cell reaction.

As an optional technical solution of the present method of performing a cell reaction using the cell reaction module described herein, the method further comprises: after the cell reaction ends, performing gas extraction on the waste liquid reservoir through the waste liquid gas extraction channel again, so that a waste liquid in the reaction chamber enters the waste liquid reservoir.

As an optional technical solution of the present method of preforming a cell reaction using the reaction module described herein, the method further comprises: after the cell reaction ends, performing gas extraction on the product reservoir through the product gas extraction channel, so that a reaction product in the reaction chamber enters the product reservoir.

As compared with the prior art, the beneficial effects of the present reaction module and method of use thereof can include one or more of the the following:

In the present reaction module, an integrated gas path control plate is provided with grooves therein to integrate various driving gas path channels. Some driving gas path channels are connected by means of solenoid valves, so as to realize connection or separation of different driving gas path channels according to needs through a control switch of an upper computer, and a driving gas is used as a driving force for injecting reaction reagents to transport the reaction reagents in a flow path. During use, the driving gas is injected into different driving gas path channels, so that reaction reagents in different reagent reservoirs can be injected into the reaction chamber one by one. The injection processes of different reaction reagents are independent of each other and do not affect each other, so that the flow path control process of the reaction reagents is stable and reliable, and the entire cell reaction module can be mounted easily and occupies a small space.

In a third specific embodiment, the present disclosure provides a sample preparation device and a preparation method using the device. Via combined use of a gas-path control board and a reaction chip and by using driving gas to control addition of a reaction reagent, the present sample preparation device can effectively control the amount of the reaction reagent added without causing contamination of the reaction reagent. Further, the present sample preparation device can include a heating plate, and is therefore capable of performing reverse transcription of a cell sample. The present sample preparation device has benefits such as a simple structure, a small footprint, convenient operation and high adaptability.

The present disclosure provides a sample preparation device. The preparation device includes a frame (also referred to as a housing herein), the frame is provided with a reaction module thereon. The reaction module includes a gas-path control board (also referred to as a gas-flow control device herein) and a heating plate, and a reaction chip (also referred to as a microfluidic device herein) is sandwiched between the gas-path control board and the heating plate. The gas-path control board is provided with at least two mutually independent driving gas-path channels therein. The reaction chip comprises a platform (also referred to as a reaction unit or a working unit herein) and a cell reaction plate (also referred to as a reagent exchange unit herein) attached to each other. A surface of the cell reaction plate on a side thereof attached to the gas-path control board is provided with reagent reservoirs of the same number as the driving gas-path channels. Each of the driving gas-path channels independently communicates with one reagent reservoir. A surface of the cell reaction plate on a side thereof attached to the platform is provided with a reaction chamber, and the reagent reservoirs are all independently connected to the reaction chamber. A reaction reagent is injected into the reagent reservoir in advance, and driving gas is injected into the reagent reservoir via the driving gas-path channel so as to press the reaction reagent in the reagent reservoir into the reaction chamber.

In the present sample preparation device, via the arrangement of the reagent reservoir on the reaction chip, in combination with injection of gas into the reagent reservoir via the driving gas-path channel on the gas-path control board, the injection of the reaction reagent into the reaction chamber to perform cell reaction is thereby achieved. The reaction reagent is injected into a buffer reservoir (also referred to as a reagent exchange reservoir herein) under the control of the driving gas, to achieve the injection of different reagents in batches or together, thereby achieving the quantitative injection of different reaction reagents, which effectively reduces the operation difficulty for an operator. The matching of structures of the gas-path control board and the reaction chip simplifies the structure of the reaction unit. Further, via the arrangement of the heating plate to heat the reaction chip, the present sample preparation device is capable of performing reverse transcription, and the present sample preparation device has benefits such as a simple structure, easy operation, a small footprint and high adaptability.

As an optional technical solution of the present sample preparation device, a surface of the cell reaction plate on the side thereof on which the reagent reservoir is located is further provided with a product reservoir and a waste reservoir. The gas-path control board is further provided with at least two gas extraction channels therein. The product reservoir and the waste reservoir are each independently connected to one of the two gas extraction channels.

Optionally, the product reservoir and the waste reservoir are independently connected to the reaction chamber.

Optionally, the product reservoir and the waste reservoir are each connected to a gas extraction solenoid valve via a gas extraction channel.

In the present sample preparation device, via the arrangement of the product reservoir and the waste liquid reservoir, which respectively communicate with the gas extraction solenoid valves, the cell sample in the reaction chamber can be drawn. In addition, by means of gas extraction performed on the waste reservoir, the reaction reagent in the buffer reservoir is driven into the reaction chamber. After the reaction, the cell sample from the reaction is collected to the product reservoir by subjecting the product reservoir to gas extraction.

As an optional technical solution of the present sample preparation device, the reagent reservoirs are all connected to a plurality of gas injection solenoid valves via the driving gas-path channels.

Optionally, the gas extraction solenoid valves and the plurality of gas injection solenoid valves are concentratedly arranged on a surface of the gas-path control board on the same side thereof.

In the present sample preparation device, the gas extraction solenoid valves and the plurality of gas injection solenoid valves are concentratedly arranged on a surface of the gas-path control board on the same side thereof, thereby improving the integration degree of the device, avoiding the problem of messy pipelines, and reducing a footprint.

As an optional technical solution of the present sample preparation device, a surface of the cell reaction plate on the side on which the reagent reservoir is located is provided with a buffer reservoir. The reagent reservoir and the reaction chamber independently communicate with the buffer reservoir. In a flow direction of the reaction reagents, the reagent reservoir, the buffer reservoir and the reaction chamber are connected in sequence.

Optionally, a silicone pad is arranged between the cell reaction plate and the gas-path control board, and the silicone pad is provided with a hole thereon corresponding to an outlet position of the driving gas-path channels on the gas-path control board.

As an optional technical solution of the present sample preparation device, the gas-path control board comprises an upper gas-path board and a lower gas-path board laminated to each other.

Optionally, at least one gas-path groove is arranged on an attaching surface between the lower gas-path board and the upper gas-path board; the upper gas-path board is attached to the lower gas-path board, so that the gas-path groove is closed and sealed to form the driving gas-path channels and the gas extraction channels.

As an optional technical solution of the present sample preparation device, the surface of the cell reaction plate on the side thereof attached to the platform is further provided with a plurality of independent reagent flow grooves, and after the platform is attached to the cell reaction plate and sealed, the reagent flow grooves form a plurality of reagent flow channels.

Optionally, the reagent reservoirs are independently connected to the buffer reservoir via the plurality of reagent flow channels. Optionally, the buffer reservoir is independently connected to the reaction chamber via a reagent flow channel. Optionally, both the product reservoir and the waste reservoir are independently connected to the reaction chamber via independent reagent flow channels.

As an optional technical solution of the present sample preparation device, the reaction module is provided with a control unit at the bottom thereof, and the control unit is independently electrically connected to the heating plate, the gas injection solenoid valve and the gas extraction solenoid valve, and independently controls activation of the heating plate, activation of the plurality of gas injection solenoid valves, and activation of the gas extraction solenoid valves.

As an optional technical solution of the present sample preparation device, the plurality of gas injection solenoid valves and the gas extraction solenoid valves are connected to a gas pump assembly configured to control gas pressure in the plurality of gas injection solenoid valves and the gas extraction solenoid valves.

Optionally, the gas pump assembly is located below the reaction module. Optionally, the gas pump assembly and the control unit are arranged side by side.

In the present sample preparation device, by integrally arranging the control unit and the gas pump assembly at the bottom of the reaction module, the footprint of the device is further reduced and the integration degree of the device is improved.

The present disclosure further provides a preparation method for preparing a cell sample by using the sample preparation device as described herein. The preparation method comprises: injecting a reaction reagent into the reagent reservoir, placing the reaction chip between the gas-path control board and the heating plate, injecting driving gas into the reagent reservoir via the driving gas-path channel, pressing the reaction reagent in the reagent reservoir into the reaction chamber to perform a cell reaction, and activating the heating plate to heat the reaction chamber to perform reverse transcription, to prepare the cell sample.

It needs to be noted that, the type of the reaction reagent is not particularly specified and specially restricted in the present sample preparation device, and those of skill in the art can reasonably select the type and the addition amount of the reaction reagent according to the type of the cell sample to be prepared.

As an optional technical solution of the present preparation method, the preparation method particularly comprises the following steps:
(I) after injecting reaction reagents into the reagent reservoirs, placing the reaction chip between the gas-path control board and the heating plate, activating, under the control of the control unit, a gas injection solenoid valve and pressing a reaction reagent into the buffer reservoir by injecting gas into the reagent reservoir connected to the gas injection solenoid valve, and activating, under the control of the control unit, the gas extraction solenoid valve connected to the waste reservoir, and extracting gas from the waste reservoir to draw the reaction reagent in the buffer reservoir into the reaction chamber to perform cell reaction;
(II) repeating the operation of Step (I) at least once, and controlling the heating plate using the control unit to heat the reaction chamber to perform the reverse transcription, and obtaining the cell sample; and
(III) activating, under the control of the control unit, the gas extraction solenoid valve connected to the product reservoir, and drawing the cell sample in the reaction chamber into the product reservoir.

Compared with the prior art, the present cell sample preparation device and method of use thereof can have some or all of the following beneficial effects:

Via the arrangement of the reagent reservoirs on the reaction chip, in combination with injection of gas into the reagent reservoirs via the driving gas-path channels on the gas-path control board, the injection of the reaction reagents into the reaction chamber to perform cell reaction is thereby achieved. The reaction reagents are injected into a buffer reservoir under the control of the driving gas, to achieve the injection of different reagents in batches or together, thereby achieving the quantitative injection of different reaction reagents, which effectively reduces the operation difficulty for an operator. The matching of structures of the gas-path control board and the reaction chip simplifies the structure of the reaction unit. Further, via the arrangement of the heating plate to heat the reaction chip, the present sample preparation device is capable of performing reverse transcription, and the present sample preparation device has benefits such as a simple structure, easy operation, a small footprint and high adaptability.

In a fourth specific embodiment, the present disclosure provides an integrated reaction system (also referred to herein as a sample preparation system or a reaction system) for preparing a single-cell sample and a method for carrying out a cell reaction using the same. The reaction system for preparing a single-cell sample in the present disclosure is provided with a drive module to achieve automatic operation for the whole process of cell preparation, thereby implementing automatic labelling of molecular tags, and achieving automation of RNA reverse transcription. A DNA product having a certain temperature is directly produced by the device, and the whole process from cell suspension to production of the DNA product is automated, thereby reducing an operation threshold for experimenter staff, and simplifying operation processes.

The present disclosure provides an integrated reaction system for preparing a single-cell sample. The reaction system comprises: an integrated gas-path control module, a cell reaction module and a drive module;
the integrated gas-path control module is located above the cell reaction module, and the drive module is divided into a horizontal movement module and a vertical movement module;
the cell reaction module comprises a reaction platform, and at least two cell reaction plates (also referred to as a microfluidic device herein) arranged side by side on the reaction platform; the integrated gas-path control module comprises a gas-path platform, and at least two integrated gas-path control boards (also referred to as a gas-flow control device herein) arranged side by side on the gas-path platform, and locations of the cell reaction plates correspond to those of the integrated gas-path control boards;
the reaction platform is fixed on the horizontal movement module, and the gas-path platform is fixed on the vertical movement module; the horizontal movement module drives the reaction platform to move away from directly below the integrated gas-path control module, the cell reaction plates into which the reaction reagents have been injected are fixed on the reaction platform, then the horizontal movement module drives the reaction platform to return to the original position, and the vertical movement module drives the gas-path platform to press downward such that the integrated gas-path control boards are attached to the cell reaction plates;
each integrated gas-path control board is internally provided with at least two mutually independent driving gas channels, reagent reservoirs are provided on a side surface of a cell reaction plate attached to the integrated gas-path control board, and the number of the reagent reservoirs is the same as the number of the driving gas channels, and each driving gas channel independently communicates with a reagent reservoir; a reaction chamber is provided on a side surface of the cell reaction plate located away from the integrated gas-path control board, and is in communication with the reagent reservoirs into which the reaction reagents are injected in advance, and a driving gas is injected into the reagent reservoirs via the driving gas channels, such that the reaction reagents in the reagent reservoirs are driven by the pressure of the driving gas to flow into the reaction chamber.

The existing high-throughput device for preparing a single-cell sample has a low degree of automation, and the automated procedure only covers automatic labelling of molecular tags. However, the present reaction system is provided with a drive module to achieve automatic operation for the whole process of cell preparation, thereby implementing automatic labelling of molecular tags, and achieving automation of RNA reverse transcription. A DNA product can be directly produced by the device at a certain temperature, and the whole process from cell suspension to production of the DNA product is automated, thereby reducing an operation threshold for experiment staff, and simplifying operation processes.

The present reaction system for preparing a single-cell sample is mainly composed of an integrated gas-path control module, a cell reaction module and a drive module. The device can prepare multiple groups of cell samples at the same time, so as to shorten the operation time, improve preparation efficiency, and further reduce the equipment size.

In the present device, recesses (or grooves) are provided inside the integrated gas-path control board, such that various driving gas channels are integrally formed. The various driving gas channels are independent of each other and are not in communication with each other. The driving gas is used as a driving force for injecting the reaction reagents, so as to convey the reaction reagents in flow paths. During use, the driving gas is injected into different driving gas channels, such that the reaction reagents in the different reagent reservoirs can be injected into the reaction chamber one by one. The injection processes of different reaction reagents are independent of each other and do not affect each other, so that flow path control processes of the reaction reagents are stable and reliable, and the cell reaction module as a whole can be easily installed and occupies a reduced space.

It should be noted that the formation manner of the driving gas channel (or microchannel) is not particularly specified or defined in the present reaction system, as long as smooth transportation of the driving gas is achieved. Exemplarily, the following two solutions, or a combination thereof, may be used:
Solution 1: the integrated gas-path control board is an integrated structure, and the driving gas channels are directly provided inside the integrated gas-path control board by means of casting, drilling, additive manufacturing, or the like; and
Solution 2, the integrated gas-path control board is a separable structure, and is formed by stacking and attaching an upper gas-path board and a lower gas-path board together. Gas-path recesses (or grooves) are provided at an attachment surface between the upper gas-path board and/or the lower gas-path broad, and after the upper gas-path board is attached to the lower gas-path broad, the gas-path recesses (or grooves) are sealed to form driving gas channels. Certainly, it can be understood that in this solution, the gas-path recesses (or grooves) can be arranged on the lower surface of the upper gas-path board or the upper surface of the lower gas-path board, or can be arranged on both the lower surface of the upper gas-path board and the upper surface of the lower gas-path board.

It should be noted that the cell reaction plate is similarly provided with fluid microchannels enabling reaction reagents to flow therein. For the formation manner of the fluid microchannels, reference is made to the above description of the driving gas channels. That is, the cell reaction plate can be an integrated structure or a separable structure. When the cell reaction plate is an integrated structure, the microchannels are directly provided inside the cell reaction plate by means of casting, drilling, additive manufacturing, or the like. Certainly, the cell reaction plate can also be a separable structure formed by stacking and attaching an upper reaction plate and a lower reaction plate together. Flow channel recesses (or grooves) are provided at an attachment surface between the upper reaction plate and/or the lower reaction plate, and after the upper reaction plate is attached to the lower reaction plate, the flow channel recesses (or grooves) are sealed to form the microchannels.

It should be noted that the number of the reagent reservoirs is not specifically defined in the present reaction system, the number of the reagent reservoirs is the same as that of the driving gas channels, and an outlet end of each driving gas channel corresponds to one reagent reservoir. Those skilled in the art can design the number and position of the reagent reservoirs specifically according to different cell reactions.

As an optional technical solution of the present reaction system, the device further comprises a control module (also referred to as a control unit herein) used to independently control the horizontal movement module and the vertical movement module.

Optionally, the reaction platform is internally provided with a heating element electrically connected to the control module, and the control module is configured to control a heating temperature of the heating element.

In the present device for preparing a single-cell sample, the heating element is integrated into the reaction platform, and a reaction temperature is accurately controlled by means of the control module. When the reaction system is operating at the stage of RNA reverse transcription, the heating element provides an accurate and controllable temperature range for the reaction.

As an optional technical solution of the present reaction system, the device further comprises a base (also referred to as a housing herein) configured to support and fix the integrated gas-path control module, the cell reaction module, the drive module, and the control module.

As an optional technical solution of the present reaction system, the horizontal movement module comprises sliding table assemblies arranged side by side on the base, and the sliding table assemblies are fixed on a bottom surface of the reaction platform, and are used to support the cell reaction module and to pull and move the cell reaction module in a horizontal direction.

Optionally, each of the sliding table assemblies comprises a sliding table, a sliding table support base and a stepping motor. The sliding table is fixed on the bottom surface of the reaction platform, and is mounted on the sliding table support base, one end of the sliding table is connected to an output shaft of the stepping motor, and the sliding table is driven by the stepping motor to move in the horizontal direction on the sliding table support base. Optionally, the vertical movement module comprises push-rod assemblies vertically fixed to two ends of a bottom surface of the gas-path platform. Each of the push-rod assemblies comprises a slide rail and a gear rack provided in the slide rail. One end of the gear rack is fixed to the edge of one end of the gas-path platform. Two parallel gear shafts are disposed on a surface of the base. A drive motor is provided at one end of each gear shaft, and the gear shaft is driven by the drive motor to rotate, so as to drive the gear rack to move in the vertical direction.

It should be noted that the movement logic of the integrated gas-path control module and the cell reaction module in the present reaction system is as follows: in an initial state, the cell reaction module is located directly below the gas-path control module; before starting of a cell reaction, the cell reaction module needs to be pulled in a horizontal direction, and the operator takes out the cell reaction plate, injects reaction reagents into respective reagent reservoirs on the cell reaction plate, and subsequently fixes the cell reaction plate on the reaction platform; the reaction platform together with the cell reaction plate supported thereon is moved and returned to the original position in the horizontal direction, so as to move to directly below the integrated gas-path control module again; at this moment, the integrated gas-path control module is pressed downward and attached to the cell reaction module, allowing outlet ports of the driving gas channels in the integrated gas-path control board to align with the corresponding reagent reservoirs on the cell reaction plate.

In the present reaction system, the horizontal movement module is configured to pull and move the reaction platform in a horizontal direction, and the vertical movement module is configured to drive the gas-path platform in a vertical direction. The specific structures and driving manners of the horizontal movement module and the vertical movement module are not particularly specified or limited.

As an optional technical solution of the present reaction system, a side surface of the cell reaction plate attached to the integrated gas-path control board is further provided with a buffer reservoir. The reagent reservoir and the reaction chamber independently communicate with the buffer reservoir. In a flow direction of the driving gas, the driving gas channel, the reagent reservoir, and the buffer reservoir communicate with each other in sequence. The reaction reagents stored in the respective reagent reservoirs are driven to the buffer reservoir one by one, and are injected into the reaction chamber via the buffer reservoir.

Optionally, a solenoid valve is provided at an inlet end of the driving gas channels and is electrically connected to the control module. An opening degree of the solenoid valve is controlled by the control module, so as to adjust an inlet amount of the driving gas.

In the present reaction system, the solenoid valve connected to the driving gas channels is configured to control a flow amount of the driving gas. During use of the cell reaction module provided in the present reaction system, the reaction reagent stored in the reagent reservoir is driven by the pressure of the driving gas into the buffer reservoir. The flow amount of the driving gas is adjusted by the solenoid valve, so as to control the injection amount of the reaction reagent into the buffer reservoir. Optionally, a control module is integrated with the integrated gas-path control board provided in the present reaction system, and is electrically connected to the solenoid valve, so as to achieve automatic control of the flow amount of the driving gas.

As an optional technical solution of the present reaction system, the side surface of the cell reaction plate attached to the integrated gas-path control board is further provided with a waste reservoir in communication with the reaction chamber. The integrated gas-path control board is internally provided with a waste gas extraction channel (or microchannel) in communication with the waste reservoir. In a gas extraction direction, the reaction chamber, the waste liquid reservoir, and the waste liquid gas extraction channel communicate with each other in sequence. Gas extraction is performed by means of the waste gas extraction channel, such that a waste obtained after completion of a reaction in the reaction chamber is drawn into the waste reservoir.

It should be noted that the waste liquid gas extraction channel provided in the present reaction system participates in two process steps, and specifically comprises:
firstly, during the cell reaction, after the reaction reagents are injected into the buffer reservoir from the reagent reservoirs, outward gas extraction is performed by means of the waste gas extraction channel. Since the waste reservoir, the reaction chamber and the buffer reservoir communicate with each other in sequence, the reaction reagents temporarily stored in the buffer reservoirs are drawn into the reaction chamber under the action of a suction force. At this moment, it should be particularly noted that in order to prevent reaction reagents entering the reaction chamber from being further drawn into the waste liquid reservoir, the negative pressure for gas extraction should not be excessively large; and
secondly, after the cell reaction is complete, gas extraction is performed again by means of the waste gas extraction channel, and the reaction waste remaining in the reaction chamber is drawn into the waste reservoir under the action of a suction force.

Optionally, a solenoid valve is provided at a gas extraction end of the waste liquid gas extraction channel and is electrically connected to the control module. An opening degree of the solenoid valve is controlled by the control module, so as to adjust a gas extraction amount.

In the present reaction system, the solenoid valve connected to the waste gas extraction channel is used for controlling the gas extraction amount. As disclosed herein the waste liquid gas extraction channel participates in two process steps. The two process steps both need to control the gas extraction amount. The gas extraction amount needs to be strictly controlled especially in the first process step to prevent excessively large negative pressure generated in gas extraction which causes the reaction reagents in the reaction chamber to be further drawn into the waste liquid reservoir. Thus, in the present reaction system, the gas extraction end of the waste liquid gas extraction channel is provided with the solenoid valve which, together with the control module, automatically controls the gas extraction amount.

As an optional technical solution of the present reaction system, the side surface of the cell reaction plate attached to the integrated gas-path control board is further provided with a product reservoir in communication with the reaction chamber. The integrated gas-path control board is internally provided with a product gas extraction channel (or microchannel) in communication with the product reservoir. In a gas extraction direction, the reaction chamber, the product reservoir, and the product gas extraction channel communicate with each other in sequence. Gas extraction is performed by means of the product gas extraction channel, such that a reaction product obtained in the reaction chamber is drawn into the product reservoir.

Optionally, a solenoid valve is provided at a gas extraction end of the product gas extraction channel and is electrically connected to the control module. An opening degree of the solenoid valve is controlled by the control module, so as to adjust a gas extraction amount.

In the present reaction device, the solenoid valve connected to the product gas extraction channel is configured to control the gas extraction amount. After the cell reaction is complete, gas is extracted from the product reservoir through the product gas extraction channel, such that a reaction product in the reaction chamber enters the product reservoir. In order to draw the reaction product in the reaction chamber completely into the product reservoir, the gas extraction amount needs to be strictly controlled. Therefore, in the present reaction system, the gas extraction end of the waste liquid gas extraction channel is provided with a solenoid valve which, together with the control module, automatically controls the gas extraction amount.

As an optional technical solution of the present reaction system, a silicone pad is sandwiched between the integrated gas-path control board and the cell reaction plate and is provided with a through-hole, and the integrated gas-path control board and the cell reaction plate communicate with each other via the through-hole.

Optionally, the solenoid valves are concentratedly arranged on a side surface of the integrated gas-path control board located away from the cell reaction plate.

The present reaction system achieves control of the entire flow path by arranging different types of solenoid valves. The different solenoid valves implement different control functions. Conventional fluid solenoid valves, during installation and use, have problems such as occupying a large space and requiring complex installation processes. In the present reaction system, installation locations for solenoid valves are reserved on the surface of the integrated gas-path control board, so as to facilitate installation and removal of the solenoid valves. In addition, the solenoid valves are integrated with the surface of the integrated gas-path control board, so that a fluid can pass through the flow path inside the integrated gas-path control board, thereby achieving control of the entire flow path.

Optionally, the integrated gas-path control board is further provided with an observation window used to observe a reaction situation in the reaction chamber.

The present disclosure also provides a method for carrying out a cell reaction using the cell reaction module described herein. The method comprises: injecting reaction reagents into reagent reservoirs in advance; driving, by a horizontal movement module, a reaction platform to move away from directly below an integrated gas-path control module; fixing, on the reaction platform, a cell reaction plate into which the reaction reagents have been injected; and then driving the reaction platform to return to the original position by means of the horizontal movement module; driving, by a vertical movement module, a gas-path platform to press downward such that an integrated gas-path control board is attached to a cell reaction plate; and injecting a driving gas into the reagent reservoirs via driving gas channels, such that the reaction reagents in the reagent reservoirs are driven by the pressure of the driving gas into a reaction chamber; and carrying out a cell reaction.

As an optional technical solution, the method comprises:
(I) injecting the reaction reagents into the reagent reservoirs in advance; driving, by the horizontal movement module, the reaction platform to move away from directly below the integrated gas-path control module; fixing, on the reaction platform, the cell reaction plate into which the reaction reagents have been injected; then driving the reaction platform to return to the original position by means of the horizontal movement module; and driving, by the vertical movement module, the gas-path platform to press downward such that the integrated gas-path control board is attached to the cell reaction plate;
(II) injecting a driving gas into the driving gas channels via a solenoid valve; injecting the driving gas into the reagent reservoirs along the independent driving gas channels corresponding thereto; driving the reaction reagents stored in the reagent reservoirs into a buffer reservoir by means of the pressure of the driving gas; and extracting gas from a waste reservoir through a waste gas extraction channel, such that the reaction reagents in the buffer reservoir are drawn into the reaction chamber to complete reagent injection, and performing a cell reaction; and
(III) after the cell reaction is complete, performing gas extraction with respect to the waste liquid reservoir through the waste liquid gas extraction channel again, such that a waste liquid in the reaction chamber enters the waste liquid reservoir; and extracting gas from a product reservoir through a product gas extraction channel, such that a reaction product in the reaction chamber enters the product reservoir.

Compared with the prior art, the present device for preparing a single-cell sample and method of use thereof can have one or more of the following beneficial effects:
(1) The existing high-throughput device for preparing a single-cell sample has a low degree of automation, and the automated procedure only covers automatic labelling of molecular tags. However, the present reaction system for preparing a single-cell sample is provided with a drive module to achieve automatic operation for the whole process of cell preparation, thereby implementing automatic labelling of molecular tags, and achieving automation of RNA reverse transcription. A DNA product having a certain temperature is directly produced by the device, and the whole process from cell suspension to production of the DNA product is automated, thereby reducing an operation threshold for experiment staff, and simplifying operation processes.
(2) The present reaction system for preparing a single-cell sample includes an integrated gas-path control module, a cell reaction module and a drive module. The reaction system can prepare multiple groups of cell samples at the same time, so as to shorten the operation time, improve preparation efficiency, further reduce the equipment size, and improve capture stability and accuracy.
(3) In the present reaction system, recesses (or grooves) are provided inside the integrated gas-path control board, such that various driving gas channels are integrally formed. The various driving gas channels are independent of each other and are not in communication with each other. The driving gas is used as a driving force for injecting the reaction reagents, so as to convey the reaction reagents in flow paths. During use, the driving gas is injected into different driving gas channels, such that the reaction reagents in the different reagent reservoirs can be injected into the reaction chamber one by one. The injection processes of different reaction reagents are independent of each other and do not affect each other, so that flow path control processes of the reaction reagents are stable and reliable, and the cell reaction module as a whole can be easily installed and occupies a reduced space.

Disclosed herein include microfluidic devices (or microfluidic chips). The microfluidic devices can be used for analysis, such as cell analysis, including single cell analysis. In some embodiments, a microfluidic device comprises a reagent (or solution) exchange unit (or exchange unit plate or module). Reagent exchange (or solution exchange) can occur at the reagent exchange unit. The reagent exchange unit can be a top unit (relative to the reaction unit). The reagent exchange unit can comprise a plurality of reagent (or solution) reservoirs (or containers or holders) on a surface (e.g., an upper surface) of the reagent exchange unit. The reagent exchange unit can comprise a reagent exchange reservoir on a surface (e.g., an upper surface) of the reagent exchange unit. The plurality of reagent reservoirs and the reagent exchange unit can be on the same surface (e.g., an upper surface) of the reagent exchange unit. The microfluidic device can comprise a reaction unit. A reaction can occur in the reaction unit. The reaction unit can be a bottom unit (relative to the reagent exchange unit). The reaction unit can be where reactions occur. The reaction unit can comprise a microarray and be referred to as a microarray unit. The microfluidic device can comprise a reaction chamber (e.g., where a reaction can occur) formed between a lower surface of the reagent exchange unit and a upper surface of reaction unit. The microfluidic device can comprise a plurality of fluid microchannels formed between a lower surface of the reagent exchange unit and a upper surface of reaction unit. The plurality of fluid microchannels can comprise a plurality of reagent fluid microchannels (or input fluid microchannels). Reagents can flow between reservoirs, such as between reagent reservoirs and the reagent exchange reservoir, via the plurality of reagent fluid microchannels. Fluid microchannels can be separate microchannels such that no two fluid microchannels are directly connected. Fluid microchannels can also be referred to herein as fluid channels. The reaction chamber and the plurality of fluid microchannels can be formed between a lower surface of the reagent exchange unit and a upper surface of reaction unit. The reaction chamber can comprise an inlet. The inlet can be connected to the reagent exchange reservoir, for example, directly or indirectly through a fluid microchannel. The reaction chamber can comprise an outlet. The outlet can be connected to a waste reservoir or a product reservoir, for example. Fluid microchannels (two or more, such as three or more, including all fluid microchannels) of the plurality of fluid microchannels can connect (i) reagent reservoirs (two or more, such as three or more, including all reagent reservoirs) of the plurality of reagent reservoirs and (ii) the reagent exchange reservoir. Different fluid microchannels can connect (i) different reagent reservoirs and (i) the reagent exchange reservoir. A different fluid microchannel can connect (i) a different reagent reservoir and (ii) the reagent exchange reservoir. The reagent exchange reservoir can be connected with the inlet of the reaction chamber.

In some embodiments, a microfluidic device comprises a reagent exchange unit. A reagent exchange unit can comprise a plurality of reagent reservoirs. The reagent exchange unit can comprise at least one reagent exchange reservoir. The reagent exchange unit can comprise a reaction unit. The reagent exchange unit can comprise a reaction chamber and a plurality of fluid microchannels formed between a surface (e.g., a lower surface) of the reagent exchange unit and a surface of reaction unit (e.g., an upper surface). One, one or more, or each of fluid microchannels of the plurality of fluid microchannels can connect (i) a reagent reservoir of the plurality of reagent reservoirs and (ii) the reagent exchange reservoir. The reagent exchange reservoir can be connected with an inlet of the reaction chamber.

In some embodiments, a microfluidic device comprises a plurality of reagent reservoirs. The microfluidic device can comprise at least one reagent exchange reservoir. The microfluidic device can comprise a reaction chamber. The microfluidic device can comprise a plurality of fluid microchannels. One, one or more, or each of fluid microchannels of the plurality of fluid microchannels can connect (i) a reagent reservoir of the plurality of reagent reservoirs and (ii) the reagent exchange reservoir. The reagent exchange reservoir can be connected with an inlet of the reaction chamber.

In some embodiments, a microfluidic device comprises a plurality of reagent reservoirs. The microfluidic device can comprise at least one reagent exchange reservoir. The microfluidic device can comprise a reaction chamber. The microfluidic device can comprise a plurality of fluid microchannels. Different fluid microchannels of the plurality of fluid microchannels can connect (i) different reagent reservoirs of the plurality of reagent reservoirs and (ii) the reagent exchange reservoir. The reagent exchange reservoir can be connected with (e.g., in fluid communication with) the reaction chamber.

In some embodiments, a microfluidic device comprises a plurality of reservoirs. The plurality of reservoirs can comprise, for example, one or more input reservoirs, such as reagent reservoirs and a reagent exchange reservoir. The plurality of reservoirs can comprise, for example, one or more output reservoirs, such as a waste reservoir and a product reservoir. The microfluidic device can comprise a reaction chamber. The microfluidic device can comprise a plurality of fluid microchannels. Each of the plurality of reservoirs can be connected with at least one other reservoir of the plurality of reservoirs via a fluid microchannel of the plurality of fluid microchannels. The plurality of fluid microchannels can comprise, for example, reagent fluid microchannels (or input fluid microchannels), such as fluid microchannels that connect reagent reservoirs with the reagent exchange reservoir. The reagent exchange reservoir can be connected directed to the inlet of the reaction chamber or indirectly via a fluid microchannel. The plurality of fluid microchannels can comprise, for example, output fluid microchannels, such as fluid microchannels that connect the reaction chamber with the waste reservoir and the product reservoir. At least one reservoir (e.g., a reagent exchange reservoir) of the plurality of reservoirs can be connected with at least two other reservoirs of the plurality of reservoirs. The at least one reservoir can be in fluid communication with the reaction chamber. One or more other reservoirs (e.g., waste reservoir, and product reservoir) can be in fluid communication with the reaction chamber.

In some embodiments, a microfluidic device comprises: a plurality of reservoirs. The microfluidic device can comprise a reaction chamber. The microfluidic device can comprise a plurality of fluid microchannels. One, one or more, or each of the plurality of reservoirs can be connected with (i) at least one other reservoir of the plurality of reservoirs via a fluid microchannel of the plurality of fluid microchannels, (ii) the reaction chamber directly, and/or (iii) the reaction chamber via a fluid microchannel of the plurality of fluid microchannels. For example, reagent reservoirs and a reagent exchange reservoir can each be connected with at least one other reservoir via fluid microchannels. For example, the reagent exchange reservoir can be connected to the reaction chamber directly or via a microfluidic channel. For example, a waste reservoir and a product reservoir can be connected to the reaction chamber. At least one reservoir (e.g., a reagent exchange reservoir) of the plurality of reservoirs is connected with at least two other reservoirs (e.g., reagent reservoirs) of the plurality of reservoirs.

In some embodiments, the microfluidic device comprises a first layer (e.g., a top layer) and a second layer (e.g., a bottom layer) reversibly coupled (e.g., bonded) to each other. The first layer can comprise a plurality of grooves. The second layer can cover the plurality of grooves. The plurality of grooves covered by the second layer can form the plurality of fluid microchannels. The first layer can comprise a cavity. The second layer can cover the cavity. The cavity covered by the second layer can form the reaction chamber.

In some embodiments, a microfluidic device comprises a reagent exchange unit and a reaction unit bonded (e.g., attached) to each other (e.g., reversibly or irreversibly). A first surface (e.g., an upper surface) of the reagent exchange unit can comprise a plurality of reagent reservoirs, a product reservoir, a waste reservoir, and/or a reagent exchange reservoir. All reagent reservoirs of the plurality of reagent reservoirs, the product reservoir, and/or the waste reservoir can be connected to the reagent exchange reservoir through a plurality of fluid microchannels and/or a reaction chamber on a second surface (e.g., a bottom surface) of the reagent exchange unit. The reaction unit (e.g., an upper surface) can cover the plurality of microchannels and the reaction chamber. The reaction unit can form, together with the second surface of the reagent exchange unit, the plurality of microchannels and the reaction chamber of the microfluidic device.

In some embodiments, a microfluidic device comprises a reagent exchange unit and a reaction unit bonded (e.g., attached) to each other (e.g., reversibly or irreversibly). An upper surface of the reagent exchange unit can comprise a plurality of reagent reservoirs, a product reservoir, a waste reservoir, and/or a reagent exchange reservoir. All reagent reservoirs of the plurality of reagent reservoirs, the product reservoir, and/or the waste reservoir can be connected to the reagent exchange reservoir through a plurality of fluid microchannels and/or a reaction chamber on a lower surface of the reagent exchange unit and in a recess of the lower surface of the reagent exchange unit. The recess can be connected to the reagent exchange reservoir, the product reservoir, and/or the waste reservoir. The reaction unit can cover the plurality of microchannels, the reaction chamber, and/or the recess, and forms, together with the recess and the lower surface of the reagent exchange unit, the plurality of microchannels and the reaction chamber of the microfluidic device.

In some embodiments, the reagent exchange unit is in direct contact with the reaction unit. The reagent exchange unit and the reaction unit can be bonded (e.g., attached) to each other (e.g., reversibly or irreversibly). The reagent exchange unit and the reaction unit can form an integral structure.

In some embodiments, the reagent exchange unit further comprises a waste reservoir on the upper surface (or top surface) of the reagent exchange unit. A waste fluid microchannel of the plurality of fluid microchannels can connect the waste reservoir and the outlet of the reaction chamber (directly, or indirectly through a fluid microchannel). The waste fluid microchannel can directly connect the waste reservoir and the outlet of the reaction chamber.

In some embodiments, the reagent exchange unit further comprises a product reservoir on the upper surface of the reagent exchange unit. A product fluid microchannel of the plurality of fluid microchannels can connect the product reservoir and the outlet of the reaction chamber. The product fluid microchannel can directly connect the product reservoir and the outlet of the reaction chamber.

In some embodiments, the waste fluid microchannel, the product fluid microchannel, and the outlet of the reaction chamber connect at a junction (e.g., a Y junction or a T junction). The waste fluid microchannel and the product fluid microchannel can merge into a single fluid microchannel which is then connected to the outlet of the reaction chamber.

In some embodiments, the plurality of reagent reservoirs comprises a mixing reservoir. A mixing fluid microchannel of the plurality of fluid microchannels can connect the mixing reservoir and the reagent exchange reservoir. The mixing fluid microchannel can split into two or more fluid microchannels which merge into a single fluid microchannel between the reagent exchange reservoir and the mixing reservoir. Alternatively or additionally, a first portion of the mixing fluid microchannel connects the mixing reservoir and a mixing chamber. A second portion of the mixing fluid microchannel can connect the mixing chamber and the reagent exchange reservoir. Between the first portion of the mixing fluid microchannel and the first portion of the mixing fluid microchannel, the mixing fluid microchannel splits into two fluid microchannels.

In some embodiments, one or more reagent reservoirs of the plurality of reagent reservoirs, the waste reservoir, and/or the product reservoir each comprises an opening (e.g., a hole) that connects the reservoir to a fluid microchannel of the plurality of fluid microchannels. The reagent exchange reservoir can comprise one or more openings (e.g., holes) that connect the reagent exchange reservoir to one or more fluid microchannels of the plurality of fluid microchannels. The reagent exchange reservoir can comprise an opening (e.g., a hole) that connects the reagent exchange reservoir to the inlet of the reaction chamber.

In some embodiments, one or more reagent reservoirs of the plurality of reagent reservoirs, the reagent exchange reservoir, the waste reservoir, and/or the product reservoir each is formed by a wall protruding from the upper surface of the reagent exchange unit. One or more reagent reservoirs of the plurality of reagent reservoirs, the reagent exchange reservoir, the waste reservoir, and/or the product reservoir each is formed by a wall protruding from the upper surface of the reagent exchange unit each can comprise a tapered bottom surface and/or a rounded bottom surface. The tapered bottom surface, or a portion thereof, and/or the rounded bottom surface, or a portion thereof, can be disposed in or can protrude into the upper surface of the reagent exchange unit.

In some embodiments, the plurality of reagent reservoirs can comprise at least two reagent reservoirs. Fluid microchannels of the plurality of fluid microchannels connecting reagent reservoirs of the plurality of reagent reservoirs to the reagent exchange reservoirs can comprise at least two fluid microchannels. The number of reagent reservoirs and the number of the fluidic microchannels connecting the reagent reservoirs to the reagent exchange reservoir can be identical.

In some embodiments, the upper surface of the reagent exchange unit is divided into a first area and a second area (e.g., a first functional area and a second functional area). The first area can comprise the product reservoir and the waste reservoir. The second functional area can comprise at least two reagent reservoirs. The second functional area can comprise the reagent exchange reservoir.

In some embodiments, one, one or more, or each of the plurality of reagent reservoirs comprises a reagent. Two of the plurality of reagent reservoirs can comprises different reagent, Each of the plurality of reagent reservoirs can comprise different reagents. Two of the plurality of reagent reservoirs can comprise an identical reagent.

In some embodiments, a cross-sectional shape of the product reservoir and a cross-sectional shape of the waste reservoir are identical. In some embodiments, a cross-sectional shape of the product reservoir and a cross-sectional shape of the waste reservoir are different. In some embodiments, a cross-sectional shape of a reservoir is a rectangle, a circle, an ellipse, a semicircle, a trapezoid, or a combination thereof. A cross-sectional shape of the product reservoir can be a rectangle, a circle, an ellipse, a semicircle, a trapezoid, or a combination thereof. A cross-sectional shape of the waste reservoir can be a rectangle, a circle, an ellipse, a semicircle, a trapezoid, or a combination thereof. A cross-sectional shape of one, one or more, of each of the plurality of reagent reservoirs can be a circle, a rectangle, an ellipse, a semicircle, a trapezoid, or a combination thereof. A cross-sectional shape of the reagent exchange reservoir can a circle, a rectangle, an ellipse, a semicircle, a trapezoid, or a combination thereof. A cross-sectional shape of the reaction chamber can be a circle, a rectangle, an ellipse, a semicircle, a trapezoid, or a combination thereof.

In some embodiments, a size (e.g., width, length, depth, radius, diameter, or circumference) of the product reservoir is 1 mm to 20 cm. A size (e.g., width, length, depth, radius, diameter, or circumference) of the waste reservoir can be 1 mm to 20 cm. A size (e.g., width, length, depth, radius, diameter, or circumference) of one, one or more, of each of the plurality of reagent reservoirs can be 1 mm to 20 cm. A size (e.g., width, length, depth, radius, diameter, or circumference) of the reagent exchange reservoir can be 1 mm to 20 cm. A size (e.g., width, length, depth, radius, diameter, or circumference) of the reaction chamber can be 1 mm to 20 cm. A size (e.g., width, length, depth, radius, diameter, or circumference) of the microfluidic device can be 1 mm to 20 cm. A size (e.g., width, length, depth, radius, diameter, or circumference) of the reagent exchange unit can be 1 mm to 20 cm. A size (e.g., width, length, depth, radius, diameter, or circumference) of the reaction unit can be 1 mm to 20 cm.

In some embodiments, a cross-sectional shape of a fluid microchannels is a circle, a rectangle, an ellipse, a semicircle, a trapezoid, or a combination thereof. A size (e.g., width, length, depth, radius, diameter, or circumference) of a fluid microchannel can be 1 mm to 20 cm.

In some embodiments, a shape of the microfluidic device is a circle, a rectangle, an ellipse, a semicircle, a trapezoid, or a combination thereof. A shape of the reagent exchange unit can be a circle, a rectangle, an ellipse, a semicircle, a trapezoid, or a combination thereof. A shape of the reaction unit can be a circle, a rectangle, an ellipse, a semicircle, a trapezoid, or a combination thereof. A size of the microfluidic device can be 1 cm to 30 cm. A size of the reagent exchange unit can be 1 cm to 30 cm. A size of the reaction can be 1 cm to 30 cm.

In some embodiments, the reaction chamber comprises two tapered ends forming the inlet and the outlet of the reaction chamber. In some embodiments, the reaction chamber comprises a microwell array comprising at least 100 microwells. The microwell array can be disposed on the upper surface of the reaction unit. In some embodiments, the lower surface of the reaction unit is capable of being (or is configured to be) in thermal contact with a heating element.

In some embodiments, the plurality of microchannels (or a plurality of grooves that forms the plurality of microchannels with the reaction unit) is in a recess of the lower surface of the reagent exchange unit. The reaction chamber (or a cavity that forms the reaction chamber with the reaction unit) can be in the recess of the lower surface of the reagent exchange unit. The reaction unit can cover the plurality of microchannels (or the plurality of grooves), the reaction chamber (or the cavity), and the recess. The reaction unit can form, together with the recess and/or the lower surface of the reagent exchange unit, the plurality of microchannels and the reaction chamber of the microfluidic device.

In some embodiments, the reagent exchange unit and/or the reaction unit comprises (i) the reaction chamber, or a portion thereof. The reagent exchange unit can comprise a cavity that is part of the reaction chamber. Alternatively or additionally, the reaction unit can comprise a cavity that is part of the reaction chamber. The reagent exchange unit and/or the reaction unit can comprise (ii) the plurality of fluid microchannels, or a portion of a fluid microchannel (or a portion of each of one or more fluid microchannels) of the plurality of fluid microchannels. The reagent exchange unit can comprise a groove that is part of a fluid microchannel. Alternatively or additionally, the reaction unit can comprise a groove that is part of a fluid microchannel.

In some embodiments, the lower surface of the reagent exchange unit and/or the upper surface of the reaction unit comprises (i) the reaction chamber, or a portion thereof. The lower surface of the reagent exchange unit and/or the upper surface of the reaction unit can comprise (ii) the plurality of fluid microchannels, or a portion of a fluid microchannel (or each of one or more fluid microchannels) of the plurality of fluid microchannels.

Disclosed herein include gas-flow control devices. A gas-flow control device can be used, for example with a microfluidic device for analysis, such as cell analysis, including single cell analysis. In some embodiments, a gas-flow control device comprises a plate (or a board, a platform, or scaffold). The gas-flow control device can comprise a plurality of gas injection valves disposed on and/or in (or through) the plate. The gas-flow control device can comprise a plurality of gas injection microchannels (or gas injection channels) disposed in the plate. Each gas injection microchannel can have an outlet open end on a lower surface (or bottom surface) of the plate. Each gas injection microchannel can be connected with one injection valve of the plurality of injection valves. A gas injection valve and a gas injection microchannel can be connected. The gas injection valve and the gas injection microchannel can be used to generate a positive pressure in a reagent reservoir for a reagent to flow from the reagent reservoir into the reagent exchange reservoir through a reagent fluid microchannel (or a input fluid microchannel).

In some embodiments, the plurality of gas injection valves comprises a plurality of reagent gas injection valves. The plurality of gas injection microchannels can comprise a plurality of reagent gas injection microchannels. A reagent gas injection valve and a corresponding reagent gas injection microchannel can be used to generate a positive pressure in a reagent reservoir, which can result in a reagent to flow from the reagent reservoir into reagent exchange reservoir, In some embodiments, the gas-flow control device can further comprises: a plurality of gas extraction valves disposed on and/or in (or through) the plate. The gas-flow control device can further comprise a plurality of gas extraction microchannels (or gas extraction channels) disposed in the plate. Each gas extraction microchannel can have an inlet open end on the lower surface of the plate. Each gas extraction microchannel can be connected with a gas extraction vale of the plurality of gas extraction valves. In some embodiments, the plurality of gas extraction valves comprises a product gas extraction valve and/or a waste gas extraction valve. The plurality of gas extraction microchannels can comprise a product gas extraction microchannel and/or a waste gas extraction microchannel. The product gas extraction microchannel can be connected with the product gas extraction valve. The product gas extraction valve and the product gas extraction microchannel can be used to generate a negative pressure in the product reservoir, which can result in one or more reagents in the reagent exchange reservoir to flow from the reagent exchange reservoir into the reaction chamber, then into the product reservoir. The waste gas extraction microchannel can be connected with the waste gas extraction valve. The waste gas extraction valve and the waste gas extraction microchannel can be used to generate a negative pressure in the waste reservoir, which can result in one or more reagents to flow from the reagent exchange reservoir into the reaction chamber, then into the waste reservoir.

In some embodiments, the plurality of gas extraction valves comprises a reagent exchange gas extraction valve. The plurality of gas extraction microchannels can comprise a corresponding reagent exchange gas extraction microchannel. The reagent exchange gas extraction vale and the gas extraction microchannel can be connected. The reagent exchange gas extraction vale and the gas extraction microchannel can be used to generate a negative pressure in the reagent exchange reservoir for a reagent to flow from a reagent reservoir into the reagent exchange reservoir through a reagent fluid microchannel. The plurality of gas injection valves comprises a reagent exchange gas injection valve. The plurality of gas injection microchannels can comprise a corresponding reagent exchange gas injection microchannel. The reagent exchange gas injection vale and the gas injection microchannel can be connected. The reagent exchange gas injection vale and the gas injection microchannel can be used to generate a positive pressure in the reagent exchange reservoir for a reagent to flow from the reagent exchange reservoir into the reaction chamber (or to a reagent reservoir, such as a mixing reagent reservoir, through a fluid microchannel). In some embodiments, the gas-flow control device comprises no gas extraction valve and no gas extraction microchannel for generating a negative pressure in the reagent exchange reservoir. The gas-flow control device can comprise no gas injection valve and no gas injection microchannel for generating a positive pressure in the reagent exchange reservoir.

In some embodiments, the plurality of gas extraction valves comprises a mixing gas extraction valve. The plurality of gas extraction microchannels can comprise a mixing gas extraction microchannel. The mixing gas extraction vale and the mixing gas microchannel can be connected. The mixing gas extraction vale and the mixing gas microchannel can be used to generate a negative pressure in the mixing reservoir, which can result in one or more reagents to flow from the reagent exchange reservoir into the mixing reservoir through the mixing fluid microchannel. The plurality of gas injection valves can comprise a mixing gas injection valve. The plurality of gas injection microchannels can comprise a mixing gas injection microchannel. The mixing gas injection valve and the mixing gas injection microchannel can be connected. The mixing gas injection valve and the mixing gas injection microchannel can be used to generate a positive pressure in the mixing reservoir, which can result in mixed reagents in the mixing reservoir to flow from the mixing reservoir into the reagent exchange reservoir.

In some embodiments, a gas-flow control device comprises a plurality of gas injection valves disposed on and/or in (or through) a plate of the gas-flow device. The gas-flow control device can comprise a plurality of gas extraction valves disposed on and/or in (or through) the plate of the gas-flow device. The gas-flow control device can comprise a plurality of gas injection microchannels disposed in the plate. Each gas injection microchannel can have an outlet open end on a lower surface of the plate. Each gas injection microchannel can have an inlet open end. The inlet open end can be connected with one injection valve of the plurality of injection valves. The gas-flow control device can comprise a plurality of gas extraction microchannels disposed in the plate. Each gas extraction microchannel can have an inlet open end on the lower surface of the plate. An outlet open end of a waste gas extraction microchannel can be connected to a waste gas extraction valve of the plurality of gas extraction valves. An outlet open end of a product gas extraction microchannel can be connected to a product gas extraction valve of the plurality of gas extraction valves.

In some embodiments, a gas-flow control device comprises a plurality of gas injection valves on and/or in (or through) a plate of the gas-flow device. The gas-flow control device can comprise and a plurality of gas extraction valves disposed on and/or in (or through) the plate of the gas-flow device. The gas-flow control device can comprise a plurality of gas injection microchannels disposed in the plate. Each gas injection microchannel can have an outlet open end on a lower surface of the plate. The gas injection microchannel can have an inlet open end connected with one injection valve of the plurality of injection valves. The gas-flow control device can comprise a plurality of gas extraction microchannels disposed in the plate. Each gas extraction microchannel can have an inlet open end on the lower surface of the plate. The gas extraction microchannel can have an outlet open end connected to a gas extraction valve of the plurality of gas extraction valves.

In some embodiments, a gas-flow control device comprises a plurality of gas injection microchannels disposed in a plate of the gas-flow control device. Each gas injection microchannel can have an outlet open end on a lower surface of the plate. The gas injection microchannel can have an inlet open end for connecting to one injection valve of a plurality of injection valves. The gas-flow control device can comprise a plurality of gas extraction microchannels disposed in the plate. Each gas extraction microchannel can have an inlet open end on the lower surface of the plate. The gas extraction microchannel can have an outlet open end for connecting to a gas extraction valve of a plurality of gas extraction valves.

In some embodiments, a gas-flow control device comprises a plurality of gas injection microchannels disposed in a plate of the gas-flow control device. Each gas injection microchannel can have an outlet open end on a lower surface of the plate and an inlet open end disposed within the plate. The gas-flow control device can comprise a plurality of gas extraction microchannels disposed in the plate. Each gas extraction microchannel can have an inlet open end on the lower surface of the plate. The gas extraction microchannel can have an outlet open end for connecting to a gas extraction valve disposed within the plate.

In some embodiments, the gas-flow control device further comprises a plurality of gas injection valves disposed on and in the plate, and the gas-flow control device can comprise a plurality of gas extraction valves disposed on and in the plate. The inlet open end of each of the plurality of gas injection microchannels can be connected to a gas injection valve of the plurality of gas injection valves. The outlet open end of each of the plurality of gas extraction microchannels can be connected to a gas extraction valve of the plurality of gas extraction valves.

In some embodiments, a majority of, or all of, the plurality of gas injection valves and/or a majority of, or all of, the plurality of gas extraction valves are arranged on an area (e.g., a function area) of the plate. The area can be on one end of the plate.

In some embodiments, one or more of the plurality of injection valves is not connected to a gas injection microchannel of the plurality of gas injection microchannels. One or more of the plurality of extraction valves is not connected to a gas extraction microchannel of the plurality of gas extraction microchannels.

In some embodiments, one, one or more, or each of the plurality of gas injection valves when pressurized with a driving gas and in an open state injects the driving gas in a direction (i) from the inlet open end of a gas injection microchannel, of the plurality of gas injection microchannels, connected to the gas injection valve (ii) to the outlet open end of the gas injection microchannel. One, one or more, or each of the plurality of gas extraction valves when under a suction and in an open state allows a gas (e.g., a gas in a reservoir, such as the waste reservoir or the product reservoir) to flow in a direction (i) from the inlet open end of a gas extraction microchannel, of the plurality of gas injection microchannels, connected to the gas extraction valve, (ii) to the outlet open end of the gas extraction microchannel.

In some embodiments, a gas injection valve of the plurality of gas injection valves controls an amount of gas entering the inlet open end and exiting the outlet open end of the corresponding gas injection microchannel. A gas extraction valve of the plurality of gas extraction valves can control an amount of gas entering the inlet open end and exiting the outlet open end of the corresponding gas extraction microchannel.

In some embodiments, one, one or more, or each of the plurality of gas injection valves is a solenoid valve. One, one or more, or each of the plurality of gas extraction valves can be a solenoid valve.

In some embodiments, the plate further comprises an observation window (e.g., an opening or a transparent section).

In some embodiments, a size of one, one or more, or each of the plurality gas injection microchannels is 1 mm to 20 cm. A size of the inlet and/or the outlet of one, one or more, or each of the plurality gas injection microchannels can be 0.1 mm to 5 mm. A size of one, one or more, or each of the plurality gas extraction microchannels can be 1 mm to 20 cm. A size of the inlet and/or the outlet of one, one or more, or each of the plurality gas extraction microchannels can be 0.1 mm to 5 mm. A size of the gas-flow control device can be 5 mm to 40 cm.

In some embodiments, a cross-sectional shape of one, one or more, or each of the plurality of gas injection microchannels is a circle, a rectangle, an ellipse, a semicircle, a trapezoid, or a combination thereof. A cross-sectional shape of one, one or more, or each of the plurality of gas extraction microchannels can be a circle, a rectangle, an ellipse, a semicircle, a trapezoid, or a combination thereof.

In some embodiments, the plate comprises a plurality of layers (or boards). Each of the plurality of layers can be coupled (or bonded), whether reversibly or irreversibly coupled), to at least one other layer of the plurality of layers. One or more gas injection valves of the plurality of gas injection valves can be disposed on and through a first layer (e.g., an upper layer) of the plurality of layers. One or more gas extraction valves of the plurality gas extraction valves can be disposed on and through the first layer. The first layer can comprise a plurality of grooves (e.g., a plurality of gas injection grooves and/or a plurality of gas extraction grooves). A second layer (e.g., a lower layer) of the plurality of layers can cover the plurality of grooves to form the plurality of gas injection microchannels and/or the plurality of gas extraction microchannels. In some embodiments, the one or more gas injection valves are disposed in a second layer of the plurality of layers. The one or more gas extraction valves can be disposed in the second layer of the plurality of layers. The second layer can be a cover layer. In some embodiments, the one or more gas injection valves are disposed through a second layer of the plurality of layers. The one or more gas extraction valves can be disposed through the second layer of the plurality of layers. The plurality of layers comprises a third layer that is a cover layer. The third layer can have a flat surface in contact with the second layer. Alternatively or additionally, the third layer can have a plurality of cavities into which the plurality of gas injection valves and the plurality of gas extraction valves are inserted. In some embodiments, one or more gas injection microchannels of the plurality of gas injection microchannels can be formed between and/or by the first layer and the second layer. One or more gas extraction microchannels of the plurality of gas extraction microchannels can be formed between and/or by the first layer and the second layer.

Disclosed herein include reaction modules (e.g., for analysis, such as cell analysis, including single cell analysis). In some embodiments, a reaction module comprises: a microfluidic device of the present disclosure. The reaction module can comprise a gas-flow control device of the present disclosure. The gas-flow control device can be capable of detachably coupling (e.g., attaching, or bonding) to the microfluidic device. The gas-flow control device can be capable of forming a seal, such as an air-tight seal, with the microfluidic device (or a portion thereof, such as one or more reservoirs of the microfluidic device). For example, the gas-flow control device can be capable of forming a seal, such as an air-tight seal, with some and not all reservoirs of the reagent exchange device (e.g., the reagent reservoirs, the mixing reservoir, the waste reservoir, and/or the product reservoir, and not the reagent exchange reservoir. For example, the gas-flow control device can be capable of forming a seal, such as an air-tight seal, with all reservoirs of the reagent exchange device.

In some embodiments, a reaction module comprises a microfluidic device described herein. The reaction module can comprise a gas-flow control device described herein. An area on a surface (e.g., a lower or bottom surface) of the gas-flow control device surrounding the outlet open end of one (or one or more, or each) gas injection microchannel of plurality of gas injection microchannels can be capable of detachably coupling to and/or forming a seal, such as an air-tight seal, with one reagent reservoir (or a corresponding reagent reservoir) of the plurality of reagent reservoirs. An area on the surface of the gas-flow control device surrounding the inlet open end of the waste gas extraction microchannel is capable of detachably coupling to and/or forming a seal, such as an air-tight seal, with the waste reservoir to result. An area on the surface of the gas-flow control device surrounding the inlet open end of the product gas extraction microchannel can be capable of detachably coupling to and/or forming a seal, such as an air-tight seal, with the product reservoir. In some embodiments, an outlet of a gas injection valve (or an inlet of a gas extraction valve) is open into the area not through a gas injection microchannel (or a gas extraction microchannel).

In some embodiments, a reaction module comprises a microfluidic device described herein. The reaction module can comprise a gas-flow control device described herein. The gas-flow control device can be capable of detachably coupling to and/or forming a seal, such as an air-tight seal, with one reagent reservoir (or one or more, or each) of the plurality of reagent reservoirs to result in a space comprising the outlet open end of one gas injection microchannel (or a corresponding gas injection microchannel) of plurality of gas injection microchannels. The gas-flow control device can be capable of detachably coupling to and/or forming a seal, such as an air-tight seal, with the waste reservoir to result in a space comprising the inlet open end of the waste gas extraction microchannel. The gas-flow control device can be capable of detachably coupling to and/or forming a seal, such as an air-tight seal, with the product reservoir to result in a space comprising the inlet open end of the product gas extraction microchannel In some embodiments, an outlet of a gas injection valve (or an inlet of a gas extraction valve) is open into the space not through a gas injection microchannel (or a gas extraction microchannel).

In some embodiments, the gas-flow control device is attached to and/or forms a seal, such as an air-tight seal with the microfluidic device. The gas-flow control device can be attached to and/or form a seal, such as an air-tight seal, via a silicone pad sandwiched between the gas-flow control device and the microfluidic device. The silicon pad can comprise a plurality of through holes. The through holes can allow gaseous communication of the outlet opening ends of the gas injection microchannels with reservoirs, such as reagent reservoirs. The through holes can allow gaseous communication of the inlet opening end of a gas extraction microchannel with the waste reservoir. The through holes can allow gaseous communication of the inlet opening end of a gas extraction microchannel with the product reservoir. When the silicon pad is aligned with and sandwiched between the gas-flow control device and the microfluidic device, a plurality of through holes at positions corresponding to the positions of the outlet opening ends of the gas injection microchannels and the inlet opening ends of the gas extraction microchannels.

In some embodiments, one, one or more, or each of the plurality of gas injection microchannels is in gaseous communication with one of the plurality of reagent reservoirs. The outlet open end of one, one or more, or each of the plurality of gas injection microchannels can open to one of the plurality of reagent reservoirs. The waste gas extraction microchannel can be in gaseous communication with the waste reservoir. The inlet open end of the waste gas extraction microchannel can be open to the waste reservoir. The product gas extraction microchannel can be in gaseous communication with the product reservoir. The inlet open end of the product gas extraction microchannel can be open to the product reservoir.

In some embodiments, the reagent exchange gas injection microchannel is in gaseous communication with the reagent exchange reservoir. The outlet open end of the reagent exchange gas injection microchannel can be open to the reagent exchange reservoir. The reagent exchange gas extraction microchannel can be in gaseous communication with the reagent exchange reservoir. The inlet open end of the reagent exchange gas extraction microchannel can be open to the reagent exchange reservoir.

In some embodiments, when a driving gas exits (e.g., pushed into) the outlet of the gas injection microchannel into the reagent reservoir, a reagent in the reagent reservoir is driven (e.g., pushed) from the reagent reservoir through a fluid microchannel of the plurality of fluid microchannels into the reagent exchange reservoir. When a gas exits (e.g., sucked) the inlet of the waste gas extraction microchannel, one or more reagents in the reagent exchange reservoir can be pulled from the reagent exchange reservoir into the reaction chamber then into the waste reservoir. When a gas exits the inlet of the product gas extraction microchannel, one or more reagents in the reagent exchange reservoir can be pulled from the reagent exchange reservoir into the reaction chamber then into the product reservoir

In some embodiments, when a driving gas exits (e.g., pushed into) the outlet of the gas injection microchannel into the reagent reservoir, (i) a reagent in the reagent reservoir is driven (e.g., pushed) from the reagent reservoir through a fluid microchannel of the plurality of fluid microchannels into the reagent exchange reservoir, and (ii) a gas in the reagent exchange reservoir exits the reagent exchange reservoir. When a driving gas exits (e.g., pushed into) the outlet of the reagent exchange gas injection microchannel into the reagent exchange reservoir, one or more reagents in the reagent exchange reservoir can be driven from the reagent exchange reservoir into the reaction chamber. Alternatively or additionally, when a gas exits the inlet of the waste gas extraction microchannel, one or more reagents in the reagent exchange reservoir can be pulled from the reagent exchange reservoir into the reaction chamber then into the waste reservoir. Alternatively or additionally, when a gas exits the inlet of the product gas extraction microchannel, one or more reagents in the reagent exchange reservoir can be pulled from the reagent exchange reservoir into the reaction chamber then into the product reservoir. The one or more reagents in the reagent exchange reservoir can be mixed in the reagent exchange reservoir or the mixing reservoir.

In some embodiments, when a gas exits (e.g., sucked or pulled) the inlet of the waste gas extraction microchannel from the waste reservoir, a waste in the reaction chamber is pulled (or sucked) from the reaction chamber through the waste fluid microchannel into the waste reservoir. When a gas exits the inlet of the product gas extraction microchannel from the product reservoir, a product in the reaction chamber can be pulled from the reaction chamber through the product fluid microchannel into the product reservoir, optionally wherein the product is generated using at least one reagent.

In some embodiments, when the gas injection microchannel is under a positive pressure and/or the gas injection valve is in an open state and/or (ii) the waste gas extraction microchannel is under a negative pressure and/or the waste gas extraction valve is in an open state, (1) a reagent in the reagent reservoir is driven (e.g., pushed) through a fluid microchannel of the plurality of fluid microchannels into the reagent exchange reservoir then into the reaction chamber, and/or (2) a waste generated in the reaction chamber from the reagent is driven (e.g., sucked or pulled) from the reaction chamber through the waste fluid microchannel into the waste reservoir.

In some embodiments, when the gas injection microchannel is under a positive pressure and/or the gas injection valve is in an open state and/or when the product gas extraction microchannel is under a negative pressure and/or the product gas extraction valve is in an open state, a reagent in the reagent reservoir is driven (e.g., pushed) through a fluid microchannel of the plurality of fluid microchannels into the reagent exchange reservoir then into the reaction chamber, and a product generated in the reaction chamber from the reagent is driven (e.g., pulled or sucked) from the reaction chamber through the product fluid microchannel into the product reservoir.

In some embodiments, when the gas injection microchannel is under a positive pressure and/or the gas injection valve is in an open state, a reagent in the reagent reservoir is driven (e.g., pushed) through a fluid microchannel of the plurality of fluid microchannels into the reagent exchange reservoir. When the waste gas extraction microchannel is under a negative pressure and/or the waste gas extraction valve is in an open state, (1) a reagent in the reagent exchange reservoir can be pulled (or sucked) into the reaction chamber, and/or (2) a waste generated in the reaction chamber from the reagent is pulled from the reaction chamber through the waste fluid microchannel into the waste reservoir. When the product gas extraction microchannel is under a negative pressure and/or the product gas extraction valve is in an open state, (1) a reagent in the reagent exchange reservoir can be pulled into the reaction chamber, and/or (2) a product generated in the reaction chamber from the reagent can be pulled (or sucked) from the reaction chamber through the product fluid microchannel into the product reservoir, optionally wherein the product is generated using the reagent.

In some embodiments, the mixing gas extraction microchannel is under a negative pressure and/or the mixing gas extraction valve is in an open state, two or more reagents in the reagent exchange reservoir can be pulled (or sucked) from the reagent exchange reservoir into the mixing reservoir, thereby mixing the two or more reagents. When the mixing gas injection microchannel is under a positive pressure and/or the mixing gas injection valve is in an open state, the one or more reagents in the mixing reservoir can be driven (e.g., pushed) into the reagent exchange reservoir. When the waste gas extraction microchannel is under a negative pressure and/or the waste gas extraction valve is in an open state, the one or more reagents in the reagent exchange reservoir can be pulled from the reagent exchange reservoir into the reaction chamber where a waste is generated in the reaction chamber from the one or more reagents and the waste is pulled from the reaction chamber through the waste fluid microchannel into the waste reservoir. When the product gas extraction microchannel is under a negative pressure and/or the product gas extraction valve is in an open state, the one or more reagents in the reagent exchange reservoir can be pulled from the reagent exchange reservoir into the reaction chamber where a product is generated using the one or more reagents and the product is pulled into the product reservoir.

Disclosed herein include sample preparation devices (or sample analysis devices). The sample preparation devices can be used for analysis, such as cell analysis, including single cell analysis. In some embodiments, a sample preparation device comprises: a reaction module of the present disclosure. The sample preparation device can comprise a heating element in contact with the microfluidic device of the reaction module. In some embodiments, the microfluidic device is sandwiched between the gas-flow control device and the heating element.

In some embodiments, a sample preparation device comprises: a gas-flow control device disclosed herein. The gas-flow control device can be capable of detachably coupling to and/or forming a seal (e.g., an air-tight seal) with a microfluidic device, or one or more reservoirs thereof disclosed herein. The gas-flow control device can be detachably coupled to and/or form a seal (e.g., an air-tight seal) with a microfluidic device, or one or more reservoirs thereof, disclosed herein. The sample preparation device can comprise a heating element (e.g., a heating block). The heating element can be used to heat the microfluidic device. In some embodiments, the microfluidic device is sandwiched between the gas-flow control device and the heating element when the microfluidic device, the gas-flow control device, and the heating element are in an assembled state. The microfluidic device can be below the gas-flow control device in the assembled state. The heating element can be below the microfluidic device in the assembled state.

In some embodiments, the sample preparation device further comprises an injection pump. The injection pump can provide a gas (e.g., a driving gas) to the plurality of gas injection valves. The sample preparation device can comprise an extraction pump. The extraction pump can provide a suction to the plurality of gas extracting valves. The extraction pump can extract gas through the plurality of extraction valves. The same pump can be the injection pump and the extraction pump. The injection pump and/or the extraction pump can be adjacent the reaction module. The injection pump and/or the extraction pump can be below the reaction module when the sample preparation device is in an upright orientation.

In some embodiments, the sample preparation device further comprises a control unit (or a control module). The control unit can be in electrical communication with the plurality of gas injection valves, the plurality of gas extraction valves, the heating element, the injection pump, and/or the extraction pump. The control unit can control the plurality of gas injection valves, the plurality of gas extraction valves, the heating element, the injection pump, and/or the extraction pump. The control unit can be adjacent the reaction module. The control unit can be below the reaction module when the sample preparation device is in an upright orientation. The control unit can be adjacent the injection pump and/or the extraction pump. In some embodiments, the sample preparation device further comprising a housing (or a platform) to which the gas-flow control device, the heating element, the control unit, the injection pump, and/or the extraction pump are attached (and/or that encloses which the gas-flow control device, the heating element, the control unit, the injection pump, and/or the extraction pump). In some embodiments, a size of the sample preparation device is 10 mm to 100 cm.

Disclosed herein include sample preparation systems (or sample analysis systems). The sample preparation systems can be used for analysis, such as cell analysis, including single cell analysis. In some embodiments, a sample preparation system comprises: at least one gas-flow control device disclosed herein. The sample preparation system can comprise at least one drive module. The drive module can be capable of detachably coupling (e.g., attaching) to a microfluidic device disclosed herein. For example, the microfluidic device can sit on the at least one drive module. The drive module can be capable of detachably coupling (e.g., attaching) to a gas-flow control device. For example, the gas-flow control device can be attached to the at least one drive module.

In some embodiments, sample preparation system wherein the at least one drive module comprises a microfluidic device drive module. The microfluidic device can be reversibly coupled to (e.g., sit on) the microfluidic device drive module. The microfluidic device drive module can move the microfluidic device. The microfluidic device drive module can move the microfluidic device horizontally between an away horizontal position (or a reagent loading position) and a coupling horizontal position (or a contacting horizontal position, a reagent exchange horizontal position, or a reaction horizontal position). When the microfluidic device drive module is in the away horizontal position, the microfluidic device may not be below the gas-flow control module. When the microfluidic device drive module is in the coupling horizontal position, the microfluidic device can be below the gas-flow control device and/or can be detachably coupled (e.g., attached) to and/or forms a seal (e.g., an air-tight seal) with the gas-flow control device. The microfluidic device drive module can comprise at least one sliding table assembly. The sliding table assembly can comprise a sliding table, a sliding table support base, and a stepping motor. The microfluidic device can sit on the sliding table, for example. The at least one drive module can comprise a gas-flow control drive module. The gas-flow control device can be coupled (e.g., attached) to the gas-flow control drive module. The gas-flow control drive module can move the gas-flow control device. The gas-flow control drive module can move the gas-flow control module vertically between an away vertical position and a coupling vertical position (or a contacting vertical position, a reagent exchange vertical position, or a reaction vertical position). When the microfluidic device drive module is in the coupling horizontal position and the gas-flow control drive module is in the away vertical position, the microfluidic device can be below the gas-flow control device. When the microfluidic device drive module is in the coupling horizontal position and the gas-flow control drive module is in the coupling vertical position, the microfluidic device can be detachably coupled to and/or form a seal (e.g., an air-tight seal) with the microfluidic device, or reservoirs thereof. The gas-flow control drive module can comprise at least one push-rod assembly. The push-rod assembly can comprise a drive motor, a gear shaft attached to the drive motor, a slide rail, and a gear rack. The gas-flow control device can be coupled directly or indirectly to the push-rod assembly, or a component thereof (e.g., the gear rack).

In some embodiments, the sample preparation system further comprises a heating element (e.g., a heating block). The heating element can heat the microfluidic device. The heating element can heat the microfluidic device from below and/or can be in contact with the microfluidic device from the below. In some embodiments, the sample preparation system further comprises an injection pump. The injection pump can provide a gas (e.g., drive a gas) to the plurality of gas injection valves. The sample preparation system can further comprise an extraction pump. The extraction pump can provide suction to (or extract gas from or through) the gas extracting valves. A pump can be both the injection pump and the extraction pump. In some embodiments, the sample preparation system further comprises a control unit. The control unit can be in electrical communication the plurality of gas injection valves, the plurality of gas extraction valves, the heating element, the injection pump, the extraction pump, the drive module, the horizontal drive module, and/or the vertical drive module. The control unit can control the plurality of gas injection valves, the plurality of gas extraction valves, the heating element, the injection pump, the extraction pump, the drive module, the horizontal drive module, and/or the vertical drive module. In some embodiments, the sample preparation system further comprises a housing (or a platform). The gas-flow control device, the heating element, the control unit, the injection pump, the extraction pump, the at least one drive module, the microfluidic device drive module, and/or the gas-flow control device drive module can be attached to, fixed to, and/or enclosed in the housing. In some embodiments, the sample preparation system the control unit comprises a control unit interface for controlling and/or programming the control unit, for example, using a computer, a control software, a programmable software, or a combination thereof. The sample preparation system can comprise the computer.

Disclosed herein include methods of sample preparation (or sample analysis) using a microfluidic device. In some embodiments, a method of preparation uses a microfluidic device disclosed herein, a gas-flow control device disclosed herein, a reaction module disclosed herein, a sample preparation device disclosed in, and/or the sample preparation system disclosed herein.

Disclosed herein include methods of reagent loading In some embodiments, a method of reagent loading comprises: (a) providing a microfluidic device of the present disclosure. The method can comprise (b) loading a first reagent and a second reagent into a first reagent reservoir and a second reagent reservoir of the plurality of reagent reservoirs. The method can comprise (c1) flowing the first reagent from the first reagent reservoir into the reagent exchange reservoir through a first fluid microchannel of the plurality fluid microchannels, then into the reaction chamber, and then into the waste reservoir. The method can comprise (c2) flowing the second reagent from the second reagent reservoir into the reagent exchange reservoir chamber through a second fluid microchannel of the plurality fluid microchannels, then into the reaction chamber, and then into the waste reservoir.

In some embodiments, the method further comprises (b2) loading a third reagent into a third reagent reservoir of the plurality of reagent reservoirs. The method can comprise (c3) flowing the third reagent into the into the reagent exchange reservoir through a third fluid microchannel of the plurality fluid microchannels then into the reaction chamber, thereby a reaction occurs in the reaction chamber. The method can comprise (d) flowing one or more reaction products in the reaction chamber into the product reservoir.

In some embodiments, a method of reagent loading comprises: (a) providing the microfluidic device disclosed herein. One, one or more, or each of the plurality of reagent reservoirs can comprise a reagent. The method can comprise (c) sequentially flowing the reagent in the one, one or more, or each of the plurality of reagent reservoirs into the reagent exchange reservoir through a fluid microchannel of the plurality fluid microchannels and then into the reaction chamber. The method can comprise (d) flowing one or more waste products generated in the reaction chamber into the waste reservoir, and/or flowing one or more reaction products in the reaction chamber into the product reservoir.

In some embodiments, the first reagent comprises a plurality of cells. The second reagent can comprise a plurality of particles. One, one or more, or each of the plurality of particles can comprise a plurality of barcode molecules. Thus single cells and single particles can be loaded into microwells of the microwell array. In some embodiments the third reagent comprises a cell lysis reagent, an enzyme, PCR primers, and/or therapeutic compounds. The reaction products can comprise a plurality of barcoded target nucleic acids and/or reverse transcription products. In some embodiments, the reaction comprises cell lysis, ligand-binding, cell-cell interaction, cell capture, nucleic acid synthesis, cellular response to a therapeutic compound, nucleic acid barcoding, reverse transcription, or a combination thereof.

In some embodiments, the microfluidic device is reversibly coupled to a gas-flow control device disclosed herein. Flowing the reagents can comprise flowing the reagents using one or more gas injection valves of the plurality of gas injection valves and one or more gas extraction valves of the plurality of gas extraction valves. The gas-flow control device can be comprised in (e.g., attached to) a reaction module, a sample preparation device, and/or the sample preparation system disclosed herein. Flowing the reagents can comprise controlling the gas injection valves and gas extraction valves using the control unit to flow the reagents.

Disclosed herein include methods of nucleic acid analysis. In some embodiments, a method of nucleic acid analysis comprises generating a plurality of barcoded target nucleic acids using a reagent loading method of the present disclosure. The method can comprise analyzing the plurality of barcoded target nucleic acids. In some embodiments, the analyzing the plurality of barcoded target nucleic acids comprises determining the sequences of the plurality of barcoded target nucleic acids.

Disclosed herein include method of performing a reaction. In some embodiments, a method of performing a reaction comprises: (a) providing a microfluidic device and a gas-flow control device disclosed herein. The method can comprise (b) loading one or more reagents into the plurality of reagent reservoirs. The method can comprise reversibly coupling the microfluidic device and the gas-flow control device. In some embodiments, the method comprises (a) providing a reaction module disclosed herein. For one, one or more, or each reagent reservoir of the plurality of reagent reservoirs loaded with the one or more reagents, the method can comprise performing (c) injecting gas into the reagent reservoir through a gas injection valve of the plurality of gas injection valves and a gas injection microchannel of the plurality of gas injection microchannels, thereby applying a positive gas pressure to the reagent reservoir, thereby injecting the one or more reagents from the reagent reservoir into the reagent exchange reservoir. The method can comprise performing (d1) extracting gas from the waste reservoir through the waste gas extraction valve and the waste gas extraction microchannel, thereby applying a negative gas pressure to the waste reservoir, thereby transferring a reagent from the reagent exchange reservoir to the reaction chamber, wherein a waste (or a reaction waste) is generated; and/or (d2) extracting gas from the product reservoir through the product gas extraction valve and the product gas extraction microchannel, thereby applying a negative gas pressure to the product reservoir, thereby transferring a reagent from the reagent exchange reservoir to the reaction chamber, wherein a product is generated. The method can comprise (e) allowing the one or more reagents in the reaction chamber to react to generate the waste or the product. The method can comprise (f1) extracting gas from the waste reservoir through the waste gas extraction valve and the waste gas extraction microchannel, thereby applying a negative gas pressure to the waste reservoir, thereby extracting the waste from the reaction chamber into the waste reservoir; and/or (f2) extracting gas from the product reservoir through the product gas extraction valve and the product gas extraction microchannel, thereby applying a negative gas pressure to the product reservoir, thereby extracting the product from the reaction chamber into the product reservoir.

In some embodiments, a method of performing a reaction comprises (a1) providing the sample preparation device or the sample preparation system of disclosed herein and one or more microfluidic devices of the present disclosure. The method can comprise (a2) coupling each of the one or more gas-flow control devices to one microfluidic device of the one or more microfluidic devices. The method can comprise (b) loading one or more reagents into the plurality of reagent reservoirs. For each reagent reservoir of the plurality of reagent reservoirs loaded with the one or more reagents, the method can comprise (c) injecting gas into the reagent reservoir through a gas injection valve of the plurality of gas injection valves and a gas injection microchannel of the plurality of gas injection microchannels, thereby applying a positive gas pressure to the reagent reservoir, thereby injecting the one or more reagents from the reagent reservoir into the reagent exchange reservoir. The method can comprise (d1) extracting gas from the waste reservoir through the waste gas extraction valve and the waste gas extraction microchannel, thereby applying a negative gas pressure to the waste reservoir, thereby transferring the one or more reagents from the reagent exchange reservoir to the reaction chamber, wherein a waste (or reaction waste) is performed. The method can comprise (d2) extracting gas from the product reservoir through the product gas extraction valve and the product gas extraction microchannel, thereby applying a negative gas pressure to the product reservoir, thereby transferring the one or more reagents from the reagent exchange reservoir to the reaction chamber, wherein a product (or a reaction product) is formed. The method can comprise (e) allowing the one or more reagents in the reaction chamber to react to generate a waste and/or a product. The method can comprise (f1) extracting gas from the waste reservoir through the waste gas extraction valve and the waste gas extraction microchannel, thereby applying a negative gas pressure to the waste reservoir, thereby extracting the reaction waste from the reaction chamber into the waste reservoir. The method can comprise (f2) extracting gas from the product reservoir through the product gas extraction valve and the product gas extraction microchannel, thereby applying a negative gas pressure to the product reservoir, thereby extracting a product from the reaction chamber into the product reservoir. In some embodiments, coupling the each of the one or more gas-flow control devices to one microfluidic device of the one or more microfluidic devices comprises moving the gas-flow control module and/or moving the reaction module, thereby aligning the gas-flow control module and the reaction module.

### BRIEF DESCRIPTION OF THE DRAWINGS

FIGS. 1A and 1B show a front view and a rear view, respectively, of a representative microfluidic device capable of performing reagent exchange disclosed herein.

FIGS. 2A and 2B show a top view and a bottom view, respectively, of a representative microfluidic device disclosed herein. FIG. 2A shows a reagent exchange unit (e.g., a reagent exchange cartridge). FIG. 2B shows the reagent exchange unit and a reaction unit.

FIG. 3 shows a top view of a reagent exchange unit disclosed herein.

FIG. 4 shows a work flow of the reagent exchange unit shown in FIG. 3

FIGS. 5A and 5B show a top view and a bottom view, respectively, of a representative microfluidic device disclosed herein. FIG. 5A shows a reagent exchange unit (e.g., a reagent exchange cartridge). FIG. 2B shows the reagent exchange unit and a reaction unit.

FIG. 6 shows the work flow of the reagent exchange unit shown in FIGS. 5A and 5B.

FIGS. 7A-7C show a representative liquid reagent exchange process and reaction using a microfluidic device (FIG. 7A) disclosed herein. FIG. 7B illustrates liquid reagent exchange process for different reagents in the reagent exchange reservoir. FIG. 7C shows reaction of different reagents on the reaction unit of the microfluidic device.

FIGS. 8A and 8B show a top view and a bottom view, respectively, of a representative cell reaction plate disclosed herein.

FIGS. 9A and 9B show a top view and a bottom view, respectively, of a representative integrated gas path control plate disclosed herein.

FIGS. 10A and 10 B show a top view and a bottom view, respectively, of a representative integrated gas path control plate.

FIG. 11 is a schematic structural view of a representative cell sample preparation device disclosed herein.

FIG. 12A is a schematic structural top view of a representative cell reaction plate disclosed herein. FIG. 12B is a schematic structural view of an attaching surface between a representative cell reaction plate and a platform disclosed herein.

FIG. 13 is a schematic view of an internal structure of a representative gas-path control board disclosed herein.

FIGS. 14A, 14B, and 14C show schematic structural views of a representative device for preparing a single-cell sample disclosed herein.

FIG. 15A is a three-dimensional structural view of a representative cell reaction plate disclosed herein. FIG. 15B is a bottom structural view of a representative cell reaction plate disclosed herein.

FIG. 16 is a structural view of a representative cell reaction module disclosed herein.

FIG. 17A is a three-dimensional structural view of a representative integrated gas-path control board disclosed herein. FIG. 17B is a bottom structural view of a representative integrated gas-path control board disclosed herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

In recent years, microfluidic devices (such as microfluidic chips) have been developed for new fields of research involving biology, chemistry, medicine, fluid, electronics, materials, machinery and other disciplines. The microfluidic chip technology is a technology which integrates basic operating units such as sample preparation, reaction, separation, and detection in biological, chemical, and medical analysis processes into a micrometer scale chip and automatically completes the whole analysis process.

The structural design of a microfluidic chip requires placing different reagents in the chip for reaction. However, the operation of the microfluidic chip often involves the exchange reaction of many different reagents. At this time, an operator is usually required to add or replace different reagents, or a specialized instrument is needed for operation, where a series of reactions may involve reaction operations of up to tens of different reagents. The process of adding different reagents easily causes waste of redundant reagents, and meanwhile produces problems such as reagent contamination.

Currently, reagents used in microfluidic chips are mostly added by external injection to allow the reagents to flow along pipelines under external force driving, such as a microquantitative sampling structure. The microquantitative sampling structure includes a quantification tube. In use, air is first removed from the quantification tube, and a tiny amount of liquid is then quantitatively sucked from a container to the quantification tube using the principle of negative pressure. Then, the quantification tube is displaced (moved or rotated) to a specified position and the liquid is pushed out of the quantification tube using air to achieve the purpose of sampling.

However, in such a microquantitative sampling structure, quantification and sampling are two separate steps, and displacement is required in sampling after quantification; thus integration into a tiny biochip is difficult or impossible. Meanwhile, this method of pushing the liquid out using air inevitably mixes air in the liquid while pushing the liquid out, so as to bring air into a reactor of the biochip, which affects detection results of samples by the biochip.

Therefore, it remains an urgent technical challenge to provide a microfluidic device with both working performance and the function of automatically exchanging different.

Cells are basic units in biology, and intensive research has been conducted to individually separate, study and compare the cells. Single-cell sequencing refers to sequencing of relatively simple genomes of single-cell microorganisms, as well as larger and more complex human cell genomes in DNA research.

Single-cell sequencing mainly includes single-cell genome sequencing and transcriptome sequencing, respectively showing changes of the genome and transcriptome of a single cell by performing sequence analysis on DNA and RNA within the single cell. Single-cell whole-genome sequencing is to perform non-selective, uniform amplification on all genome sequences of a selected target cell, and then perform high-throughput sequencing using exon capture techniques. Single-cell transcriptome sequencing is cDNA sequencing using high-throughput sequencing techniques to obtain almost all transcripts of a specific organ or tissue in a certain state, and is mainly used for mining gene regulatory networks at a whole-genome scale, and is especially suitable for highly heterogeneous stem cells and cell populations in early embryonic development. Single-cell transcriptome analysis combined with a live cell imaging system is more helpful for in-depth understanding of processes such as cell differentiation, cell reprogramming and transdifferentiation, and relevant gene regulatory networks.

CN208104383U discloses a microfluidic chip for efficient single-cell droplet preparation, including: a reaction liquid inlet; a marker solution inlet; a single-cell inlet, communicating with a cell reservoir, the cell reservoir being provided with an agitation device; an oil phase inlet; a single-cell channel having an inlet communicating with the single-cell inlet; a liquid mixing channel, having an inlet separately communicating with an outlet of the single-cell channel, the reaction liquid inlet, and the marker solution inlet; a droplet generation channel, having an inlet communicating with an outlet of the liquid mixing channel and the oil phase inlet, where an oil phase is wrapped around the surface of a single cell in the droplet generation channel to form single-cell droplets; and a droplet generation outlet, communicating with an outlet of the droplet generation channel.

CN103571738A discloses a microfluidic chip device based on a chemokine enrichment effect and a preparation method thereof. The microfluidic chip device consists of a PDMS substrate layer, two corresponding microfluidic chip modules A and B, a semipermeable membrane arranged between the two microfluidic chip modules, a top cover plate, and corresponding sample inlet and outlet pipelines. The microfluidic chip module A is used to inject, enrich and sort cell samples to be processed, and a sample pool in the module A is connected to two sample inlets and two sample outlets, so as to realize injection of the cell samples to be processed and sorting of tissue stem cells enriched on the semipermeable membrane. The module B is used to inject chemokines so as to form a chemotactic effect in an area of the sample pool of the module A close to the semipermeable membrane by means of the semipermeable membrane. Various tissue stem cells in the cell samples injected into the module A will move toward the semipermeable membrane under the chemotactic effect and be separated from other cells. The separated stem cells and other cell samples are collected through different sample outlets.

CN10811768A discloses an automatic high-throughput single cell capture method based on a droplet microfluidic chip. The microfluidic chip consists of two layers, where the upper layer is a flow path inlet and outlet layer; the lower layer is a flow path control layer; the flow path inlet and outlet layer has a liquid flow path channel inlet and a liquid flow path channel outlet; and the flow path control layer consists of a single cell capture flow path channel, a gas path channel, and a droplet generation unit. In the method, the gas flow path channel with controllable gas pressure is introduced, so that a negative pressure flow path channel can be formed, and the single-cell suspension can be automatically sucked into a capture trap so as to conveniently observe and detect proliferation, differentiation, drug reaction and other behaviors of single cells.

In most gas path systems used in single-cell sample preparation chips currently on the market, syringes are used to connect various elements such as switch valves and pressure sensors. However, the parts need to be converted through adapters and gathered in the conversion process, which easily produces unstable deviations. It remains an urgent unsolved technical problem to integrate the gas path system as a whole and reduce the volume of the parts while ensuring stable performance.

CN208701026U discloses a gene sequencing chip fixing device, a chip platform and a gene sequencer. The gene sequencing chip fixing device comprises a fixed block, a rotating press block, a first pin shaft, a second pin shaft, a third pin shaft, and an elastic element. The fixed block is provided on a platform body of the chip platform. The fixed block comprises a main body part, the rotating press block comprises a hinge part and a fixed part connected to the hinge part, the first pin shaft is mounted on the hinge part, the second pin shaft is mounted on the main body part, the third pin shaft hinges the hinge part to the main body part, and the fixed part is used to press the gene sequencing chip so as to fix the gene sequencing chip on the platform body of the chip platform, one end of the elastic element is fixed on the first pin shaft, and the other end is fixed on the second pin shaft.

CN105199949A discloses a fluid control device for gene sequencing. The fluid control device for gene sequencing comprises a reagent component, a first multiport valve, a first three-port valve, a gene sequencing chip and a drive component. The reagent component is connected to the gene sequencing chip by means of the first multiport valve and the first three-port valve. A first gene sequencing channel and a second gene sequencing channel are arranged in the gene sequencing chip, such that a reagent in the reagent component can automatically flow into the first gene sequencing channel and the second gene sequencing channel to carry out a reaction and to acquire fluorescence images. Moreover, when a fluorescent sequencing reaction is carried out in the first gene sequencing channel, fluorescence image acquisition can be performed in the second gene sequencing channel, thereby effectively reducing the gene sequencing time, and accordingly effectively improving the efficiency of gene sequencing. In this way, gene sequencing costs for the fluid control device for gene sequencing are reduced, and the efficiency of gene sequencing is increased.

CN106010949A discloses a sequencing device. The sequencing device has at least one sequencing channel fluidically connect a first gap with a second gap. The sequencing channel is formed as a cavity in the region of the first gap (108) and is formed as a pore in the region of the second gap, and the pore has a smaller cross section than the cavity.

With the maturity of sequencing technologies, high-throughput single-cell sequencing is gradually gaining attention. A single-cell sequencing technology mainly includes labelling a target cell population of a sample first to label molecular tags of different sequences on one cell, and then sequencing and analyzing the whole sample, to obtain differences in the cell heterogeneity of the samples. This method is widely used in clinical and therapeutic fields. However, such a series of processes including processing a cell sample, labelling a molecular tag, and then reverse transcription require a series of instruments and equipment and a skilled operator to operate in a routine laboratory, and often takes half a day to a day to complete the entire operation process.

Therefore, it is now necessary to improve the existing equipment, improve the automation degree of the equipment, shorten the operation time, and improve efficiency. Currently, automated instruments for high-throughput single-cell preparation on the market only carry out labelling of molecular tags, and have problems such as complex structures, a large footprint and complex operation.

CN107354093A discloses, which includes: a frame having at least two stations; and a cell separation and culture device for separating and culturing cells, wherein the cell separation and culture device is arranged on the frame. The cell preparation equipment is capable of preparing at least two groups of cells at the same time by arranging at least two stations and allowing the cell separation and culture devices to correspond to the stations one by one, which effectively improves the efficiency of cell preparation. Meanwhile, at least two groups of the same cells are prepared so that the deviation between the two groups of cells, which is caused by different equipment, is effectively avoided. At least two groups of the different cells are prepared; no additional equipment needs to be purchased; the quantity of needed equipment is reduced. However, the cell preparation equipment has a complex structure and a large footprint.

CN107636142A discloses an automated cell cultivation device and an operating method of the cultivation device. The cultivation device includes: an incubator for accommodating at least one container for culturing cells; a microscope for observing a status of the cells in the container; a robot arm for moving the container; a liquid handler for allowing liquid to flow into the container or discharging liquid from the container; and a control device for controlling the operation of at least one of the incubator, the microscope, the robot arm and the liquid handler. The movement of the container by the robot arm causes problems such as a complex structure and inconvenient operation.

CN106367343A discloses a fully automatic intelligent cell culture device and a control method thereof. The cell culture device includes a controller, a first regulating mechanism, a second regulating mechanism, a third regulating mechanism, a culture frame, a first control door, a second control door, a first pipetting mechanism, a second pipetting mechanism, a waste liquid collection mechanism, a bottle opening/closing mechanism and a liquid adding mechanism; and at least three cavities, which include a keeping cavity, a transition cavity and a pre-heating cavity, are arranged in a storage cavity. Use of the device is convenient, rapid and precise in liquid replacing and has no influence on cell growth; moreover, the device is convenient for pre-heating, and has a relatively high pre-heating speed. However, there are still problems of a complex structure, inconvenient operation and a large footprint.

The existing cell sample preparation devices all have problems such as complex structures, large footprints, and inconvenient operation. Therefore, there remains an urgent need for cell sample preparation devices that have a simple structure and a small footprint, while still ensuring easy operation, allowing reverse transcription operation of a cell sample, and lowering operating difficulty faced by an operator.

Provided herein include a microfluidic device capable of performing reagent exchange, a gas-flow control device, a cell reaction module, a device for preparing cell samples, such as a single-cell sample, and method of use thereof. The devices disclosed herein may allow high-throughput single-cell sample preparation and barcoding of target nucleic acids.

### Microfluidic Devices

Disclosed herein include embodiments of a microfluidic device. In some embodiments, a microfluidic device comprises a reagent exchange unit. The reagent exchange unit can comprise a plurality of reagent reservoirs and a reagent exchange reservoir on an upper surface of the reagent exchange unit. The microfluidic device can comprise a reaction unit. The microfluidic device can comprise at least one reaction chamber and a plurality of fluid microchannels formed between a lower surface of the reagent exchange unit and a upper surface of reaction unit. The reaction chamber can comprise an inlet and an outlet. In some embodiments, fluid microchannels of the plurality of fluid microchannels connect (i) reagent reservoirs of the plurality of reagent reservoirs and (ii) the reagent exchange reservoir. In some embodiments, the reagent exchange reservoir is connected with the inlet of the reaction chamber.

In some embodiments, a microfluidic device comprises a reagent exchange unit. The reagent exchange unit can comprise a plurality of reagent reservoirs and at least one reagent exchange reservoir. The microfluidic device can comprise a reaction unit. The microfluidic device can comprise a reaction chamber and a plurality of fluid microchannels formed between a surface of the reagent exchange unit and a surface of reaction unit. In some embodiments, each of fluid microchannels of the plurality of fluid microchannels connects (i) a reagent reservoir of the plurality of reagent reservoirs and (ii) the reagent exchange reservoir. In some embodiments, the reagent exchange reservoir is connected with an inlet of the reaction chamber

In some embodiments, a microfluidic device comprises a plurality of reagent reservoirs and at least one reagent exchange reservoir. The microfluidic device can comprise a reaction chamber. The microfluidic device can comprise a plurality of fluid microchannels. Each of fluid microchannels of the plurality of fluid microchannels can connect (i) a reagent reservoir of the plurality of reagent reservoirs and (ii) the reagent exchange reservoir. The reagent exchange reservoir can be connected with an inlet of the reaction chamber.

In some embodiments, a microfluidic device comprises a plurality of reagent reservoirs and at least one reagent exchange reservoir. The microfluidic device can comprise a reaction chamber. The microfluidic device can comprise a plurality of fluid microchannels. Different fluid microchannels of the plurality of fluid microchannels can connect (i) different reagent reservoirs of the plurality of reagent reservoirs and (ii) the reagent exchange reservoir. The reagent exchange reservoir can be in fluid communication with the reaction chamber.

In some embodiments, a microfluidic device comprises a plurality of reservoirs. The microfluidic device can comprise a reaction chamber. The microfluidic device can comprise a plurality of fluid microchannels. Each of the plurality of reservoirs can be connected with at least one other reservoir of the plurality of reservoirs via a fluid microchannel of the plurality of fluid microchannels. At least one reservoir of the plurality of reservoirs can be connected with at least two other reservoirs of the plurality of reservoirs. The at least one reservoir can be in fluid communication with the reaction chamber. For example, a reservoir can be an input (e.g., for reagent injection or reagent exchange) reservoir or output (e.g. for waste removal or product collection) reservoirs.

In some embodiments, a microfluidic device comprises a plurality of reservoirs. The microfluidic device can comprise a reaction chamber. The microfluidic device can comprise a plurality of fluid microchannels. One, one or more, or each of the plurality of reservoirs can be connected with at least one other reservoir of the plurality of reservoirs via a fluid microchannel of the plurality of fluid microchannels and/or the reaction chamber directly or via a fluid microchannel of the plurality of fluid microchannels. Optionally, at least one reservoir of the plurality of reservoirs can be connected with at least two other reservoirs of the plurality of reservoirs.

The number of fluid microchannels can be, be about, be at least, be at least about, be at most, or be at most about, 2, 3, 4, 5, 6, 7, 8, 9, 10, 11, 12, 13, 14, 15, 16, 17, 18, 19, 20, 25, 30, 35, 40, 45, 50, or a number or a range between any two of these values. The number of reservoirs, such as reagent reservoirs, can be, be about, be at least, be at least about, be at most, or be at most about, 2, 3, 4, 5, 6, 7, 8, 9, 10, 11, 12, 13, 14, 15, 16, 17, 18, 19, 20, 25, 30, 35, 40, 45, 50, or a number or a range between any two of these values. The number of reagent reservoirs can be, be about, be at least, be at least about, be at most, or be at most about, 1, 2, 3, 4, 5, or a number or a range between any two of these values.

In some embodiments, the microfluidic device comprises a first layer and a second layer reversibly coupled to each other. In some embodiments, the microfluidic device comprises a first layer and a second layer bonded to each other. The first layer can comprise a plurality of grooves. The second layer can cover the plurality of grooves to form the plurality of fluid microchannels. The first layer can comprise a cavity. The second layer can cover the cavity to form the reaction chamber. In some embodiments, the first layer comprises a first plurality of grooves, the second layer comprises a second plurality of grooves, and the first plurality of grooves and the second plurality of grooves together form the plurality of fluid microchannels

In some embodiments, a microfluidic device comprises a reagent exchange unit and a reaction unit bonded to each other. In some embodiments, a first surface of the reagent exchange unit comprises a plurality of reagent reservoirs, a product reservoir, a waste reservoir, and a reagent exchange reservoir. In some embodiments, all reagent reservoirs of the plurality of reagent reservoirs, the product reservoir, and the waste reservoir are connected to the reagent exchange reservoir through a plurality of fluid microchannels and/or a reaction chamber on a second surface of the reagent exchange unit. In some embodiments, the reaction unit covers the plurality of microchannels and the reaction chamber, and forms, together with the second surface of the reagent exchange unit, the plurality of microchannels and the reaction chamber of the microfluidic device.

In some embodiments, a microfluidic device comprises a reagent exchange unit and a reaction unit bonded to each other. In some embodiments, an upper surface of the reagent exchange unit comprises a plurality of reagent reservoirs, a product reservoir, a waste reservoir, and a reagent exchange reservoir. In some embodiments, all reagent reservoirs of the plurality of reagent reservoirs, the product reservoir, and the waste reservoir are connected to the reagent exchange reservoir through a plurality of fluid microchannels and/or a reaction chamber on a lower surface of the reagent exchange unit and in a recess of the lower surface of the reagent exchange unit. In some embodiments, the recess is connected to the reagent exchange reservoir, the product reservoir, and the waste reservoir. In some embodiments, the reaction unit covers the plurality of microchannels, the reaction chamber, and the recess, and forms, together with the recess and the lower surface of the reagent exchange unit, the plurality of microchannels and the reaction chamber of the microfluidic device.

The microfluidic device may be in a form of a thin plate or a chip. A “microfluidic device” may be referred to as a “microfluidic chip” in the embodiments described herein. A “reaction unit” may be referred to as a “working unit” in the embodiments described herein. A “reaction chamber” may be referred to as a “working section” in the embodiments described herein.

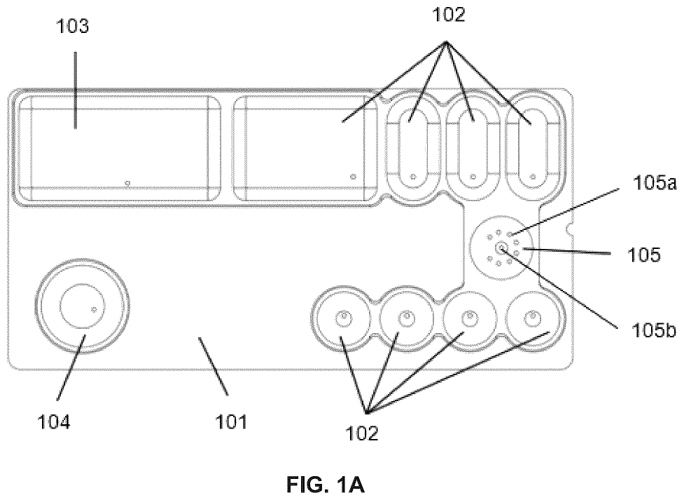

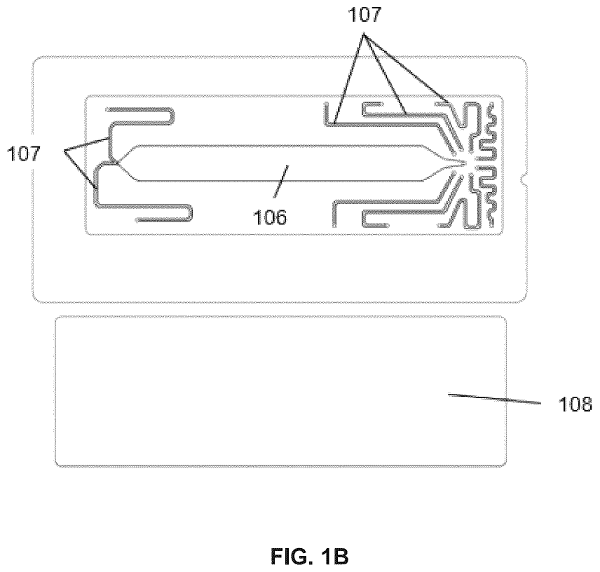

A non-limiting illustration of the present microfluidic device is provided in FIG. 1A and FIG. 1B.

The microfluidic device is an integral disposable chip including a reagent exchange unit 101 and a working unit 108 bonded to each other. The reagent exchange unit may be also referred to as a “top unit” of the microfluidic device. The working unit (or reaction unit) may be also referred to as a “bottom unit” of the microfluidic device. A working unit that includes a microarray may be also referred to as a “microarray unit.”

A left portion of an upper surface of the reagent exchange unit 101 includes a waste reservoir 103 and a product reservoir 104 that are circular and rectangular; and a right portion includes one reagent exchange reservoir 105 and eight reagent reservoirs 102, where four of them are circles of the same size, three of them have an oval shape (with semicircles on two ends and a rectangle in the middle), and the remaining one is a rectangle.

The upper surface of the reagent exchange unit may be also viewed as including an upper side and a lower side, the rectangular waste liquid reservoir 103, the rectangular reagent reservoir 102, and the three reagent reservoirs 102 with oval shape are located on the upper side, and the circular product reservoir 104, the four reagent reservoirs 102 of the same size, and the reagent exchange reservoir 105 are located on the lower side.

The reagent reservoirs 102 are all connected to the reagent exchange reservoir 105 through microchannels 107 (also referred to as fluid microchannels herein), and the reagent exchange reservoir 105, the product reservoir 104, and the waste liquid reservoir 103 are also connected to a working section 106 through microchannels 107.

The working section 106 as shown in FIG. 1B is a space formed between the working unit 108 and the reagent exchange unit 101 after the two units are bonded to each other. A working section may be used for mixing reagents and/or performing a reaction.

In some embodiments, the reagent exchange unit is in direct contact with the reaction unit. The reagent exchange unit and the reaction unit can be bonded to each other. The reagent exchange unit and the reaction unit can form an integral structure. In embodiments, the reagent exchange unit and the reaction unit are bonded to each other to form an integral structure. In some embodiments, the microfluidic device comprises one or more additional layers sandwiched between the reagent exchange unit and the reaction unit. The number of the additional layers is not limited. The reagent exchange unit, the reaction unit, and the one or more additional layers may be bonded together to form an integral structure. The additional layer may, for example, expand (or increase the volume of) the reaction chamber formed between a surface of the reagent exchange unit and a surface of reaction unit.

In some embodiments, the microfluidic device is for single-use, or is disposable after use.

### Reservoirs

The reagent reservoirs can be used to hold or inject reagents. For example, the reagents can be in a form of a solution, a suspension, or an emulsion. The reagents can include particles (such as beads) or cells. The reagents can be loaded into the reagent reservoirs manually or by an instrument or a machine.

The number of the reagent reservoirs are not limited. For example, the number of the reagent reservoirs is, is about, is at least, is at least about, is at most, or is at most about 2, 3, 4, 5, 6, 7, 8, 9, or 10. In some embodiments, the microfluidic device includes 6, 7, 8, 9, or 10 reagent reservoir. In some embodiments, the plurality of reagent reservoirs comprises at least two reagent reservoirs. The fluid microchannels of reagent reservoirs of the plurality of fluid microchannels connecting the plurality of reagent reservoirs to the reagent exchange reservoirs can comprises at least two fluid microchannels. The number of the reagent reservoirs and the number of the fluidic microchannels connecting the plurality of reagent reservoirs to the reagent exchange reservoir can be identical, such as 2, 3, 4, 5, 6, 7, 8, 9, 10 or more.

In some embodiments, one, one or more, or each of the plurality of reagent reservoirs comprises a reagent. Two of the plurality of reagent reservoirs can comprise same or different reagents. In some embodiments, two of the plurality of reagent reservoirs comprises different reagents. In some embodiments, each of the plurality of reagent reservoirs comprises different reagents. In some embodiments, two or more of the plurality of reagent reservoirs comprise an identical reagent. In some embodiments, two of the plurality of reagent reservoirs comprise an identical reagent.

The reagent reservoir can be connected to the reagent exchange reservoir by the fluid microchannels. For example, one or more reagent reservoirs of the plurality of reagent reservoirs, the waste reservoir, and/or the product reservoir each can comprise an opening that connects the reservoir to a fluid microchannel of the plurality of fluid microchannels (e.g., FIG. 1A, circular holes inside 102, 103, 104). Further, the reagent exchange reservoir can comprise one or more openings that connect the reagent exchange reservoir to one or more fluid microchannels of the plurality of fluid microchannels (e.g., FIG. 1A, holes 105a). The number of openings can be, be about, be at least, be at least about, be at most, or be at most about, 2, 3, 4, 5, 6, 7, 8, 9, 10, 11, 12, 13, 14, 15, 16, 17, 18, 19, 20, 25, 30, 35, 40, 45, 50, or a number or a range between any two of these values. For example, a microfluidic device can have 9 reagent reservoirs. The reagent exchange reservoir can have 10 holes, 9 holes for connecting the reagent exchange reservoir to the 9 reagent reservoirs through fluid microchannels and 1 hole for connecting the reagent exchange reservoir to the reaction chamber through the inlet of the reaction chamber. In addition, the reagent exchange reservoir can comprise an opening that connects the reagent exchange reservoir to the inlet of the reaction chamber (e.g., FIG. 1A, center hole 105b).

The waste reservoir can hold waste (such as unwanted liquid, solvent, or aqueous carrier) generated after loading a reagent or after a reaction. The product reservoir can be used to isolate a product from a reaction (such as nucleic acid products from a cell reaction). The waste reservoir and the product reservoir each can be connected to the reaction chamber by the fluid microchannels.

In some embodiments, the reagent exchange unit comprises a waste reservoir on the upper surface of the reagent exchange unit (e.g., on the same surface as the reagent reservoirs). A waste fluid microchannel of the plurality of fluid microchannels can connect the waste reservoir and the outlet of the reaction chamber. Optionally, the waste fluid microchannel can directly connect the waste reservoir and the outlet of the reaction chamber.

In some embodiments, the reagent exchange unit further comprises a product reservoir on the upper surface of the reagent exchange unit (e.g., on the same surface as the reagent reservoirs and the waste reservoir). A product fluid microchannel of the plurality of fluid microchannels can connects the product reservoir and the outlet of the reaction chamber. Optionally, the product fluid microchannel directly connects the product reservoir and the outlet of the reaction chamber.

In some embodiments, the waste fluid microchannel, the product fluid microchannel, and the outlet of the reaction chamber connect at a junction (FIG. 1B). Alternatively, the waste fluid microchannel and the product fluid microchannel can merge into a single fluid microchannel which is connected to the outlet of the reaction chamber.

In some embodiments, the plurality of reagent reservoirs comprises a mixing reservoir. A mixing fluid microchannel of the plurality of fluid microchannels can connect the mixing reservoir and the reagent exchange reservoir. Optionally, the mixing fluid microchannel can split into two or more fluid microchannels which merge into a single fluid microchannel. Optionally, a first portion of the mixing fluid microchannel can connect the mixing reservoir and a mixing chamber and a second portion of the mixing fluid microchannel can connect the mixing chamber and the reagent exchange reservoir.

In some embodiments, one or more reagent reservoirs of the plurality of reagent reservoirs, the reagent exchange reservoir, the waste reservoir, and/or the product reservoir each is formed by a wall protruding from the upper surface of the reagent exchange unit. In some embodiments, each of the reservoirs comprises a tapered bottom surface. In some embodiments, each of the reservoirs comprises a rounded bottom surface. In some embodiments, each of the reservoirs comprises a tapered and rounded bottom surface (e.g., FIG. 1A, 104 and 102 on the lower side). The tapered bottom surface and/or the rounded bottom surface, or a portion thereof, can be higher than (e.g., protruding from), lower than (protruding into), or on the same level as the upper surface of the reagent exchange unit. Optionally, the tapered bottom surface and/or the rounded bottom surface, or a portion thereof, can be disposed in or protrudes into the upper surface of the reagent exchange unit.

The arrangement of the reservoirs on the reagent exchange unit is not limited. In some embodiments, the upper surface of the reagent exchange unit is divided into a first functional area and a second functional area. For example, the first functional area can comprise the product reservoir and the waste reservoir. For example, the second functional area can comprise at least two reagent reservoirs. Optionally the second functional area comprises the reagent exchange reservoir. In some embodiments, the reagent exchange unit is divided into a first functional area that include the product reservoir and the waste reservoir (e.g., FIG. 1A, 103 and 104 on the left side) and a second functional area that includes a plurality of reagent exchange reservoirs and the reagent exchange reservoir (e.g., FIG. 1A, 102 and 105 on the right side).

### Reaction Chamber and Fluid Microchannels

The reaction chamber can comprise an inlet for receiving reagent (e.g., from the reagent exchange reservoir) and an outlet for discharging a waste or product (e.g., to the waste reservoir and the product reservoir, respectively). The reaction chamber can be formed between a surface of the reagent exchange unit and a surface of reaction unit. For example, the reaction chamber can be formed between a lower surface of the reagent exchange unit and a upper surface of reaction unit.

In some embodiments, the reaction chamber comprises two tapered ends forming the inlet and the outlet of the reaction chamber (e.g., FIG. 1A, 106).

In reaction chamber may include additional surfaces, compartments, or structures for performing a reaction (such as a reaction for preparing a cell sample). In some embodiments, the reaction chamber comprises a microwell array, which may comprise at least 100 microwells. For example, the microwell array can be disposed on the upper surface of the reaction unit. In some embodiments, the reaction chamber comprises a microwell array comprising at least 100 microwells, and the microwell array is disposed on the upper surface of the reaction unit.

In some embodiments, to facilitate the reaction, the reaction unit can be figured to receive heat, or can be capable of receiving heat by contacting a heating element. In some embodiments, the lower surface of the reaction unit is capable of being in thermal contact with a heating element. For example, a reaction may be carried out in a microwell array in the reaction chamber on the upper surface of the reaction unit, and the lower surface of the reaction unit is capable of being in thermal contact with a heating element to receive heat.

The reaction chamber may be defined by a feature of the lower surface and/or a feature of the upper surface of the reaction unit. Such features include, but are not limited to recess, protrusion, curvature, slope, and pattern on the surfaces. In some embodiments, the plurality of microchannels and/or the reaction chamber is in a recess of the lower surface of the reagent exchange unit. In some embodiments, the reaction unit covers the plurality of microchannels, the reaction chamber, and the recess, and forms, together with the recess and/or the lower surface of the reagent exchange unit, the plurality of microchannels and the reaction chamber of the microfluidic device. In some embodiments, the plurality of microchannels and/or the reaction chamber is in a recess of the lower surface of the reagent exchange unit, and the reaction unit covers the plurality of microchannels, the reaction chamber, and the recess, and forms, together with the recess and/or the lower surface of the reagent exchange unit, the plurality of microchannels and the reaction chamber of the microfluidic device.

The reaction chamber and/or the fluid microchannels can be formed as a part of the structure of the reagent exchange unit and/or the reaction unit. For example, the reaction chamber can be formed between a lower surface of the reagent exchange unit (e.g., with a recess) and a upper surface of reaction unit (e.g., with a microwell array). Further, the fluid microchannels can be formed in the reagent exchange unit, for example, by casting, drilling, or additive manufacturing. Alternatively, the lower surface of the reagent exchange unit and/or the upper surface of reaction unit may include grooves that form the fluid microchannels when, for example, the lower surface of the reagent exchange unit is in direct contact with the upper surface of reaction unit

In some embodiments, the reagent exchange unit and/or the reaction unit comprises (i) the reaction chamber, or a portion thereof. In some embodiments, the reagent exchange unit and/or the reaction unit comprises (ii) the plurality of fluid microchannels, or a portion of each of one or more fluid microchannels of the plurality of fluid microchannels. In some embodiments, the reagent exchange unit and/or the reaction unit comprises (i) the reaction chamber, or a portion thereof and (ii) the plurality of fluid microchannels, or a portion of each of one or more fluid microchannels of the plurality of fluid microchannels.

In some embodiments, the lower surface of the reagent exchange unit and/or the upper surface of the reaction unit comprises (i) the reaction chamber, or a portion thereof. In some embodiments, the lower surface of the reagent exchange unit and/or the upper surface of the reaction unit comprises (ii) the plurality of fluid microchannels, or a portion of each of one or more fluid microchannels of the plurality of fluid microchannels. In some embodiments, the lower surface of the reagent exchange unit and/or the upper surface of the reaction unit comprises (i) the reaction chamber, or a portion thereof and (ii) the plurality of fluid microchannels, or a portion of each of one or more fluid microchannels of the plurality of fluid microchannels.

### Shapes and Sizes

Suitable shapes and sizes of any one of the microfluidic device, the reagent exchange unit, the reaction unit, the reagent reservoirs, the reagent exchange reservoir, the waste reservoir, the product reservoir, the reaction chamber, and the fluid microchannels are not limited. A shape, such as a cross-sectional shape, can be a circle, a rectangle, an ellipse, a semicircle, a trapezoid, or a combination thereof (e.g., a long oval, oval rectangle, or rounded rectangle). A size can be, for example, width, length, depth (or height), radius, diameter, or circumference.

As described herein, a size of a device (e.g., a microfluidic device, a gas-flow control device, or a sample preparation device), a unit, a reservoir, a chamber, a microchannel, a module (e.g., a reaction module), or a system (e.g., a reaction system or a sample preparation system) can be characterized by measuring any one of a width, a diameter, a height (or depth), a radius, a circumference, or a combination thereof. A can be, for example 1 mm to 2 m. For example, the size is, is about, is at least, is at least about, is at most, or is at most about 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 6 mm, 7 mm, 8 mm, mm, 1 cm, 2 cm, 3 cm, 4 cm, 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, or 20 cm, 30 cm, 40 cm, 50 cm, 60 cm, 70 cm, 80 cm, 90 cm, 1 m, 1.1 m, 1.2 m, 1.3 m, 1.4 m, 1.5 m, 1.6 m, 1.7 m, 1.8 m, 1.9 m, 2 m, or a number or a range between any two of these values.

In some embodiments, a size (e.g., width, length, depth (or height), radius, diameter, or circumference) of the product reservoir, a size of the waste reservoir, a size of one, one or more, of each of the plurality of reagent reservoirs, a size of the reagent exchange reservoir, a size of the reaction chamber, a size of the microfluidic device, a size of the reagent exchange unit, and/or a size of the reaction unit is 1 mm to 20 cm. For example, the size is, is about, is at least, is at least about, is at most, or is at most about 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 6 mm, 7 mm, 8 mm, 9 mm, 1 cm, 2 cm, 3 cm, 4 cm, 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, or 20 cm, or a number or a range between any two of these values.

In some embodiments, a cross-sectional shape of one, one or more, or each of the plurality of fluid microchannels is a circle, a rectangle, an ellipse, a semicircle, a trapezoid, or a combination thereof. In some embodiments, a cross-sectional shape of one, one or more, or each of the plurality of fluid microchannels is a circle. In some embodiments, cross-sectional shape of each of the plurality of fluid microchannels is a circle.

In some embodiments, a size (e.g., width, length, depth (or height), radius, diameter, or circumference) of one, one or more, or each of the plurality of fluid microchannels is 1 mm to 20 cm. For example, the size of one, one or more, or each of the plurality of fluid microchannels is, is about, is at least, is at least about, is at most, or is at most about 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 6 mm, 7 mm, 8 mm, 9 mm, 1 cm, 2 cm, 3 cm, 4 cm, 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, or 20 cm, or a number or a range between any two of these values.

The cross-sectional shapes of the reagent reservoirs can be a circle, a rectangle, an ellipse, a semicircle, a trapezoid, an oval, an oval rectangle, a rounded rectangle, or a combination thereof. In some embodiments, a cross-sectional shape of one, one or more, of each of the plurality of reagent reservoirs is a circle, a rectangle, an ellipse, a semicircle, a trapezoid, or a combination thereof. In some embodiments, a cross-sectional shape of one, one or more, of each of the plurality of reagent reservoirs is a circle or a rectangle. The cross-sectional shapes of different reagent reservoirs can be identical or different. In some embodiments, 2, 3, 4, or 5 different reagent reservoirs can have an identical cross-sectional shape. In some embodiments, a first group of 2, 3, 4, or 5 different reagents reservoirs can have a first identical cross-sectional shape and a second group of 2, 3, 4, or 5 different reagents reservoirs can have a second identical cross-sectional shape.

In some embodiments, the cross-sectional shape of one, one or more, of each of the plurality of reagent reservoirs is modified by one or more additional shapes, such as a partial rectangular shape protruding from a circular cross-sectional shape.

In some embodiments, the height of the each reagent reservoir is, is about, is at least, is at least about, is at most, or is at most about 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 6 mm, 7 mm, 8 mm, 9 mm, 10 mm, 11 mm, 12 mm, 13 mm, 14 mm, or 15 mm, or a number or a range between any two of these values. For example, the height of each reagent reservoirs is any value from 0.1 mm to 10 mm. The heights of different reagent reservoirs can be identical or different. In some embodiments. 2, 3, 4, or 5 different reagent reservoirs can have an identical height. In some embodiments, a first group of 2, 3, 4, or 5 different reagents reservoirs can have a first identical height and a second group of 2, 3, 4, or 5 different reagents reservoirs can have a second identical height.

In some embodiments, the volume of each reagent reservoir is, is about, is at least, is at least about, is at most, or is at most about 0.1 mL, 0.2 mL, 0.3 mL, 0.4 mL, 0.5 mL, 0.6 mL, 0.7 mL, 0.8 mL, 0.9 mL, 1 mL, 1.5 mL, 2 mL, 2.5 mL, 3 mL, 3.5 mL, 4 mL, 4.5 mL, 5 mL, 6 mL, 7 mL, 8 mL, 9 mL, or 10 mL, or a number or a range between any two of these values. For example, the volume of each reagent reservoirs is any value from 0.1 mm to 5 mm. The volumes of different reagent reservoirs can be identical or different. In some embodiments, 2, 3, 4, or 5 different reagent reservoirs can have an identical volume. In some embodiments, a first group of 2, 3, 4, or 5 different reagents reservoirs can have a first identical volume and a second group of 2, 3, 4, or 5 different reagents reservoirs can have a second identical volume.

In some embodiments, 2, 3, 4, or 5 different reagent reservoirs can have an identical cross-sectional shape, an identical height, and an identical volume. In some embodiments, a first group of 2, 3, 4, or 5 different reagents reservoirs can have a first identical cross-sectional shape, a first identical height, and a first identical volume, and a second group of 2, 3, 4, or 5 different reagents reservoirs can have a second identical cross-sectional shape, a second identical height, a second identical volume.

The cross-sectional shape of the reagent exchange reservoirs can be a circle, a rectangle, an ellipse, a semicircle, a trapezoid, an oval, an oval rectangle, a rounded rectangle, or a combination thereof. In some embodiments, a cross-sectional shape of the reagent exchange reservoir is a circle, a rectangle, an ellipse, a semicircle, a trapezoid, or a combination thereof. In some embodiments, a cross-sectional shape of the reagent exchange reservoir is a circle.

In some embodiments, the height of the reagent exchange reservoir is, is about, is at least, is at least about, is at most, or is at most about 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 6 mm, 7 mm, 8 mm, 9 mm, 10 mm, 11 mm, 120 mm, 13 mm, 14 mm, or 15 mm, or a number or a range between any two of these values. For example, the height of the reagent exchange reservoir is any value from 0.1 mm to 10 mm.

In some embodiments, the volume of the reagent exchange reservoir is, is about, is at least, is at least about, is at most, or is at most about 0.1 mL, 0.2 mL, 0.3 mL, 0.4 mL, 0.5 mL, 0.6 mL, 0.7 mL, 0.8 mL, 0.9 mL, 1 mL, 2 mL, 3 mL, 4 mL, 5 mL, 6 mL, 7 mL, 8 mL, 9 mL, 10 mL, 11 mL, 12 mL, 13 mL, 14 mL, or 15 mL or a number or a range between any two of these values. For example, the volume of the reagent exchange reservoir is any value from 0.1 mL to 10 mL.

The cross-sectional shape of the waste liquid reservoirs can be a circle, a rectangle, an ellipse, a semicircle, a trapezoid, an oval, an oval rectangle, a rounded rectangle, or a combination thereof. In some embodiments, a cross-sectional shape of the waste liquid reservoir is a rectangle, a circle, an ellipse, or a combination thereof. In some embodiments, a cross-sectional shape of the waste liquid reservoir is a rectangle.

In some embodiments, the height of the waste liquid reservoir is, is about, is at least, is at least about, is at most, or is at most about 1 mm, 2 mm, 3 mm, 4 mm, 5 mm, 6 mm, 7 mm, 8 mm, 9 mm, or 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, or 25 mm or a number or a range between any two of these values. For example, the height of the waste liquid reservoir is any value from 5 mm to 20 mm.

In some embodiments, the volume of the waste liquid reservoir is, is about, is at least, is at least about, is at most, or is at most about 0.1 mL, 0.2 mL, 0.3 mL, 0.4 mL, 0.5 mL, 1 mL, 2 mL, 3 mL, 4 mL, 5 mL, 6 mL, 7 mL, 8 mL, 9 mL, 10 mL, 15 mL, 20 mL, 25 mL, 30 mL, 35 mL, or 40 mL, or a number or a range between any two of these values. For example, the volume of the waste liquid reservoir is any value from 0.1 mL to 10 mL.

In some embodiments, the cross-sectional shape of the waste reservoir is modified by one or more additional shapes, such as a partial rectangular shape protruding from a circular cross-sectional shape.

The cross-sectional shape of the product reservoir (or the waste reservoir) can be a circle, a rectangle, an ellipse, a semicircle, a trapezoid, an oval, an oval rectangle, a rounded rectangle, or a combination thereof. In some embodiments, a cross-sectional shape of the product reservoir (or the waste reservoir) is a rectangle, a circle, an ellipse, a semicircle, a trapezoid, or a combination thereof. In some embodiments, a cross-sectional shape of the product reservoir (or waste reservoir) is a rectangle or a circle.

In some embodiments, a cross-sectional shape of the product reservoir and a cross-sectional shape of the waste reservoir are identical. In some embodiments, a cross-sectional shape of the product reservoir and a cross-sectional shape of the waste reservoir are different.

In some embodiments, the height of the product reservoir is, is about, is at least, is at least about, is at most, or is at most about 1 mm, 2 mm, 3 mm, 4 mm, 5 mm, 6 mm, 7 mm, 8 mm, 9 mm, or 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, or 25 mm or a number or a range between any two of these values. For example, the height of the product reservoir is any value from 5 mm to 20 mm.

In some embodiments, the volume of the product reservoir is, is about, is at least, is at least about, is at most, or is at most about 0.1 mL, 0.2 mL, 0.3 mL, 0.4 mL, 0.5 mL, 1 mL, 2 mL, 3 mL, 4 mL, 5 mL, 6 mL, 7 mL, 8 mL, 9 mL, 10 mL, 15 mL, 20 mL, 25 mL, 30 mL, 35 mL, or 40 mL, or a number or a range between any two of these values. For example, the volume of the product reservoir is any value from 0.1 mL to 10 mL.

In some embodiments, the cross-sectional shape of the product reservoir is modified by one or more additional shapes, such as a partial rectangular shape protruding from a circular cross-sectional shape.

In some embodiments, the heights of the reagent reservoir, the reagent exchange reservoir, the waste reservoir, and the product reservoir are identical.

The cross-sectional shape of the reaction chamber can be a circle, a rectangle, an ellipse, a semicircle, a trapezoid, an oval, an oval rectangle, a rounded rectangle, or a combination thereof. In some embodiments, a cross-sectional shape of the reaction chamber is a circle, a rectangle, an ellipse, a semicircle, a trapezoid, or a combination thereof. In some embodiments, a shape of the reagent exchange unit is a circle, a rectangle, an ellipse, a semicircle, a trapezoid, or a combination thereof. In some embodiments, a shape of the reagent exchange unit is a rectangle. In some embodiments, a shape of the reaction unit is a circle, a rectangle, an ellipse, a semicircle, a trapezoid, or a combination thereof. In some embodiments, a shape of the reaction unit is a rectangle. In some embodiments, a shape of the microfluidic chip is a circle, a rectangle, an ellipse, a semicircle, a trapezoid, or a combination thereof. In some embodiments, a shape of the microfluidic device is a rectangle.

With the microfluidic chip provided in this embodiment, all reagents needed during the experiment can be stored on the chip, so that cross-contamination during reagent transfer can be effectively avoided. Meanwhile, because of the pipeline design of microchannels, the risk of contamination of pipelines is avoided, and mixed blowing (e.g., transfer of reagents by pressurization or depressurization through microchannels) can be performed according to requirements of the experiment and a continuous process of reagent sample drawing or injection is realized.

### Material

In some embodiments, a device of the present disclosure (e.g., a microfluidic device or a gas-flow control device) or a component thereof (e.g., a plate or platform of a gas-flow control device) can be formed from a material selected from the group consisting of silicon, glass, ceramic, elastomers such as polydimethylsiloxane (PDMS) and thermoset polyester, thermoplastic polymers such as polystyrene, polycarbonate, poly(methyl methacrylate) (PMMA), poly-ethylene glycol diacrylate (PEGDA), Teflon, polyurethane, composite materials such as cyclic-olefin copolymer, and combinations thereof.

### Gas-Flow Control Device

Disclosed herein include embodiments of a gas-flow control device. In some embodiments, a gas-flow control device comprises a plate (such as a board). The gas-flow control device can comprise a plurality of gas injection valves disposed on and in the plate. The gas-flow control device can comprise a plurality of gas injection microchannels disposed in the plate. Each of the plurality of gas injection microchannels can have an outlet open end on a lower surface of the plate. Each of the plurality of gas injection microchannels can be connected with one injection valve of the plurality of injection valves.

The gas-flow control device may be configured to inject a reagent into a reaction, for example, by applying a positive pressure using a gas injection valve. The gas injection valve can, for example, inject a gas into a microchannel to create a positive pressure. In some embodiments, the plurality of gas injection valves comprises a plurality of reagent gas injection valves. In some embodiments, the plurality of gas injection microchannels comprises a plurality of reagent gas injection microchannels.

The gas-flow control device may be configured to extract a waste and/or a product from a reaction, for example, by applying a negative pressure (e.g., suction) using a gas extraction valve. The gas extraction valve can, for example, extract a gas from a microchannel to create a negative pressure. In some embodiments, the gas-flow control device further comprises a plurality of gas extraction valves disposed on and in the plate. In some embodiments, the gas-flow control device further comprises a plurality of gas extraction microchannels disposed in the plate and having an inlet open end on the lower surface of the plate. Each of the plurality of gas extraction microchannels can be connected with a gas extraction vale of the plurality of gas extraction valves.

The gas-flow control device may include independent gas extraction valves to extract a waste and a product independently from a reaction. In some embodiments, the plurality of gas extraction valves comprises a product gas extraction valve and/or a waste gas extraction valve. The plurality of gas extraction microchannels can comprise a product gas extraction microchannel and/or a waste gas extraction microchannel. The product gas extraction microchannel can be connected with the product gas extraction valve. The product gas extraction valve and the product gas extraction microchannel can be used to generate a negative pressure in the product reservoir, which can result in one or more reagents in the reagent exchange reservoir to flow from the reagent exchange reservoir into the reaction chamber, then into the product reservoir. The waste gas extraction microchannel can be connected with the waste gas extraction valve. The waste gas extraction valve and the waste gas extraction microchannel can be used to generate a negative pressure in the waste reservoir, which can result in one or more reagents to flow from the reagent exchange reservoir into the reaction chamber, then into the waste reservoir.

The gas-flow control device may be configured to allow for mixing multiple reagents prior to injecting the mixed reagents into a reaction. For example, the gas-flow control device may be capable of performing reagent exchange, thereby achieving mixing of multiple reagents. In some embodiments, the plurality of gas extraction valves comprises a reagent exchange gas extraction valve, and the plurality of gas extraction microchannels comprises a reagent exchange gas extraction microchannel. In some embodiments, the plurality of gas injection valves comprises a reagent exchange gas injection valve, and the plurality of gas injection microchannels comprises a reagent exchange gas injection microchannel. In some embodiments, the plurality of gas extraction valves comprises a reagent exchange gas extraction valve, the plurality of gas extraction microchannels comprises a reagent exchange gas extraction microchannel, the plurality of gas injection valves comprises a reagent exchange gas injection valve, and the plurality of gas injection microchannels comprises a reagent exchange gas injection microchannel.

In some embodiments, a gas-flow control device comprises a plurality of gas injection valves and a plurality of gas extraction valves disposed on and in a plate (such as a board) of the gas-flow device. The gas-flow control device can comprise a plurality of gas injection microchannels disposed in the plate. Each of the plurality of gas injection microchannels can have an outlet open end on a lower surface of the plate and an inlet open end connected with one injection valve of the plurality of injection valves. The gas-flow control device can comprise a plurality of gas extraction microchannels disposed in the plate. Each of the plurality of gas extraction microchannels can have an inlet open end on the lower surface of the plate. An outlet open end of a waste gas extraction microchannel and an outlet open end of a product gas extraction microchannel can be connected to a waste gas extraction valve and a product gas extraction valve, respectively, of the plurality of gas extraction valves.

In some embodiment, a gas-flow control device comprises a plurality of gas injection valves and a plurality of gas extraction valves disposed on and in a plate (such as a board) of the gas-flow device. The gas-flow control device can comprise a plurality of gas injection microchannels disposed in the plate. Each of the plurality of gas injection microchannels can have an outlet open end on a lower surface of the plate and an inlet open end connected with one injection valve of the plurality of injection valves. The gas-flow control device can comprise a plurality of gas extraction microchannels disposed in the plate. Each of the plurality of gas extraction microchannels can have an inlet open end on the lower surface of the plate and an outlet open end connected to a gas extraction valve of the plurality of gas extraction valves.

In some embodiment, a gas-flow control device comprises a plurality of gas injection microchannels disposed in a plate (such as a board) of the gas-flow control device. Each of the plurality of gas injection microchannels can have an outlet open end on a lower surface of the plate and an inlet open end for connecting to one injection valve of a plurality of injection valves. The gas-flow control device can comprise a plurality of gas extraction microchannels disposed in the plate. Each of the plurality of gas extraction microchannels can have an inlet open end on the lower surface of the plate and an outlet open end for connecting to a gas extraction valve of a plurality of gas extraction valves.

In some embodiment, a gas-flow control device comprises a plurality of gas injection microchannels disposed in a plate (such as a board) of the gas-flow control device. Each of the plurality of gas injection microchannels can have an outlet open end on a lower surface of the plate and an inlet open end disposed within the plate. The gas-flow control device can comprise a plurality of gas extraction microchannels disposed in the plate. Each of the plurality of gas extraction microchannels can have an inlet open end on the lower surface of the plate and an outlet open end for connecting to a gas extraction valve disposed within the plate.

The number of gas valves (e.g., gas injection valves or gas extraction valves) can be, be about, be at least, be at least about, be at most, or be at most about, 1, 2, 3, 4, 5, 6, 7, 8, 9, 10, 11, 12, 13, 14, 15, 16, 17, 18, 19, 20, 25, 30, 35, 40, 45, 50, or a number or a range between any two of these values. The number of gas microchannels (e.g., gas injection microchannels or gas extraction microchannels) can be, be about, be at least, be at least about, be at most, or be at most about, 1, 2, 3, 4, 5, 6, 7, 8, 9, 10, 11, 12, 13, 14, 15, 16, 17, 18, 19, 20, 25, 30, 35, 40, 45, 50, or a number or a range between any two of these values.

In some embodiments, the gas-flow control device further comprises a plurality of gas injection valves and a plurality of gas extraction valves disposed on and in the plate. The inlet open end of each of the plurality of gas injection microchannels can be connected to a gas injection valve of the plurality of gas injection valves. The outlet open end of each of the plurality of gas extraction microchannels can be connected to a gas extraction valve of the plurality of gas extraction valves.

The arrangement of the gas injection valves and gas extraction valves are not limited, and can be adjusted according to the surface of the plate or the application of the gas-flow control device. For example, the gas injection valves and gas extraction valves may be arrange apart from each other, or in proximity of each other, on the plate. The gas injection valves can be arranged into one or more groups, and the gas extraction valves can be arrange into one or more groups, where the valves are near each other within a group. Two groups may be near each other, or apart from each other, on the plate. All the gas injection valves and gas extraction valves may be arranged in a condensed manner, for example, in one area on the plate. In some embodiments, a majority (e.g., 60%, 70%, 80%, 90%, or more) of, or all of, the plurality of gas injection valves are arranged on one end of the plate. In some embodiments, a majority (e.g., 60%, 70%, 80%, 90%, or more) of, or all of, the plurality of gas extraction valves are arranged on one end of the plate. In some embodiments, a majority (e.g., 60%, 70%, 80%, 90%, or more) of, or all of, the plurality of gas injection valves and a majority of, or all of, the plurality of gas extraction valves are arranged on one end of the plate. The number of gas valves (e.g., gas injection valves or gas extraction valves) arranged on one end of the plate can be, for example, 5, 6, 7, 8, 9, 10, 11, 12, 13, 14, 15, 16, 17, 18, 19, 20, 25, 30, 35, 40, 45, 50, or more.

The number of the gas injection valves are not limited. For example, the number of the gas injection valves is, is about, is at least, is at least about, is at most, or is at most about 2, 3, 4, 5, 6, 7, 8, 9, or 10. In some embodiments, the gas-flow control device includes 6, 7, 8, 9, or 10 gas injection valves.

The number of the gas extraction valves are not limited. For example, the number of the gas extraction valves is, is about, is at least, is at least about, is at most, or is at most about 2, 3, 4, 5, 6, 7, 8, 9, or 10. In some embodiments, the gas-flow control device includes 2, 3, 4, or 5 gas extraction valves.

In some embodiments, the number of the gas injection valves is the same as the number of gas injection microchannels. In some embodiments, the number of the gas injection valves is greater than the number of gas injection microchannels. In some embodiments, the number of the gas extraction valves is the same as the number of gas extraction microchannels. In some embodiments, the number of the gas extraction valves is greater than the number of gas extraction microchannels. In some embodiments, one or more of the plurality of injection valves (e.g., 2, 3, 4, 5, 6, 7, 8, 9, 10, 15, 20, or more) is each connected to a gas injection microchannel of the plurality of gas injection microchannels (or channels). In some embodiments, one or more of the plurality of injection valves (e.g., 2, 3, 4, 5, 6, 7, 8, 9, 10, 15, 20, or more) is not connected to a gas injection microchannel of the plurality of gas injection microchannels (or channels). In some embodiments, one or more of the plurality of extraction valves (e.g., 2, 3, 4, 5, 6, 7, 8, 9, 10, 15, 20, or more) is each connected to a gas extraction microchannel of the plurality of gas extraction microchannels (or channels). In some embodiment, one or more of the plurality of extraction valves (e.g., 2, 3, 4, 5, 6, 7, 8, 9, 10, 15, 20, or more) is not connected to a gas extraction microchannel of the plurality of gas extraction microchannels (or channels).

In some embodiments, one, one or more, or each of the plurality of gas injection valves when pressurized with a driving gas and in an open state injects the driving gas in a direction from the inlet open end of a gas injection microchannel of the plurality of gas injection microchannels to the outlet open end of the gas injection microchannel. In some embodiments, one, one or more, or each of the plurality of gas extraction valves under suction and in an open state allows a gas to flow in a direction from the inlet open end of a gas extraction microchannel of the plurality of gas injection microchannels to the outlet open end of the gas extraction microchannel.

In some embodiments, a gas injection valve of the plurality of gas injection valves controls an amount of gas exiting the outlet open end of the corresponding gas injection microchannel. In some embodiment, a gas extraction valve of the plurality of gas extraction valves controls an amount of gas entering the inlet open end of the corresponding gas extraction microchannel.

In some embodiments, one, one or more, or each of the plurality of gas injection valves is a solenoid valve. In some embodiments, one, one or more, or each of the plurality of gas extraction valves is a solenoid valve. Suitable solenoid valve include those commercially available products, and can be selected according the design and application of the gas-flow control device.

In some embodiments, the plate further comprises an observation window. The observation window can be, for example, an opening or a hole on the plate. The observation window can be any suitable shape, including, but not limited to, a rectangle, a circle, an oval, an ellipse, a semicircle, a trapezoid, or a combination thereof.

The size of a gas injection microchannel, an inlet or outlet of a gas injection microchannel, a gas extraction microchannel, an inlet or outlet of a gas extraction microchannel, or the gas-flow control device can be determined, for example, by measuring a width, a length, a depth (or height), a radius, a radius, or a circumference of such microchannel, inlet, outlet, or device. In some embodiments, a length of a gas injection microchannel or a length of a gas injection microchannel is measured. In some embodiments, a diameter of an inlet or outlet of a gas injection microchannel or a diameter of an inlet or outlet of a gas extraction microchannel is measured.

In some embodiments, a size (e.g., width, length, depth (or height), radius, diameter, or circumference) of one, one or more, or each of the plurality gas injection microchannels is 1 mm to 20 cm. This includes a size (for example, as measured by length) that is, is about, is at least, is at least about, is at most, or is at most 1 mm, 2 mm, 3 mm, 4 mm, 5 mm, 6 mm, 7 mm, 8 mm, 9 mm, 1 cm, 2 cm, 3 cm, 4 cm, 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, or 20 cm, or a number or a range between any two of these values.

In some embodiments, a size (e.g., width, length, depth (or height), radius, diameter, or circumference) of the inlet and/or the outlet of one, one or more, or each of the plurality gas injection microchannels is 0.1 mm to 5 mm. This includes a size (for example, as measured by diameter) that is, is about, is at least, is at least about, is at most, or is at most 0.1 mm, 0.2 mm, 0.3 mm, 0.4 mm, 0.5 mm, 0.6 mm, 0.7 mm, 0.8 mm, 0.9 mm, 1 mm, 1.5 mm, 2 mm, 2.5 mm, 3 mm, 3.5 mm, 4 mm, 4.5 mm, or 5 mm, or a number or a range between any two of these values.

In some embodiments, a size (e.g., width, length, depth (or height), radius, diameter, or circumference) of one, one or more, or each of the plurality gas extraction microchannels is 1 mm to 20 cm. This includes a size (for example, as measured by length) that is, is about, is at least, is at least about, is at most, or is at most 1 mm, 2 mm, 3 mm, 4 mm, 5 mm, 6 mm, 7 mm, 8 mm, 9 mm, 1 cm, 2 cm, 3 cm, 4 cm, 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, or 20 cm, or a number or a range between any two of these values.

In some embodiments, a size (e.g., width, length, depth (or height), radius, diameter, or circumference) of the inlet and/or the outlet of one, one or more, or each of the plurality gas extraction microchannels is 0.1 mm to 5 mm. This includes a size (for example, as measured by diameter) that is, is about, is at least, is at least about, is at most, or is at most 0.1 mm, 0.2 mm, 0.3 mm, 0.4 mm, 0.5 mm, 0.6 mm, 0.7 mm, 0.8 mm, 0.9 mm, 1 mm, 1.5 mm, 2 mm, 2.5 mm, 3 mm, 3.5 mm, 4 mm, 4.5 mm, or 5 mm, or a number or a range between any two of these values.

In some embodiments, a size (e.g., width, length, depth (or height), or circumference) of the gas-flow control device is 5 mm to 40 cm. This includes a size (for example, as measured by length or diameter) that is, is about, is at least, is at least about, is at most, or is at most 5 mm, 6 mm, 7 mm, 8 mm, 9 mm, 1 cm, 2 cm, 3 cm, 4 cm, 5 cm, 6 cm, 7 cm, 8 cm, 9 cm, 10 cm, 15 cm, 20 cm, 25 cm, 30 cm, 35 cm, or 40 cm, or a number or a range between any two of these values.

A cross-sectional shape of the gas injection microchannels and the gas extraction microchannels can be any suitable shape. In some embodiments, a cross-sectional shape of one, one or more, or each of the plurality of gas injection microchannels is a circle, a rectangle, an ellipse, a semicircle, a trapezoid, or a combination thereof. In some embodiments, a cross-sectional shape of one, one or more, or each of the plurality of gas extraction microchannels is a circle, a rectangle, an ellipse, a semicircle, a trapezoid, or a combination thereof.

The plate may include one or more layers (or boards). For example, the plate may include a plurality of layers (e.g., 2, 3, 4, 5, 6, 7, 8, 9, 10 or more), and one layer may be attached, bonded, coupled, or laminated to at least one other layer of the plurality of layers. In some embodiments, the plate comprises a plurality of layers, and each of the plurality of layers is reversibly coupled to at least one other layer of the plurality of layers.

In some embodiments, one or more gas injection valves of the plurality of injection valves and/or one or more gas extraction valves of the plurality extraction valves are disposed on and through a first layer of the plurality of layers. For example, the injection valve or the extraction valve is disposed through a first layer, and is in contact with (but not disposed through) another layer (such as a cover layer) of the plurality of layers. For example, the injection valve or the extraction valve is disposed through all the layers of the of the plurality of layers of the plate.

The microchannels can be formed, for example, by the plurality of layers of the plate. In some embodiments, the first layer comprises a plurality of grooves, and a second layer of the plurality of layers cover the plurality of grooves to form the plurality of gas injection microchannels and/or the plurality of gas extraction microchannels. In some embodiments, both the first layer and the second layer comprise grooves, and the gas injection microchannels and/or the gas extraction microchannels are formed by the first layer and the second layer. The grooves of the first layer may match the grooves of the second layer to form a complete microchannel.

In some embodiments, the one or more gas injection valves and the one or more gas extraction valves are disposed in or through a second layer of the plurality of layers. Optionally, one or more gas injection microchannels of the plurality of gas injection microchannels and/or one or more gas extraction microchannels of the plurality of gas extraction microchannels can be formed between and/or by the first layer and the second layer. Optionally, the second layer can be a cover layer. In some embodiments, the plurality of layers comprises a third layer that is a cover layer.

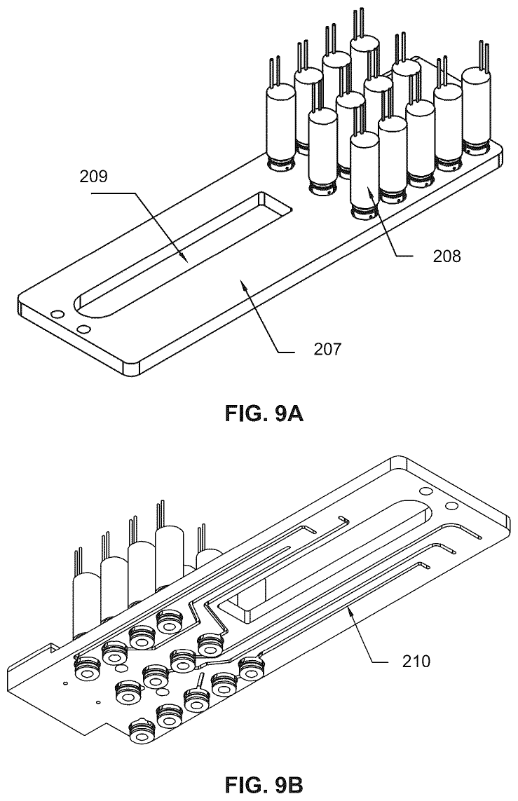

A non-limiting illustration of the present gas-flow control device is provided in FIGS. 9A-9B. The device is also referred to as a gas path control plate 207. At least two mutually independent driving gas path channels 210 (FIG. 9B, also referred to as a gas injection microchannel or a gas extraction microchannel herein) are provided inside the integrated gas path control plate 207.

The integrated gas path control plate 207 is provided with grooves therein to form (or integrate) the driving gas path channels 210, and the driving gas path channels 210 are independent of each other and do not communicate with each other. A driving gas can be used as a driving force for injecting reaction reagents to transport the reaction reagents in a flow path. A gas can be extracted from the driving gas path channels 210 to extract a waste or product.

It should be noted that in this specific embodiment, the formation manner of the driving gas path channels 210 can adopts any one of the following two solutions, or a combination thereof:
Solution 1: the integrated gas path control plate 207 is an integral structure, and the driving gas path channels 210 are directly formed in the integrated gas path control plate 207 by means of casting, drilling, additive manufacturing, or the like;
Solution 2: the integrated gas path control plate 207 is a split structure, which is formed by laminating an upper gas path plate and a lower gas path plate, the laminating surface between the upper gas path plate and/or lower gas path plate is provided with gas path grooves, and after the upper gas path plate and the lower gas path plate are attached to each other, the gas path grooves are closed to form the driving gas path channels 210; it can certainly be understood that in this solution, the gas path grooves may be provided on a lower surface of the upper gas path plate or an upper surface of the lower gas path plate, or may be provided on both the lower surface of the upper gas path plate and the upper surface of the lower gas path plate; as shown in FIG. 9B, the gas path grooves are provided on the lower surface of the upper gas path plate; the lower gas path plate is not shown in the figure, but it can also be understood that when the lower gas path plate and the upper gas path plate are attached to each other, the gas path grooves shown in FIG. 9B are closed by the lower gas path plate to form the driving gas path channels 210.

Inlet ends of the driving gas path channels 210 are provided with solenoid valves 208. A gas extraction end of the waste gas extraction channel is provided with a solenoid valve 208, and the solenoid valve 208 is used to control the gas extraction amount. A gas extraction end of the product gas extraction channel is provided with a solenoid valve 208. The solenoid valves 208 include one or more gas injection valves, one or more gas extraction valves, or both. The solenoid valves 208 are used to control the feeding amount of the driving gas or the amount of the extracted gas. The solenoid valves 208 are arranged in a concentrated manner on a surface of the integrated gas path control plate 207.

Different types of solenoid valves 208 are provided to achieve control of the entire flow path. Different solenoid valves 208 implement different control functions (e.g., injection and extraction). Mounting positions are reserved for the solenoid valves 208 on the surface of the integrated gas path control plate 207, so as to facilitate mounting and dismounting of the solenoid valves 208. In addition, the solenoid valves 208 are integrally mounted on the surface of the integrated gas path control plate 207, so that a fluid can pass through the flow path inside the integrated gas path control plate 207 to achieve control of the entire flow path.

The integrated gas path control plate 207 is further provided with an observation window 209.

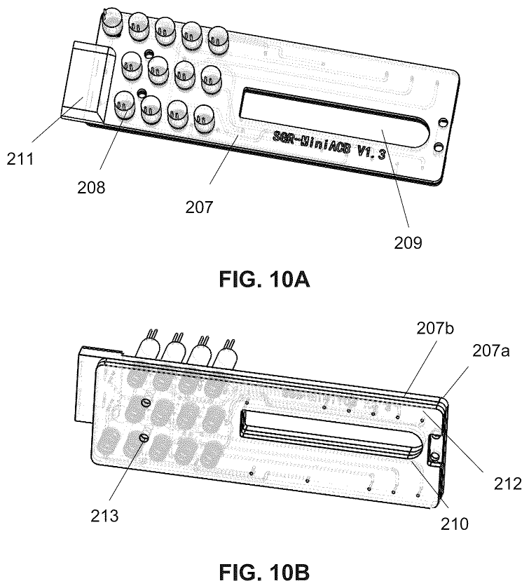

FIGS. 10A and 10B show a representative gas path control plate 207 with laminated upper gas path plate 207a and lower gas path plate 207b. The driving gas path channels 210 may be formed by gas path grooves on the laminating surface of the upper gas path plate and/or the lower gas path plate. The gas path control plate can also include additional structures. For example, the gas path control plate 207 can include a structure 211 to which a drive module can be attached.

### Reaction Module

Disclosed herein include embodiments of a reaction module. In some embodiments, a reaction module comprises a microfluidic device as described herein. The reaction module can comprise a gas-flow control device as described herein capable of detachably coupling to and/or forming an air-tight seal with the microfluidic device.

In some embodiments, a reaction module comprises a microfluidic device described herein. The reaction module can comprise a gas-flow control device described herein. An area on a surface (e.g., a lower or bottom surface) of the gas-flow control device surrounding the outlet open end of one (or one or more, or each) gas injection microchannel of plurality of gas injection microchannels can be capable of detachably coupling to and/or forming a seal, such as an air tight seal, with one reagent reservoir (or a corresponding reagent reservoir) of the plurality of reagent reservoirs. An area on the surface of the gas-flow control device surrounding the inlet open end of the waste gas extraction microchannel is capable of detachably coupling to and/or forming a seal, such as an air tight seal, with the waste reservoir to result. An area on the surface of the gas-flow control device surrounding the inlet open end of the product gas extraction microchannel can be capable of detachably coupling to and/or forming a seal, such as an air tight seal, with the product reservoir. In some embodiments, an outlet of a gas injection valve (or an inlet of a gas extraction valve) is open into the area not through a gas injection microchannel (or a gas extraction microchannel).

In some embodiments, a reaction module comprises a microfluidic device described herein. The reaction module can comprise a gas-flow control device described herein. The gas-flow control device can be capable of detachably coupling to and/or forming a seal, such as an air tight seal, with one reagent reservoir (or one or more, or each) of the plurality of reagent reservoirs to result in a space comprising the outlet open end of one gas injection microchannel (or a corresponding gas injection microchannel) of plurality of gas injection microchannels. The gas-flow control device can be capable of detachably coupling to and/or forming a seal, such as an air tight seal, with the waste reservoir to result in a space comprising the inlet open end of the waste gas extraction microchannel. The gas-flow control device can be capable of detachably coupling to and/or forming a seal, such as an air tight seal, with the product reservoir to result in a space comprising the inlet open end of the product gas extraction microchannel In some embodiments, an outlet of a gas injection valve (or an inlet of a gas extraction valve) is open into the space not through a gas injection microchannel (or a gas extraction microchannel).

In some embodiments, the gas-flow control device is attached to and/or forms a seal, such as an air tight seal with the microfluidic device. The gas-flow control device can be attached to and/or form a seal, such as an air tight seal, via a silicone pad sandwiched between the gas-flow control device and the microfluidic device. The silicon pad can comprise a plurality of through holes. The through holes can allow gaseous communication of the outlet opening ends of the gas injection microchannels with reservoirs, such as reagent reservoirs. The through holes can allow gaseous communication of the inlet opening end of a gas extraction microchannel with the waste reservoir. The through holes can allow gaseous communication of the inlet opening end of a gas extraction microchannel with the product reservoir. When the silicon pad is aligned with and sandwiched between the gas-flow control device and the microfluidic device, a plurality of through holes at positions corresponding to the positions of the outlet opening ends of the gas injection microchannels and the inlet opening ends of the gas extraction microchannels.

In some embodiments, one, one or more, or each of the plurality of gas injection microchannels is in gaseous communication with one of the plurality of reagent. The outlet open end of one, one or more, or each of the plurality of gas injection microchannels can be open to one of the plurality of reagent reservoirs. In some embodiments, the waste gas extraction microchannel is in gaseous communication with the waste reservoir. The inlet open end of the waste gas extraction microchannel can be open to the waste reservoir. In some embodiments, the product gas extraction microchannel is in gaseous communication with the product reservoir. The inlet open end of the product gas extraction microchannel can be open to the product reservoir. In some embodiments, the reagent exchange gas injection microchannel is in gaseous communication with the reagent exchange reservoir. The outlet open end of the reagent exchange gas injection microchannel can be open to the reagent exchange reservoir. In some embodiments, the reagent exchange gas extraction microchannel is in gaseous communication with the reagent exchange reservoir. The inlet open end of the reagent exchange gas extraction microchannel can be open to the reagent exchange reservoir.

In some embodiments, when a driving gas exits the outlet of the gas injection microchannel into the reagent reservoir, a reagent in the reagent reservoir is driven from the reagent reservoir through a fluid microchannel of the plurality of fluid microchannels into the reagent exchange reservoir. When a gas exits the inlet of the waste gas extraction microchannel, one or more reagents in the reagent exchange reservoir can be pulled from the reagent exchange reservoir into the reaction chamber then into the waste reservoir. When a gas exits the inlet of the product gas extraction microchannel, one or more reagents in the reagent exchange reservoir can be pulled from the reagent exchange reservoir into the reaction chamber then into the product reservoir

In some embodiments, when a driving gas exits (e.g., pushed into) the outlet of the gas injection microchannel into the reagent reservoir, (i) a reagent in the reagent reservoir is driven (e.g., pushed) from the reagent reservoir through a fluid microchannel of the plurality of fluid microchannels into the reagent exchange reservoir, and (ii) a gas in the reagent exchange reservoir exits the reagent exchange reservoir. When a driving gas exits (e.g., pushed into) the outlet of the reagent exchange gas injection microchannel into the reagent exchange reservoir, one or more reagents in the reagent exchange reservoir can be driven from the reagent exchange reservoir into the reaction chamber. Alternatively or additionally, when a gas exits the inlet of the waste gas extraction microchannel, one or more reagents in the reagent exchange reservoir can be pulled from the reagent exchange reservoir into the reaction chamber then into the waste reservoir. Alternatively or additionally, when a gas exits the inlet of the product gas extraction microchannel, one or more reagents in the reagent exchange reservoir can be pulled from the reagent exchange reservoir into the reaction chamber then into the product reservoir. Optionally, the one or more reagents in the reagent exchange reservoir can be mixed in the reagent exchange reservoir. For example, two or more reagents in two reagent reservoirs can be injected into the reagent exchange reservoir by the driving gas exiting the outlets of the corresponding gas injection microchannels. The two or more reagents can be injected into the reagent exchange reservoir without being injected into the reaction chamber, which allows the two or more reagents to mix in the reagent exchange reservoir to generate a mixture. The mixture in the reagent exchange reservoir can be injected into the reaction chamber, for example, by a driving gas exiting the outlet of the reagent exchange gas injection microchannel.

In some embodiments, when a gas exits the inlet of the waste gas extraction microchannel from the waste reservoir, a waste in the reaction chamber is pulled from the reaction chamber through the waste fluid microchannel into the waste reservoir. In some embodiments, when a gas exits the inlet of the product gas extraction microchannel from the product reservoir, a product in the reaction chamber is pulled from the reaction chamber through the product fluid microchannel into the product reservoir. Optionally, the product can be generated using at least one reagent.

In some embodiments, when the gas injection microchannel is under a positive pressure and/or the gas injection valve is in an open state and/or (ii) the waste gas extraction microchannel is under a negative pressure and/or the waste gas extraction valve is in an open state, (1) a reagent in the reagent reservoir is driven (e.g., pushed) through a fluid microchannel of the plurality of fluid microchannels into the reagent exchange reservoir then into the reaction chamber, and/or (2) a waste generated in the reaction chamber from the reagent is driven (e.g., sucked or pulled) from the reaction chamber through the waste fluid microchannel into the waste reservoir.

In some embodiments, when the gas injection microchannel is under a positive pressure and/or the gas injection valve is in an open state and/or when the product gas extraction microchannel is under a negative pressure and/or the product gas extraction valve is in an open state, a reagent in the reagent reservoir is driven (e.g., pushed) through a fluid microchannel of the plurality of fluid microchannels into the reagent exchange reservoir then into the reaction chamber, and a product generated in the reaction chamber from the reagent is driven (e.g., pulled or sucked) from the reaction chamber through the product fluid microchannel into the product reservoir.

In some embodiments, when the gas injection microchannel is under a positive pressure and/or the gas injection valve is in an open state, a reagent in the reagent reservoir is driven (e.g., pushed) through a fluid microchannel of the plurality of fluid microchannels into the reagent exchange reservoir. When the waste gas extraction microchannel is under a negative pressure and/or the waste gas extraction valve is in an open state, (1) a reagent in the reagent exchange reservoir can be pulled (or sucked) into the reaction chamber, and/or (2) a waste generated in the reaction chamber from the reagent is pulled from the reaction chamber through the waste fluid microchannel into the waste reservoir. When the product gas extraction microchannel is under a negative pressure and/or the product gas extraction valve is in an open state, (1) a reagent in the reagent exchange reservoir can be pulled into the reaction chamber, and/or (2) a product generated in the reaction chamber from the reagent can be pulled (or sucked) from the reaction chamber through the product fluid microchannel into the product reservoir, optionally wherein the product is generated using the reagent.

In some embodiments, the mixing gas extraction microchannel is under a negative pressure and/or the mixing gas extraction valve is in an open state, two or more reagents in the reagent exchange reservoir can be pulled (or sucked) from the reagent exchange reservoir into the mixing reservoir, thereby mixing the two or more reagents. When the mixing gas injection microchannel is under a positive pressure and/or the mixing gas injection valve is in an open state, the one or more reagents in the mixing reservoir can be driven (e.g., pushed) into the reagent exchange reservoir. When the waste gas extraction microchannel is under a negative pressure and/or the waste gas extraction valve is in an open state, the one or more reagents in the reagent exchange reservoir can be pulled from the reagent exchange reservoir into the reaction chamber where a waste is generated in the reaction chamber from the one or more reagents and the waste is pulled from the reaction chamber through the waste fluid microchannel into the waste reservoir. When the product gas extraction microchannel is under a negative pressure and/or the product gas extraction valve is in an open state, the one or more reagents in the reagent exchange reservoir can be pulled from the reagent exchange reservoir into the reaction chamber where a product is generated using the one or more reagents and the product is pulled into the product reservoir.

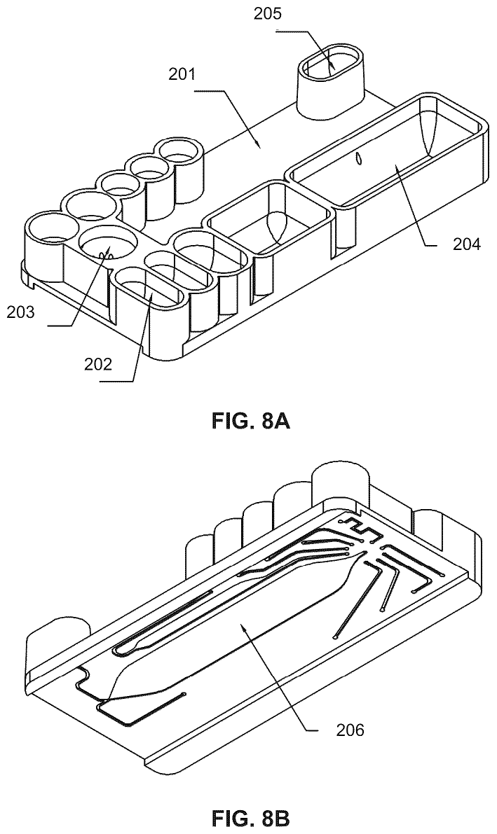

A non-limiting illustration of the present reaction module is provided in FIGS. 8A-8B and 9A-9B. The illustrated reaction module includes an integrated gas path control plate 207 (FIG. 9A, also referred to as a gas-flow control device herein) and a reaction plate 201 (FIG. 8A, also referred to as a microfluidic device herein) attached to each other. At least two mutually independent driving gas path channels 210 are provided inside the integrated gas path control plate 207. A surface of the cell reaction plate 201 on a side thereof attached to the integrated gas path control plate 207 is provided with reagent reservoirs 202 of the same number as the driving gas path channels 210. Each driving gas path channel 210 independently communicates with one reagent reservoir 202. A surface of the reaction plate 201 on a side thereof away from the integrated gas path control plate 207 is provided with a reaction chamber 206 (FIG. 8B). The reaction chamber 206 communicates with the reagent reservoirs 202. Reaction reagents are injected into the reagent reservoirs 202 in advance, a driving gas is injected into the reagent reservoirs 202 through the driving gas path channels 210, and the reaction reagents in the reagent reservoirs 202 flow into the reaction chamber 206 under the pressure of the driving gas.

During use, the driving gas is injected into different driving gas path channels 210, so that reaction reagents in different reagent reservoirs 202 can be injected into the reaction chamber 206 one by one. The injection processes of different reaction reagents are independent of each other and do not affect each other, so that the flow path control process of the reaction reagents is stable and reliable, and the entire cell reaction module can be mounted easily and occupies a small space.

It should be noted that the reaction plate 201 is also provided with microchannels (also referred to as fluid microchannels herein) therein for reaction reagents to flow therethrough. Reference may be made to the above description of the driving gas path channels 210 for the formation manner of the microchannels. That is, the reaction plate 201 may be an integral structure or a split structure. When the reaction plate 201 is an integral structure, the microchannels are directly formed in the reaction plate 201 by means of casting, drilling, additive manufacturing, or the like. Certainly, the reaction plate 201 may be a split structure formed by laminating an upper reaction plate and a lower reaction plate. The laminating surface of the upper reaction plate and/or lower reaction plate is provided with flow path grooves, and after the upper reaction plate and the lower reaction plate are attached to each other, the flow path grooves are closed to form microchannels. As shown in FIG. 8B, the flow path grooves are provided on a lower surface of the upper reaction plate. The lower reaction plate is not shown in the figure, but it can also be understood that after the lower reaction plate and the upper reaction plate are attached to each other, the flow path grooves shown in FIG. 8B are closed by the lower reaction plate to form the microchannels.

The surface of the reaction plate on the side thereof 201 attached to the integrated gas path control plate 207 is further provided with a buffer reservoir 203 (also referred to as a reagent exchange reservoir herein). The reagent reservoirs 202 and the reaction chamber 206 each independently communicate with the buffer reservoir 203. The driving gas path channels 210, the reagent reservoirs 202, and the buffer reservoir 203 communicate with each other in sequence in a flow direction of the driving gas. The driving gas is fed into the reagent reservoirs 202 through the driving gas path channels 210, and the reaction reagents stored in the reagent reservoirs 202 are driven into the buffer reservoir 203 one by one, and are injected into the reaction chamber 206 through the buffer reservoir 203. During the cell reaction, the reaction reagents stored in the reagent reservoirs 202 are pressed into the buffer reservoir 203 under the pressure of the driving gas, and the flow of the driving gas is adjusted by the solenoid valves 208, so as to change the injection amount of the reaction reagents entering the buffer reservoir 203. Optionally, a control module is integrally provided in the integrated gas path control plate 207 provided in the present cell reaction module, and the control module is electrically connected to the solenoid valves 208, so as to realize automatic control on the flow of the driving gas.

The surface of the reaction plate on the side thereof 201 attached to the integrated gas path control plate 207 is further provided with a waste reservoir 204, and the waste reservoir 204 communicates with the reaction chamber 206. A waste gas extraction channel (or microchannel) is provided inside the integrated gas path control plate 207, and the waste gas extraction channel communicates with the waste reservoir 204. The reaction chamber 206, the waste reservoir 204, and the waste gas extraction channel communicate with each other in sequence in a gas extraction direction. A waste liquid after the reaction ends in the reaction chamber 206 is drawn into the waste reservoir 204 by means of gas extraction in the waste gas extraction channel using a solenoid valve 208.

It should be noted that the waste gas extraction channel participates in two process steps, specifically:

First, during the cell reaction, after the reaction reagents are injected into the buffer reservoir 203 from the reagent reservoirs 202, the waste gas extraction channel is used to perform gas extraction. Since the waste reservoir 204, the reaction chamber 206, and the buffer reservoir 203 communicate with each other in sequence, the reaction reagents temporarily stored in the buffer reservoir 203 is drawn into the reaction chamber 206 under the action of the suction. It should be particularly noted that the negative pressure for gas extraction cannot be excessively large to prevent the reaction reagents entering the reaction chamber 206 from being further drawn into the waste reservoir 204.

Second, after the cell reaction ends, the waste gas extraction channel is used again for gas extraction, and the reaction waste liquid remaining in the reaction chamber 206 is drawn into the waste reservoir 204 under the action of the suction.

The surface of the reaction plate on the side thereof 201 attached to the integrated gas path control plate 207 is further provided with a product reservoir 205. The product reservoir 205 communicates with the reaction chamber 206. A product gas extraction channel (or microchannel) is provided inside the integrated gas path control plate 207, and the product gas extraction channel communicates with the product reservoir 205. The reaction chamber 206, the product reservoir 205, and the product gas extraction channel communicate with each other in sequence in a gas extraction direction. A reaction product obtained in the reaction chamber 206 is drawn into the product reservoir 205 by means of gas extraction in the product gas extraction channel using a solenoid valve 208. After the cell reaction ends, the product gas extraction channel is used to perform gas extraction on the product reservoir 205, so that the reaction product in the reaction chamber 206 enters the product reservoir 205. In order to draw the reaction product in the reaction chamber 206 completely into the product reservoir 205, the gas extraction amount needs to be strictly controlled. The solenoid valve 208 may be controlled by a control module to automatically control the gas extraction amount.

A silicone pad 212 (FIG. 10B) is sandwiched between the integrated gas path control plate 207 and the cell reaction plate 201. A through hole 213 is provided in the silicone pad, and the integrated gas path control plate 207 and the cell reaction plate 201 are connected by means of the through hole. The solenoid valves 208 are arranged in a concentrated manner on a surface of the integrated gas path control plate on a side thereof 207 away from the cell reaction plate 201. The reaction condition within the reaction chamber 206 is observed through the observation window 209.

The present disclosure also provides a method of using the cell reaction module provided in the aforementioned specific embodiment to perform a cell reaction, the method including:
(1) feeding a driving gas into the driving gas path channels 210 through the solenoid valves 208, injecting the driving gas into corresponding reagent reservoirs 202 along the independent driving gas path channels 210, and pressing the reaction reagents stored in the reagent reservoirs 202 into the buffer reservoir 203 under the pressure of the driving gas;
(2) performing gas extraction on the waste liquid reservoir 204 through the waste liquid gas extraction channel, so that the reaction reagents in the buffer reservoir 203 are drawn into the reaction chamber to complete reagent injection;
(3) repeatedly performing step (1) and step (2), so that the reaction reagents in the reagent reservoirs 202 are all injected into the reaction chamber 206 to perform a cell reaction; and
(4) after the cell reaction ends, performing gas extraction on the waste liquid reservoir 204 through the waste liquid gas extraction channel again, so that a waste liquid in the reaction chamber 206 enters the waste liquid reservoir 204; performing gas extraction on the product reservoir 205 through the product gas extraction channel, so that a reaction product in the reaction chamber 206 enters the product reservoir 205.

### Sample Preparation Device

Disclosed herein include embodiments of a sample preparation device. In some embodiments, a sample preparation device comprises a reaction module as described herein. The sample preparation device can comprise a heating element in contact with the microfluidic device of the reaction module. In some embodiments, the microfluidic device is sandwiched between the gas-flow control device and the heating element.

In some embodiments, a sample preparation device comprises a gas-flow control device as described herein capable of detachably coupling to and/or forming an air-tight seal with a microfluidic device as described herein. The sample preparation device can comprise a heating element for heating the microfluidic device. In some embodiments, the microfluidic device is sandwiched between the gas-flow control device and the heating element when the microfluidic device, the gas-flow control device, and the heating element are in an assembled state. Optionally, the microfluidic device is below the gas-flow control device in the assembled state. Optionally, the heating element is below the microfluidic device in the assembled state

In some embodiments, the sample preparation device further comprises an injection pump for providing gas to the plurality of gas injection valves and/or a extraction pump for providing suction to the gas extracting valves. For example, the injection pump can inject gas into the gas-flow control device through a gas injection valves, thereby providing a positive pressure at an outlet of an gas injection microchannel. For example, the extraction pump can extract gas from the gas-flow control device through a gas extract valves, thereby providing a negative pressure (or suction) at an inlet of an gas extraction microchannel. Optionally, the injection pump is the extraction pump. For example, a pump can have a dual function of gas injection and gas extraction. Any suitable location or arrangement of the injection pump and/or the extraction pump relative to the reaction module may be implemented herein. Optionally, the injection pump and/or the extraction pump is adjacent the reaction module and/or below the reaction module when the sample preparation device is in an upright orientation. In some embodiments, the injection pump and the extraction pump are in a form of a pump assembly.

In some embodiments, the sample preparation device further comprises a control unit in electrical communication with and/or controls the plurality of gas injection valves, the plurality of gas extraction valves, the heating element, the injection pump, and/or the extraction pump. Any suitable location or arrangement of the control unit relative to the reaction module, the heating element, the injection pump, and/or the extraction pump may be implemented herein. Optionally, the control unit can be adjacent the reaction module and/or below the reaction module when the sample preparation device is in an upright orientation. Optionally, the control unit can be adjacent the injection pump and/or the extraction pump.

In some embodiments, the sample preparation device further comprises a housing to which the gas-flow control device, the heating element, the control unit, the injection pump, and/or the extraction pump are attached. The housing can include, for example, at least one frame, at least shelf, at least one compartment, at least one platform, or a combination thereof.

The sample preparation device can have a size as measured by, for example, a width, a length, a height, a diameter, or a circumference. In some embodiments, the size is measured by a width or a height of the sample preparation device. In some embodiments, a size of the sample preparation device is 10 mm to 100 cm. For example, the size of the sample preparation device is, is about, is at least, is at least about, is at most, or is at most 10 mm, 20 mm, 30 mm, 40 mm, 50 mm, 60 mm, 70 mm, 80 mm, 90 mm, 10 cm, 15 cm, 20 cm, 25 cm, 30 cm, 35 cm, 40 cm, 45 cm, 50 cm, 55 cm, 60 cm, 65 cm, 70 cm, 75 cm, 80 cm, 85 cm, 90 cm, 95 cm, or 100 cm, or a number or a range between any two of these values.

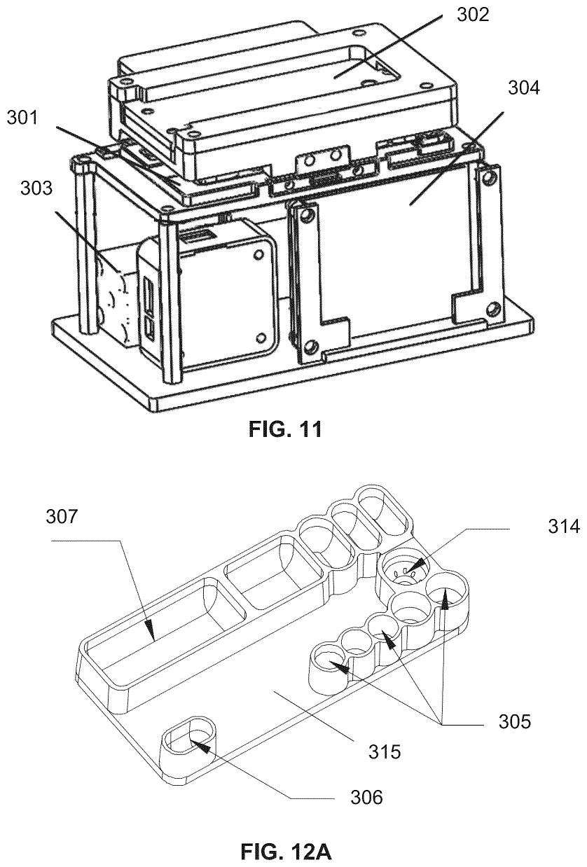

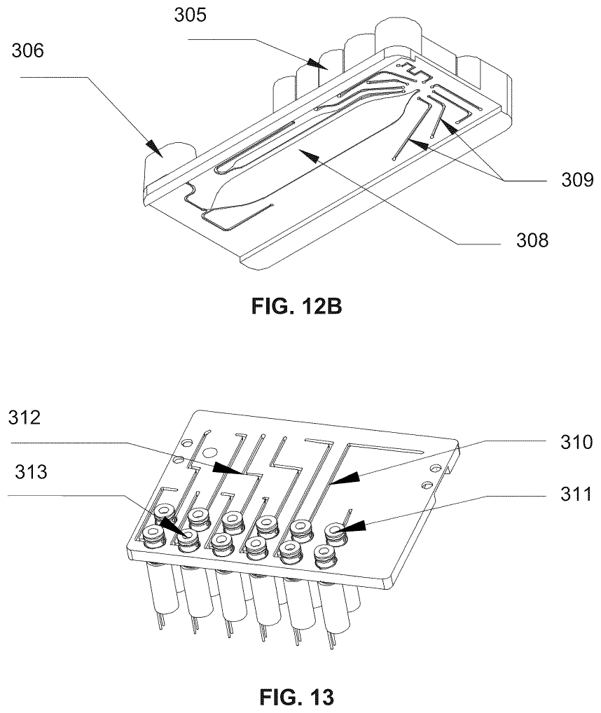

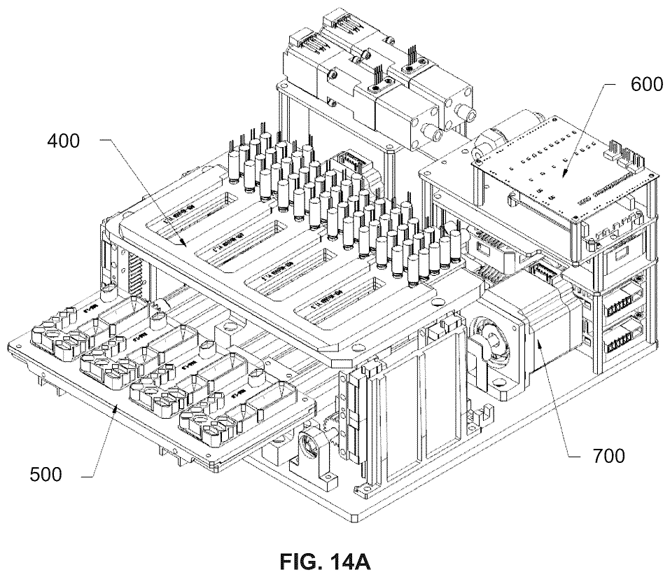

A non-limiting illustration of the present sample preparation device (such as a cell sample preparation device) is provided in FIGS. 11, 12A-12B, and 13. The preparation device includes a frame (also referred to as a housing herein). The frame is provided with a reaction module thereon. The reaction module includes a gas-path control board 302 (also referred to as a gas-flow control device herein) and a heating plate 301, and a reaction chip (also referred to as a microfluidic device herein) is sandwiched between the gas-path control board 302 and the heating plate 301. The gas-path control board 302 is provided with at least two mutually independent driving gas-path channels therein 312. The reaction chip includes a platform (also referred to as a reaction unit or a working unit herein) and a cell reaction plate 315 (also referred to as a reagent exchange unit herein) attached to each other. A surface of the cell reaction plate on a side thereof attached to the gas-path control board 302 is provided with reagent reservoirs 305 of the same number as the driving gas-path channels 312. Each driving gas-path channel 312 independently communicates with one reagent reservoir 305. A surface of the cell reaction plate on a side thereof attached to the platform is provided with a reaction chamber 308, and the reagent reservoirs 305 are all independently connected to the reaction chamber 308. A reaction reagent is injected into the reagent reservoir 305 in advance, and driving gas is injected into the reagent reservoir 305 via the driving gas-path channel 312 so as to press the reaction reagent in the reagent reservoir 305 into the reaction chamber 308.

In the present sample preparation device, via the arrangement of the reagent reservoirs 305 on the reaction chip, in combination with injection of gas into the reagent reservoirs 305 via the driving gas-path channels 312 on the gas-path control board 302, the injection of the reaction reagents into the reaction chamber 308 to perform cell reaction is thereby achieved. The reaction reagents are injected into a buffer reservoir 314 (also referred to as a reagent exchange reservoir herein) under the control of the driving gas, to achieve the injection of different reagents in batches or together, thereby achieving the quantitative injection of different reaction reagents, which effectively reduces the operation difficulty for an operator. The matching of structures of the gas-path control board 302 and the reaction chip simplifies the structure of the reaction unit. Further, via the arrangement of the heating plate 301 to heat the reaction chip, the present sample preparation device is capable of performing reverse transcription, and the present sample preparation device has benefits such as a simple structure, easy operation, a small footprint and high adaptability.

Further, the cell reaction plate is further provided with a product reservoir 306 and a waste reservoir 307 located on a surface thereof on the side of the reagent reservoir 305. The gas-path control board 302 is further provided with two gas extraction channels 310 therein. The product reservoir 306 and the waste reservoir 307 are each independently connected to one of the two gas extraction channels 310. The product reservoir 306 and the waste reservoir 307 are independently connected to the reaction chamber 308. The product reservoir 306 and the waste reservoir 307 are each connected to a gas extraction solenoid valve 311 via a gas extraction channel 310.

Via the arrangement of the product reservoir 306 and the waste reservoir 307, which respectively communicate with the gas extraction solenoid valves 311, the cell sample in the reaction chamber 308 can be drawn. In addition, by means of gas extraction performed on the waste reservoir 307, the reaction reagent in the buffer reservoir 314 is driven into the reaction chamber 308. After the reaction, the cell sample from the reaction is collected to the product reservoir 306 by subjecting the product reservoir 306 to gas extraction.

Further, the reagent reservoirs 305 are all connected to a plurality of gas injection solenoid valves 313 via the driving gas-path channels 312. The gas extraction solenoid valves 311 and the plurality of gas injection solenoid valves 313 are concentratedly arranged on a surface of the gas-path control board on the same side thereof 302. The gas extraction solenoid valves 311 and the plurality of gas injection solenoid valves 313 are concentratedly arranged on a surface of the gas-path control board on the same side thereof 302, thereby improving the integration degree of the device, avoiding the problem of messy pipelines, and reducing a footprint.

Further, the surface of the cell reaction plate on the side thereof on which the reagent reservoir 305 is located is provided with a buffer reservoir 314. The reagent reservoir 305 and the reaction chamber 308 independently communicate with the buffer reservoir. In a flow direction of the reaction reagents, the reagent reservoir 305, the buffer reservoir 314 and the reaction chamber 308 are connected in sequence.

Further, a silicone pad is arranged between the cell reaction plate 315 and the gas-path control board 302, and the silicone pad is provided with a hole thereon corresponding to an outlet position of the driving gas-path channels 312 on the gas-path control board 302.

Further, the gas-path control board 302 includes an upper gas-path board and a lower gas-path board laminated to each other. At least one gas-path groove is arranged on an attaching surface between the lower gas-path board and the upper gas-path board; the upper gas-path board is attached to the lower gas-path board, so that the gas-path groove is closed and sealed to form the driving gas-path channels 312 and the gas extraction channels 310.

Further, the surface of the cell reaction plate on the side thereof attached to the platform is further provided with a plurality of independent reagent flow grooves 309, and after the platform is attached to the cell reaction plate and sealed, the reagent flow grooves forms a plurality of reagent flow channels. The reagent reservoirs 305 are independently connected to the buffer reservoir 314 via the plurality of reagent flow channels. The buffer reservoir 314 is independently connected to the reaction chamber 308 via a reagent flow channel. Both the product reservoir 306 and the waste liquid reservoir 307 are independently connected to the reaction chamber 308 via independent reagent flow channels.

Further, the reaction unit is provided with a control unit 304 at the bottom thereof, and the control unit 304 is independently electrically connected to the heating plate 301, the plurality of gas injection solenoid valves 313 and the one or more gas extraction solenoid valves 311, and independently controls activation of the heating plate 301, activation of the plurality of gas injection solenoid valves 313, and activation of the gas extraction solenoid valves 311.

Further, the plurality of gas injection solenoid valves 313 and the gas extraction solenoid valves 311 are connected to a gas pump assembly 303 configured to control gas pressure in the plurality of gas injection solenoid valves 313 and the gas extraction solenoid valves 311. For example, the gas pump assembly 303 can be configured to provide a positive gas pressure in the reagent flow channels by the plurality of gas injection solenoid valves 313, and/or a negative gas pressure (suction) in the gas extraction channels by the gas extraction solenoid valves 311. The gas pump assembly 303 is located below the reaction module and is arranged side by side with the control unit 304. In the present sample preparation device, by integrally arranging the control unit 304 and the gas pump assembly 303 at the bottom of the reaction module, the footprint of the device is further reduced and the integration degree of the device is improved.

The present disclosure further provides a preparation method for preparing a cell sample by using the above sample preparation device, the preparation method particularly including the following steps:
(I) after injecting reaction reagents into the reagent reservoirs, placing the reaction chip between the gas-path control board 302 and the heating plate 301, activating, under the control of the control unit 304, a gas injection solenoid valve 313 and pressing a reaction reagent into the buffer reservoir 314 by injecting gas into the reagent reservoir connected to the gas injection solenoid valve, and activating, under the control of the control unit 304, the gas extraction solenoid valve 311 connected to the waste reservoir 307, and extracting gas from the waste reservoir 307 to draw the reaction reagent in the buffer reservoir 314 into the reaction chamber 308 to perform cell reaction;
(II) repeating the operation of Step (I) at least once, and controlling the heating plate 301 using the control unit 304 to heat the reaction chamber 308 to perform reverse transcription, and obtaining the cell sample; and
(III) activating, under the control of the control unit 304, the gas extraction solenoid valve 311 connected to the product reservoir 306, and drawing the cell sample in the reaction chamber 308 into the product reservoir 306.

### Sample Preparation Systems

Disclosed herein include embodiments of a reaction system (also referred to herein as a sample preparation system). In some embodiments, a reaction system comprises at least one gas-flow control device (e.g., 1, 2, 3, 4, 5, 6, 7, 8, 9, 10, 11, 12, 13, 14, 15, 16, 17, 18, 19, 20 or more) as described herein. The reaction system can comprise at least one drive module capable of detachably coupling to a microfluidic device as described herein to the gas-flow control device.

In some embodiments, the drive module comprises a microfluidic device drive module for moving the microfluidic device. Optionally, the microfluidic device drive module is for moving the microfluidic device horizontally between an away horizontal position and a coupling horizontal position. Optionally, when the microfluidic device drive module is in the away horizontal position, the microfluidic device is not below the gas-flow control device. For example, the away horizontal position can be a loading position for loading reagents into the microfluidic device. Optionally, when the microfluidic device drive module is in the coupling horizontal position, the microfluidic device is below the gas-flow control device or is detachably coupled to and/or forms an air-tight seal with the gas-flow control device. For example, the coupling horizontal position can be a coupling position (or contact position) that allows for the coupling or contact between the microfluidic device and the gas-flow control device. Optionally, the microfluidic device drive module can comprise at least one sliding table assembly. Optionally, the sliding table assembly comprises a sliding table, a sliding table support base, and a stepping motor.

The drive module can further comprise a gas-flow control drive module for moving the gas-flow control module. Optionally, the gas-flow control drive module is for moving the gas-flow control module vertically between an away vertical position and a contact vertical position. Optionally, when the microfluidic device drive module is in the coupling horizontal position and the gas-flow control drive module is in the away vertical position, the microfluidic device is below the gas-flow control device. Optionally, when the microfluidic device drive module is in the coupling horizontal position and the gas-flow control drive module is in the contact vertical position, the microfluidic device is detachably coupled to and/or forms an air-tight seal with the microfluidic device. Optionally, the gas-flow control drive module can comprise at least one push-rod assembly. Optionally, the push-rod assembly comprises a drive motor, a gear shaft attached to the drive motor, a slide rail, and a gear rack.

In some embodiments, the reaction system further comprises a heating element for heating the microfluidic device. Optionally, the heating element is for heating the microfluidic device from below.

In some embodiments, the reaction system further comprises an injection pump for providing gas to the plurality of gas injection valves and/or an extraction pump for providing suction to the gas extracting valves. For example, the suction may be provided by extracting gas. Optionally, the injection pump is the extraction pump. For example, a pump can have a dual function of gas injection and gas extraction. In some embodiments, the injection pump and the extraction pump are in a form of a pump assembly.

In some embodiments, the reaction system further comprises a control unit. The control unit can be in electrical communication and/or controls the plurality of gas injection valves, the plurality of gas extraction valves, the heating element, the injection pump, the extraction pump, the at least on drive module, the microfluidic device drive module, and/or the gas-flow control drive module.

In some embodiments, the reaction system further comprises a housing. The gas-flow control device, the heating element, the control unit, the injection pump, the extraction pump, the at least one drive module, the microfluidic device drive module, and/or the gas-flow control drive module can be attached and/or fixed to the housing. The housing can include, for example, at least one frame, at least shelf, at least one compartment, at least one platform, or a combination thereof.

In some embodiments, the control unit comprises a control unit interface for controlling and/or programming the control unit using a computer, a control software, a programmable software, or a combination thereof. Optionally, the reaction system comprises the computer.

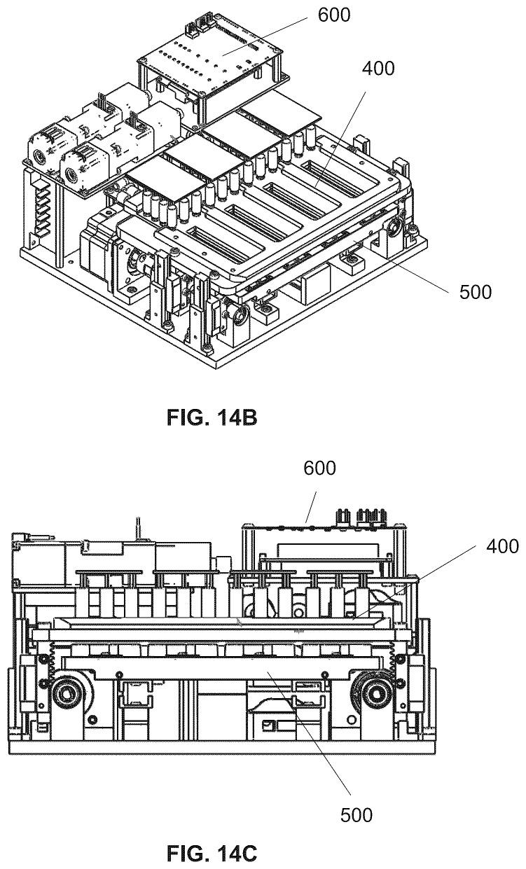

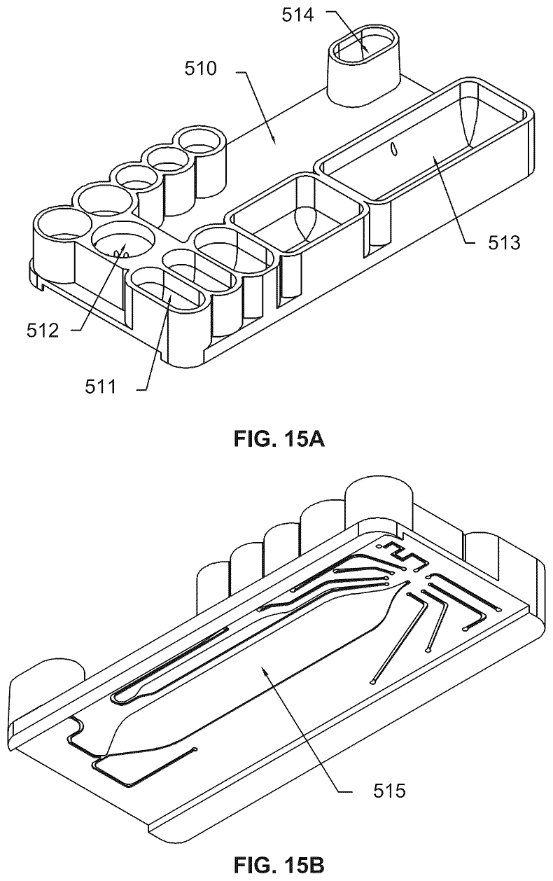

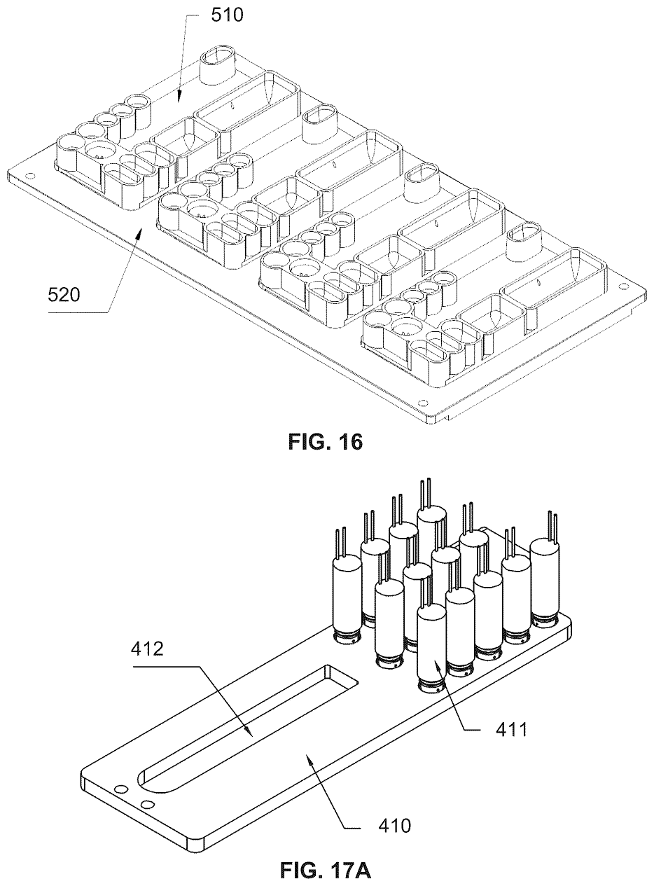

A non-limiting illustration of the present reaction system is provided in FIGS. 14A-14C, 15A-15B, 16, and 17A-17B. The illustrated representative reaction system is an integrated system, which may be useful for preparing a cell sample (such as a single cell sample). The integrated system comprises an integrated gas-path control module 400, a cell reaction module 500, and a drive module 700. The integrated gas-path control module 400 includes one or more gas-flow control device as described herein. The cell reaction module 500 includes one or more microfluidic devices as described herein. The drive module 700 is provided to achieve automatic operation for the whole process of cell preparation, such that automatic labelling of molecular tags is implemented, automation of RNA reverse transcription is achieved, and a DNA product having a certain temperature is directly produced by the device. The whole process from cell suspension to production of the DNA product is automated, thereby reducing an operation threshold for experiment staff, and simplifying operation processes.

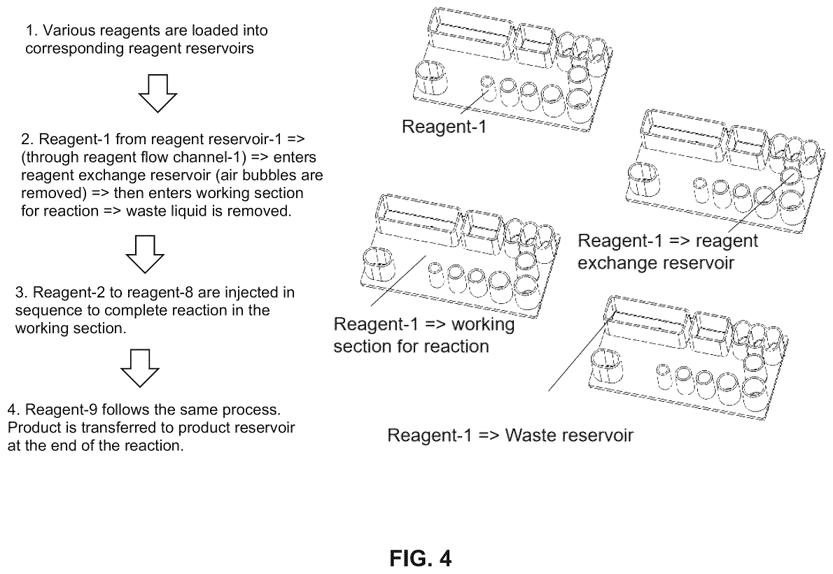

The integrated gas-path control module 400 is located above the cell reaction module 500, and the drive module 700 is divided into a horizontal movement module and a vertical movement module. The cell reaction module 500 includes a reaction platform 520 and at least two cell reaction plates 510 (also referred to as a microfluidic device herein) arranged side by side on the reaction platform 520 (FIGS. 15A and 16, 4 cell reaction plates are shown). Multiple groups of cell samples can be prepared at the same time, thereby shortening the operation time, improving preparation efficiency, and further reducing the equipment size. The integrated gas-path control module 400 comprises a gas-path platform, and at least two integrated gas-path control boards 410 (also referred to as a gas-flow control device herein) arranged side by side on the gas-path platform (FIGS. 14A and 17A, 4 gas-path control boards are shown). The locations of the cell reaction plates 510 correspond to those of the integrated gas-path control boards 410.

The reaction platform 520 is fixed on the horizontal movement module (also referred to as a microfluidic device drive module herein), and the gas-path platform is fixed on the vertical movement module (also referred to as a gas-flow control drive module herein). The horizontal movement module drives the reaction platform 520 to move away from directly below the integrated gas-path control module 400, the cell reaction plates 510 into which the reaction reagents have been injected is fixed on the reaction platform 520, then the horizontal movement module drives the reaction platform 520 to return to the original position, and the vertical movement module drives the gas-path platform to press downward such that the integrated gas-path control boards 410 are attached to the cell reaction plates 510.

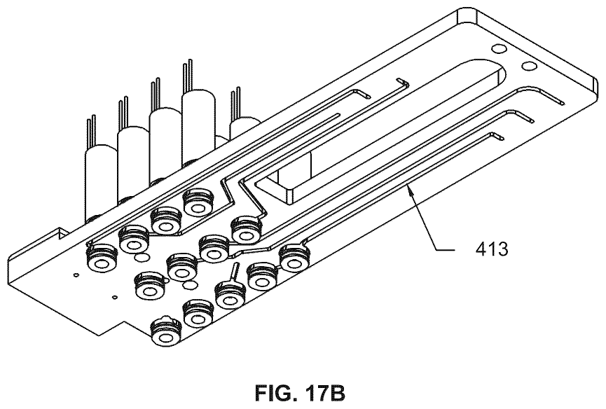

Each of the integrated gas-path control boards 410 is internally provided with at least two mutually independent driving gas channels (or microchannels) 413 (FIG. 17B). Reagent reservoirs 511 are provided on a side surface of a cell reaction plate 510 attached to the integrated gas-path control board 410, the number of the reagent reservoirs 511 is the same as the number of the driving gas channels 413 (FIGS. 15A and 17B), and each driving gas channel 413 independently communicates with a reagent reservoir 511. A reaction chamber 515 is provided on a side surface of the cell reaction plate 510 located away from the integrated gas-path control board 410 (FIG. 15B), and is in communication with the reagent reservoirs 511 into which the reaction reagents are injected in advance. A driving gas is injected into the reagent reservoirs 511 via the driving gas channels 413, such that the reaction reagents in the reagent reservoirs 511 are driven by the pressure of the driving gas to flow into the reaction chamber 515. Recesses (or grooves) are provided inside the integrated gas-path control board 410, such that various driving gas channels 413 are integrally formed. The various driving gas channels 413 are independent of each other and are not in communication with each other. The driving gas is used as a driving force for injecting the reaction reagents, so as to convey the reaction reagents in flow paths. During use, the driving gas is injected into the different driving gas channels 413, such that the reaction reagents in the different reagent reservoirs 511 are injected into the reaction chamber 515 one by one. The injection processes of the different reaction reagents are independent of each other and do not affect each other, so that flow path control processes of the reaction reagents are stable and reliable, and the cell reaction module 500 as a whole can be easily installed and occupies a reduced space.

It should be noted that in the present specific embodiment, the following two solutions, or a combination thereof, can be selected for the formation manner of the driving gas channels 413:
Solution 1: the integrated gas-path control board 410 is an integrated structure, and the driving gas channels 413 are directly provided inside the integrated gas-path control board 410 by means of casting, drilling, additive manufacturing, or the like; and
Solution 2: the integrated gas-path control board 410 is a separable structure, and is formed by stacking and attaching an upper gas-path board and a lower gas-path board together. Gas-path recesses (or grooves) are provided at an attachment surface between the upper gas-path board and/or the lower gas-path broad, and after the upper gas-path board is attached to the lower gas-path broad, the gas-path recesses are sealed to form the driving gas channels 413. Certainly, it can be understood that in this solution, the gas-path recesses can be arranged on the lower surface of the upper gas-path board or the upper surface of the lower gas-path board, or can be arranged on both the lower surface of the upper gas-path board and the upper surface of the lower gas-path board.

It should be noted that the cell reaction plate 510 is similarly provided with fluid microchannels enabling reaction reagents to flow therein. For the formation manner of the fluid microchannels, reference is made to the above description of the driving gas channels 413. That is, the cell reaction plate 510 can be an integrated structure or a separable structure. When the cell reaction plate 510 is an integrated structure, the microchannels are directly provided inside the cell reaction plate 510 by means of casting, drilling, additive manufacturing, or the like. Certainly, the cell reaction plate 510 can also be a separable structure formed by stacking and attaching an upper reaction plate and a lower reaction plate together. Flow channel recesses (or grooves) are provided at an attachment surface between the upper reaction plate and/or the lower reaction plate, and after the upper reaction plate is attached to the lower reaction plate, the flow channel recesses (or grooves) are sealed to form the microchannels.

The device for preparing a single-cell sample further comprises a control module 600 (also referred to as a control unit herein) used to independently control the horizontal movement module and the vertical movement module. The reaction platform 520 is internally provided with a heating element electrically connected to the control module 600. The control module 600 is configured to control a heating temperature of the heating element. The heating element is integrated into the reaction platform 520, and a reaction temperature is accurately controlled by means of the control module 600. When the reaction system is operating at the stage of RNA reverse transcription, the heating element provides an accurate and controllable temperature range for the reaction. The system for preparing a single-cell sample further comprises a base (also referred to as a housing herein) configured to support and fix the integrated gas-path control module 400, the cell reaction module 500, the drive module 700, and the control module 600.

The horizontal movement module comprises sliding table assemblies arranged side by side on the base, and the sliding table assemblies are fixed on a bottom surface of the reaction platform 520, and are used to support the cell reaction module 500 and to pull and move the cell reaction module 500 in a horizontal direction. Each of the sliding table assemblies includes a sliding table, a sliding table support base and a stepping motor. The sliding table is fixed on the bottom surface of the reaction platform 520, and is mounted on the sliding table support base, one end of the sliding table is connected to an output shaft of the stepping motor, and the sliding table is driven by the stepping motor to move in the horizontal direction on the sliding table support base.

The vertical movement module comprises push-rod assemblies vertically fixed to two ends of a bottom surface of the gas-path platform, each of the push-rod assemblies comprises a slide rail and a gear rack provided in the slide rail, one end of the gear rack is fixed to the edge of one end of the gas-path platform; two parallel gear shafts are disposed on a surface of the base, a drive motor is provided at one end of each gear shaft, and the gear shaft is driven by the drive motor to rotate, so as to drive the gear rack to move in the vertical direction.

The movement logic of the integrated gas-path control module 400 and the cell reaction module 500 is as follows: in an initial state, the cell reaction module 500 is located directly below the gas-path control module 400; before starting of a cell reaction, the cell reaction module 500 needs to be pulled in a horizontal direction, and an operator takes out the cell reaction plate 510, injects reaction reagents into respective reagent reservoirs 511 on the cell reaction plate 510, and subsequently fixes the cell reaction plate 510 on the reaction platform 520; the reaction platform 520 together with the cell reaction plate 510 supported thereon is moved and returned to the original position in the horizontal direction, so as to move to directly below the integrated gas-path control module 400 again; at this moment, the integrated gas-path control module 400 is pressed downward and attached to the cell reaction module 500, allowing outlet ports of the driving gas channels 413 in the integrated gas-path control board 410 to align with the corresponding reagent reservoirs 511 on the cell reaction plate 510.

The side surface of the cell reaction plate 510 attached to the integrated gas-path control board 410 is further provided with a buffer reservoir 512 (also referred to as a reagent exchange reservoir herein). The reagent reservoir 511 and the reaction chamber 515 independently communicate with the buffer reservoir 512. In a flow direction of the driving gas, the driving gas channels 413, the reagent reservoirs 511, and the buffer reservoir 512 communicate with each other in sequence. The driving gas is injected in the reagent reservoirs 511 via the driving gas channels 413, and the reaction reagents stored in the respective reagent reservoirs 511 is driven into the buffer reservoir 512 one by one, and are injected into the reaction chamber 515 via the buffer reservoir 512.

A solenoid valve 411 is provided at an inlet end of the driving gas channels 413 and is electrically connected to the control module 600. The opening degree of the solenoid valve 411 is controlled by the control module 600, so as to adjust the inlet amount of the driving gas. The solenoid valve 411 connected to the driving gas channels 413 is configured to control a flow amount of the driving gas. The reaction reagent stored in the reagent reservoir 511 is driven by the pressure of the driving gas to be pushed into the buffer reservoir 512, and the flow amount of the driving gas is adjusted by the solenoid valve 411, so as to change the injection amount of the reaction reagent into the buffer reservoir 512.

The side surface of the cell reaction plate 510 attached to the integrated gas-path control board 410 is further provided with a waste reservoir 513 (FIG. 15A) in communication with the reaction chamber 515. The integrated gas-path control board 410 is internally provided with a waste gas extraction channel (or microchannel) in communication with the waste reservoir 513. In a gas extraction direction, the reaction chamber 515, the waste reservoir 513, and the waste gas extraction channel communicate with each other in sequence. Gas extraction is performed by means of the waste liquid gas extraction channel, such that a waste obtained after completion of a reaction in the reaction chamber 515 is drawn into the waste reservoir 513. A solenoid valve 411 is provided at a gas extraction end of the waste gas extraction channel and is electrically connected to the control module 600. The opening degree of the solenoid valve 411 is controlled by the control module 600, so as to adjust a gas extraction amount.

It should be noted that the waste gas extraction channel provided in the present reaction system participates in two process steps, and specifically comprises:

Firstly, during the cell reaction, after the reaction reagents are injected into the buffer reservoir 512 from the reagent reservoirs 511, outward gas extraction is performed by means of the waste gas extraction channel. Since the waste reservoir 513, the reaction chamber 515 and the buffer reservoir 512 communicate with each other in sequence, the reaction reagents temporarily stored in the buffer reservoir 512 are drawn into the reaction chamber 515 under the action of a suction force. At this moment, it should be particularly noted that in order to prevent reaction reagents entering the reaction chamber 515 from being further drawn into the waste liquid reservoir 513, the negative pressure for gas extraction should not be excessively large.

Secondly, after the cell reaction is complete, outward gas extraction is performed again by means of the waste gas extraction channel, and a reaction waste remaining in the reaction chamber 515 is drawn into the waste reservoir 513 under the action of a suction force.

The side surface of the cell reaction plate 510 attached to the integrated gas-path control board 410 is further provided with a product reservoir 514 (FIG. 15A) in communication with the reaction chamber 515. The integrated gas-path control board 410 is internally provided with a product gas extraction channel (or microchannel) in communication with the product reservoir 514. In a gas extraction direction, the reaction chamber 515, the product reservoir 514, and the product gas extraction channel communicate with each other in sequence. Gas extraction is performed by means of the product gas extraction channel, such that a reaction product obtained in the reaction chamber 515 is drawn into the product reservoir 514.

A solenoid valve 411 is provided at a gas extraction end of the product gas extraction channel and is electrically connected to the control module 600. The opening degree of the solenoid valve 411 is controlled by the control module 600, so as to adjust a gas extraction amount. The solenoid valve 411 connected to the product gas extraction channel is configured to control a gas extraction amount. Gas extraction is performed on the product reservoir 514 by means of the product gas extraction channel, allowing the reaction product in the reaction chamber 515 to be drawn into the product reservoir 514. In order to draw the reaction product in the reaction chamber 515 completely into the product reservoir 514, the gas extraction amount needs to be strictly controlled by the solenoid valve 411.

A silicone pad is sandwiched between the integrated gas-path control board 410 and the cell reaction plate 510 and is provided with a through-hole. The integrated gas-path control board 410 and the cell reaction plate 510 communicate with each other via the through-hole. The solenoid valves 411 are concentratedly arranged on a side surface of the integrated gas-path control board 410 located away from the cell reaction plate 510 (FIG. 17A). Different types of solenoid valves 411 are provided to achieve control of the entire flow path, and the different solenoid valves 411 implement different control functions. Conventional fluid solenoid valves 411, during installation and use, have problems such as occupying a large space and requiring complex installation processes. In the present reaction system, installation locations for solenoid valves 411 are reserved on the surface of the integrated gas-path control board 410, so as to facilitate installation and removal of the solenoid valves 411. In addition, the solenoid valves 411 are integrated with the surface of the integrated gas-path control board 410, so that a fluid can pass through the flow path inside the integrated gas-path control board 410, thereby achieving control of the entire flow path. The integrated gas-path control board 410 is further provided with an observation window 412 (FIG. 17A) used to observe a reaction situation in the reaction chamber 515.

The present disclosure provides a method for carrying out a cell reaction using the device for preparing a single-cell sample provided in the above specific embodiment. The method includes:
(I) injecting the reaction reagents into the reagent reservoirs 511 in advance; driving, by the horizontal movement module, the reaction platform 520 to move away from directly below the integrated gas-path control module 400; fixing, on the reaction platform 520, the cell reaction plate 510 into which the reaction reagents have been injected; then driving the reaction platform 520 to return to the original position by means of the horizontal movement module; and driving, by the vertical movement module, the gas-path platform to press downward such that the integrated gas-path control board 410 is attached to the cell reaction plate 510;
(II) injecting a driving gas into the driving gas channels 413 via a solenoid valve 411; injecting the driving gas into the reagent reservoirs 511 along the independent driving gas channels 413 corresponding thereto; driving the reaction reagents stored in the reagent reservoirs 511 into the buffer reservoir 512 by means of the pressure of the driving gas; extracting gas from the waste reservoir 513 through a waste gas extraction channel, such that the reaction reagents in the buffer reservoir 512 are drawn into the reaction chamber to complete reagent injection; and performing a cell reaction; and
(III) after the cell reaction is complete, extracting gas from the waste reservoir 513 through the waste liquid gas extraction channel again, such that a waste in the reaction chamber 515 is drawn into the waste liquid reservoir 513; and extracting gas from the product reservoir 514 through a product gas extraction channel, such that a reaction product in the reaction chamber 515 is drawn into the product reservoir 514.

### Method of Use

Also disclosed are methods of the using the devices, modules, and systems disclosed herein. In particular, a method of performing a reaction using a microfluidic device, a gas-flow control device, a reaction module, a sample preparation device, and/or a reaction system as described herein are provided.

### Reagent Loading

Disclosed herein include methods of reagent loading In some embodiments, a method of reagent loading comprises: (a) providing a microfluidic device of the present disclosure. The method can comprise (b) loading a first reagent and a second reagent into a first reagent reservoir and a second reagent reservoir of the plurality of reagent reservoirs. The method can comprise (c1) flowing the first reagent from the first reagent reservoir into the reagent exchange reservoir through a first fluid microchannel of the plurality fluid microchannels, then into the reaction chamber, and then into the waste reservoir. The method can comprise (c2) flowing the second reagent from the second reagent reservoir into the reagent exchange reservoir chamber through a second fluid microchannel of the plurality fluid microchannels, then into the reaction chamber, and then into the waste reservoir.

In some embodiments, the method further comprises (b2) loading a third reagent into a third reagent reservoir of the plurality of reagent reservoirs. The method can comprise (c3) flowing the third reagent into the into the reagent exchange reservoir through a third fluid microchannel of the plurality fluid microchannels then into the reaction chamber, thereby a reaction occurs in the reaction chamber. The method can comprise (d) flowing one or more reaction products in the reaction chamber into the product reservoir.

In some embodiments, a method of reagent loading comprises: (a) providing the microfluidic device disclosed herein. One, one or more, or each of the plurality of reagent reservoirs can comprise a reagent. The method can comprise (c) sequentially flowing the reagent in the one, one or more, or each of the plurality of reagent reservoirs into the reagent exchange reservoir through a fluid microchannel of the plurality fluid microchannels and then into the reaction chamber. The method can comprise (d) flowing one or more waste products generated in the reaction chamber into the waste reservoir, and/or flowing one or more reaction products in the reaction chamber into the product reservoir.

The reaction chamber of the microfluidic device can comprise a microwell array comprising at least 100 microwells. In some embodiments, the first reagent comprises a plurality of cells. In some embodiments, the second reagent comprises a plurality of particles (such as beads). One, one or more, or each of the plurality of particles comprising a plurality of barcode molecules, thereby single cells and single particles (such as single beads) are loaded into microwells of the microwell array.

In some embodiments, the third reagent comprises a cell lysis reagent, an enzyme, PCR primers, and/or therapeutic compounds. In some embodiments, the reaction products comprise a plurality of barcoded target nucleic acids and/or reverse transcription products.

In some embodiments, the reaction comprises cell lysis, ligand-binding, cell-cell interaction, cell capture, nucleic acid synthesis, cellular response to a therapeutic compound, nucleic acid barcoding, reverse transcription, or a combination thereof.

In some embodiments, the microfluidic device is reversibly coupled to a gas-flow control device as described herein. In some embodiments, flowing the reagents comprises flowing the reagents using one or more gas injection valves of the plurality of gas injection valves and one or more gas extraction valves of the plurality of gas extraction valves. For example, a reagent can be injected into the reagent exchange reservoir by a positive pressure from gas injection. For example, a reagent in the reagent exchange reservoir can be drawn to the reaction chamber by a negative pressure (e.g., suction) applied through gas extraction from the waste reservoir.

In some embodiments, the gas-flow control device is comprised in a reaction module as described herein, a sample preparation device as described herein, and/or a reaction system as described herein. Optionally, flowing the reagents can comprise controlling the gas injection valves and gas extraction valves using the control unit to flow the reagents.

### Nucleic Acid Analysis

Disclosed herein include embodiments of a method of nucleic acid analysis. In some embodiments, a method of nucleic acid analysis comprises generating a plurality of barcoded target nucleic acids from a cell reaction carried out using the microfluidic device as described herein. In some embodiments, the method of nucleic acid analysis can comprise analyzing the plurality of barcoded target nucleic acids.

In some embodiments, analyzing the plurality of barcoded target nucleic acids comprises determining the sequences of the plurality of barcoded target nucleic acids. Determination of the sequence of the plurality of barcoded target nucleic acids can include, for example, library construction, sequencing, and post-sequencing analysis processes as described herein.

### Performing a Reaction

Disclosed herein include embodiments of a method of performing a reaction. As illustrated above, the present microfluidic device can be used for performing reagents exchange and/or conducting a reaction. In addition, the operations of the microfluidic device, such as reagents injection, reagent exchange, reagent mixing, waste removal, and product collection, can be controlled by pressurization and/or depressurization, which in turn can be carried out using the present gas-flow control device.

In some embodiments, a method of performing a reaction comprises (a) providing the reaction module as described herein. The method can comprise (b) loading one or more reagents into the plurality of reagent reservoirs. For each reagent reservoir of the plurality of reagent reservoirs loaded with the one or more reagents, the method can comprise (c) injecting gas into the reagent reservoir through a gas injection valve of the plurality of gas injection valves and a gas injection microchannel of the plurality of gas injection microchannels, thereby applying a positive gas pressure to the reagent reservoir, thereby injecting the one or more reagents from the reagent reservoir into the reagent exchange reservoir.

The method can include (d) transferring the one or more reagents from the reagent exchange reservoir to the reaction chamber.

For each reagent reservoir of the plurality of reagent reservoirs loaded with the one or more reagents, following (c), the method can comprise (d1) extracting gas from the waste reservoir through the waste gas extraction valve and the waste gas extraction microchannel, thereby applying a negative gas pressure to the waste reservoir, thereby transferring the one or more reagents from the reagent exchange reservoir to the reaction chamber. For each reagent reservoir of the plurality of reagent reservoirs loaded with the one or more reagents, following (c), the method can comprise (d2) extracting gas from the product reservoir through the product gas extraction valve and the product gas extraction microchannel, thereby applying a negative gas pressure to the product reservoir, thereby transferring the one or more reagents from the reagent exchange reservoir to the reaction chamber.

The method can comprise (e) allowing the one or more reagents to react in the reaction chamber. The method can comprise (f1) extracting gas from the waste reservoir through the waste gas extraction valve and the waste gas extraction microchannel, thereby applying a negative gas pressure to the waste reservoir, thereby extracting a reaction waste from the reaction chamber into the waste reservoir. The method can comprise (f2) extracting gas from the product reservoir through the product gas extraction valve and the product gas extraction microchannel, thereby applying a negative gas pressure to the product reservoir, thereby extracting a product from the reaction chamber into the product reservoir.

In some embodiments, a method of performing a reaction comprises: (a) providing a microfluidic device and a gas-flow control device disclosed herein. The method can comprise (b) loading one or more reagents into the plurality of reagent reservoirs. The method can comprise reversibly coupling the microfluidic device and the gas-flow control device. In some embodiments, the method comprises (a) providing a reaction module disclosed herein. For one, one or more, or each reagent reservoir of the plurality of reagent reservoirs loaded with the one or more reagents, the method can comprise performing (c) injecting gas into the reagent reservoir through a gas injection valve of the plurality of gas injection valves and a gas injection microchannel of the plurality of gas injection microchannels, thereby applying a positive gas pressure to the reagent reservoir, thereby injecting the one or more reagents from the reagent reservoir into the reagent exchange reservoir. The method can comprise performing (d1) extracting gas from the waste reservoir through the waste gas extraction valve and the waste gas extraction microchannel, thereby applying a negative gas pressure to the waste reservoir, thereby transferring a reagent from the reagent exchange reservoir to the reaction chamber, wherein a waste (or a reaction waste) is generated; and/or (d2) extracting gas from the product reservoir through the product gas extraction valve and the product gas extraction microchannel, thereby applying a negative gas pressure to the product reservoir, thereby transferring a reagent from the reagent exchange reservoir to the reaction chamber, wherein a product is generated. The method can comprise (e) allowing the one or more reagents in the reaction chamber to react to generate the waste or the product. The method can comprise (f1) extracting gas from the waste reservoir through the waste gas extraction valve and the waste gas extraction microchannel, thereby applying a negative gas pressure to the waste reservoir, thereby extracting the waste from the reaction chamber into the waste reservoir; and/or (f2) extracting gas from the product reservoir through the product gas extraction valve and the product gas extraction microchannel, thereby applying a negative gas pressure to the product reservoir, thereby extracting the product from the reaction chamber into the product reservoir.

As illustrated above, the present sample preparation device and/or the reaction system can provide integrated processing and automated control for conducting high-throughput reactions.

In some embodiments, a method of performing a reaction comprises (a1) providing a sample preparation device as described herein, and/or a reaction system as described herein. The method can comprise (a2) coupling each of the one or more gas-flow control devices to each of the corresponding microfluidic device of the one or more microfluidic devices. The coupling can comprise coupling each of the gas injection microchannels of the gas-flow control device to each of the plurality of reagent reservoirs of the microfluidic device, respectively. The coupling can comprise coupling the waste gas extraction microchannel of the gas-flow control device to the waste reservoir of the microfluidic device. The coupling can comprise coupling the product gas extraction microchannel of the gas-flow control device to the product reservoir of the microfluidic device.

The method can comprise (b) loading one or more reagents into the plurality of reagent reservoirs. For each reagent reservoir of the plurality of reagent reservoirs loaded with the one or more reagents, the method can comprise (c) injecting gas into the reagent reservoir through a gas injection valve of the plurality of gas injection valves and a gas injection microchannel of the plurality of gas injection microchannels, thereby applying a positive gas pressure to the reagent reservoir, thereby injecting the one or more reagents from the reagent reservoir into the reagent exchange reservoir.

The method can include (d) transferring the one or more reagents from the reagent exchange reservoir to the reaction chamber.

For each reagent reservoir of the plurality of reagent reservoirs loaded with the one or more reagents, following (c), the method can comprise (d1) extracting gas from the waste reservoir through the waste gas extraction valve and the waste gas extraction microchannel, thereby applying a negative gas pressure to the waste reservoir, thereby transferring the one or more reagents from the reagent exchange reservoir to the reaction chamber. For each reagent reservoir of the plurality of reagent reservoirs loaded with the one or more reagents, following (c), the method can comprise (d2) extracting gas from the product reservoir through the product gas extraction valve and the product gas extraction microchannel, thereby applying a negative gas pressure to the product reservoir, thereby transferring the one or more reagents from the reagent exchange reservoir to the reaction chamber.

The method can comprise (e) allowing the one or more reagents to react in the reaction chamber. The method can comprise (f1) extracting gas from the waste reservoir through the waste gas extraction valve and the waste gas extraction microchannel, thereby applying a negative gas pressure to the waste reservoir, thereby extracting a reaction waste from the reaction chamber into the waste reservoir. The method can comprise (f2) extracting gas from the product reservoir through the product gas extraction valve and the product gas extraction microchannel, thereby applying a negative gas pressure to the product reservoir, thereby extracting a product from the reaction chamber into the product reservoir.

In some embodiments, a method of performing a reaction comprises (a1) providing the sample preparation device or the sample preparation system of disclosed herein and one or more microfluidic devices of the present disclosure. The method can comprise (a2) coupling each of the one or more gas-flow control devices to one microfluidic device of the one or more microfluidic devices. The method can comprise (b) loading one or more reagents into the plurality of reagent reservoirs. For each reagent reservoir of the plurality of reagent reservoirs loaded with the one or more reagents, the method can comprise (c) injecting gas into the reagent reservoir through a gas injection valve of the plurality of gas injection valves and a gas injection microchannel of the plurality of gas injection microchannels, thereby applying a positive gas pressure to the reagent reservoir, thereby injecting the one or more reagents from the reagent reservoir into the reagent exchange reservoir. The method can comprise (d1) extracting gas from the waste reservoir through the waste gas extraction valve and the waste gas extraction microchannel, thereby applying a negative gas pressure to the waste reservoir, thereby transferring the one or more reagents from the reagent exchange reservoir to the reaction chamber, wherein a waste (or reaction waste) is performed. The method can comprise (d2) extracting gas from the product reservoir through the product gas extraction valve and the product gas extraction microchannel, thereby applying a negative gas pressure to the product reservoir, thereby transferring the one or more reagents from the reagent exchange reservoir to the reaction chamber, wherein a product (or a reaction product) is formed. The method can comprise (e) allowing the one or more reagents in the reaction chamber to react to generate a waste and/or a product. The method can comprise (f1) extracting gas from the waste reservoir through the waste gas extraction valve and the waste gas extraction microchannel, thereby applying a negative gas pressure to the waste reservoir, thereby extracting the reaction waste from the reaction chamber into the waste reservoir. The method can comprise (f2) extracting gas from the product reservoir through the product gas extraction valve and the product gas extraction microchannel, thereby applying a negative gas pressure to the product reservoir, thereby extracting a product from the reaction chamber into the product reservoir. In some embodiments, coupling the each of the one or more gas-flow control devices to one microfluidic device of the one or more microfluidic devices comprises moving the gas-flow control module and/or moving the reaction module, thereby aligning the gas-flow control module and the reaction module.

The coupling of the gas-flow control devices to the microfluidic device can be carried out manually or by machine. As illustrated above, the present reaction system is capable of moving the gas-flow control devices to the microfluidic device controlling their respective drive modules. The operation of the drive modules can be programmed and/or automated by the control unit. In some embodiments, the coupling of (a2) further comprises moving the gas-flow control module and/or moving the reaction module, thereby aligning the gas-flow control module and the reaction module.

In some embodiments, the one or more reagents comprise a cell, such as a blood cell, a nerve cell, an immune cell, a stem cell, a cancer cell, and/or other cell types disclosed herein. In some embodiments, the reaction chamber comprises a microwell array comprising at least 100 microwells, and transferring the reagent to the reaction chamber in (d) comprises partitioning the plurality of cells into the microwells, thereby at least 25% of the microwells each comprises a single cell.

In some embodiments, the one or more reagents comprise a cell lysing agent, oligonucleotides, particles comprising a plurality of barcode molecules, enzymes, PCR primers, and/or therapeutic compounds.

As illustrated above, the types of reaction that the present devices reaction modules, and reaction systems can perform is not limited. Examples of suitable reactions include, but are not limited to chemical synthesis, cell lysis, RNA reverse transcription, nucleic acid barcoding, genomic profiling, ligand binding, antibody binding, nucleic acid binding, cell signaling, cell-cell interaction, cell capture, nucleic acid synthesis, small molecule screening, and therapeutic target screening.

In some embodiments, the reaction comprise a response of a cell to an external stimuli.

In some embodiments, the reaction comprise screening a therapeutic agent, such as a small molecule or an antibody. The therapeutic agent can be one for the treatment of a disease, such as cancer.

In some embodiments, the reaction comprises a reverse transcription reaction.

In some embodiments, the reaction comprises barcoding a plurality of target nucleic acids associated with the cell using the plurality of barcode molecules to generate a plurality of barcoded target nucleic acids. The reaction can further comprise pooling the plurality of barcoded target nucleic acids as the product of the reaction.

The reaction can be automated, for example, under the control of a programmable software and/or an external computer. The reaction can be a high-throughput reaction. The present devices reaction modules, and reaction systems can allow for 2, 3, 4, 5, 6, 7, 8, 9, 10, 20, 30, 40, 50, 60, 70, 80, 90, or 100 reaction units to operate simultaneously.

### Sample Analysis

The present microfluidic device can be used for nucleic acid analysis, such as single cell nucleic acid analysis. In particular, the present device can be used to introduce a cell and a barcode molecule into partitions, and target nucleic acids associated with the cell can be barcoded by the barcode molecule to generate barcoded target nucleic acids, which can be subsequently analyzed (e.g., by sequencing) and quantified (e.g., using UMIs).

A partition as used herein can refer to a part, a portion, or a division sequestered from the rest of the parts, portions, or divisions. A partition can be formed through the use of wells, microwells, multi-well plates, microwell arrays, microfluidics, dilution, dispensing, droplets, or any other means of sequestering one fraction of a sample from another. In some embodiments, a partition is a droplet or a microwell. The barcode molecules can be attached to a particle, such as a bead. Alternatively, barcode molecules can be introduced into the partitions (e.g. microwells) by attaching or synthesizing the plurality of barcode molecules onto the surface of the partitions.

### Microwell Array

The present microfluidic device can include a partitioning element, such as a microwell array, to prepare single cell samples. The microwell array can comprise a plurality of microwells. The microwell array or the plurality of microwells of the microwell array can form a part of the reaction chamber as described herein. For example, the microwell array can be disposed on a top surface of a second layer; the top layer of the second layer and a bottom layer of a first layer together form a reaction chamber between the two surfaces for holding reagents and conducting a reaction; and surfaces and structure of the microwell array facing the first layer constitutes a part of the reaction chamber.

The microwell array can comprise different numbers of microwells in different implementations. In some embodiments, the microwell array can comprise, comprise about, comprise at least, comprise at least about, comprise at most, or comprise at most about, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 4000000, 5000000, 60000000, 70000000, 80000000, 90000000, 100000000, 200000000, 300000000, 400000000, 500000000, 600000000, 700000000, 800000000, 900000000, 1000000000, or a number or a range between any two of these values, microwells. The microwells can be arranged into rows and columns, for example. The number of microwells in a row (or a column) can be, be about, be at least, be at least about, be at most, or be at most about, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, or a number or a range between any two of these values. Adjacent rows (or columns) of microwells can be aligned or staggered, for example.

The width, length, depth (or height), radius, or diameter of a microwell of the plurality of microwells can be different in different implementations. In some embodiments, the width, length, depth (or height), radius, or diameter of a microwell of the plurality of microwells can be, be about, be at least, be at least about, be at most, or be at most about, 1 μm, 2 μm, 3 μm, 4 μm, 5 μm, 6 μm, 7 μm, 8 μm, 9 μm, 10 μm, 20 μm, 30 μm, 40 μm, 50 μm, 60 m, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 μm, 600 μm, 700 μm, 800 μm, 900 μm, 1000 μm, or a number or a range between any two of these values. For example, the width of a microwell of the plurality of microwells is 10 μm to 200 μm. As another example, the length of a microwell of the plurality of microwells can be 10 μm to 200 μm. As a further example, the depth of a microwell of the plurality of microwells can be 5 μm to 500 μm. In the non-limiting exemplary embodiment shown in FIG. 1E, the width of a microwell is 10 μm, the length of a microwell is 20 μm to 100 μm, such as 20 μm, and the depth of a microwell is 5 μm to 10 μm. The shape of a microwell can be different in different implementations. In some embodiments, a microwell of the plurality of microwells has a circular, elliptical, square, rectangular, triangular, or hexagonal shape.

The volume of one, one or more, or each, of the plurality of partitions can be different in different embodiments. The volume of one, one or more, or each, of the plurality of partitions can be, be about, be at least, be at least about, be at most, or be at most about, 1 nm³, 2 nm³, 3 nm³, 4 nm³, 5 nm³, 6 nm³, 7 nm³, 8 nm³, 9 nm³, 10 nm³, 20 nm³, 30 nm³, 40 nm³, 50 nm³, 60 nm³, 70 nm³, 80 nm³, 90 nm³, 100 nm³, 200 nm³, 300 nm³, 400 nm³, 500 nm³, 600 nm³, 700 nm³, 800 nm³, 900 μm³, 1000 nm³, 10000 nm³, 100000 μm³, 1000000 nm³, 10000000 nm³, 100000000 μm³, 1000000000 nm³, 2 μm³, 3 μm³, 4 μm³, 5 μm³, 6 μm³, 7 μm³, 8 μm³, 9 μm³, 10 μm³, 20 μm³, 30 μm³, 40 μm³, 50 μm, 60 μm, 70 μm³, 80 μm³, 90 μm³, 100 μm³, 200 μm³, 300 μm³, 400 μm³, 500 μm³, 600 μm³, 700 μm³, 800 μm³, 900 μm³, 1000 μm³, 10000 μm³, 100000 μm³, 1000000 μm³, or a number or a range between any two of these values. The volume of one, one or more, or each, of the plurality of partitions can be, be about, be at least, be at least about, be at most, or be at most about, 1 nanolieter (nl), 2 nl, 3 nl, 4 nl, 5 nl, 6 nl, 7 nl, 8 nl, 9 nl, 10 nl, 11 nl, 12 nl, 13 nl, 14 nl, 15 nl, 16 nl, 17 nl, 18 nl, 19 nl, 20 nl, 21 nl, 22 nl, 23 nl, 24 nl, 25 nl, 26 nl, 27 nl, 28 nl, 29 nl, 30 nl, 31 nl, 32 nl, 33 nl, 34 nl, 35 nl, 36 nl, 37 nl, 38 nl, 39 nl, 40 nl, 41 nl, 42 nl, 43 nl, 44 nl, 45 nl, 46 nl, 47 nl, 48 nl, 49 nl, 50 nl, 51 nl, 52 nl, 53 nl, 54 nl, 55 nl, 56 nl, 57 nl, 58 nl, 59 nl, 60 nl, 61 nl, 62 nl, 63 nl, 64 nl, 65 nl, 66 nl, 67 nl, 68 nl, 69 nl, 70 nl, 71 nl, 72 nl, 73 nl, 74 nl, 75 nl, 76 nl, 77 nl, 78 nl, 79 nl, 80 nl, 81 nl, 82 nl, 83 nl, 84 nl, 85 nl, 86 nl, 87 nl, 88 nl, 89 nl, 90 nl, 91 nl, 92 nl, 93 nl, 94 nl, 95 nl, 96 nl, 97 nl, 98 nl, 99 nl, 100 nl, or a number or a range between any two of these values. For example, the volume of one, one or more, or each, of the plurality of partitions is about 1 nm³ to about 1000000 μm³.

The microwell array comprising a plurality of microwells can be formed from any suitable material as will be understood by a person skilled in the art. In some embodiments, a microwell array comprising a plurality of microwells can be formed from a material selected from the group consisting of silicon, glass, ceramic, elastomers such as polydimethylsiloxane (PDMS) and thermoset polyester, thermoplastic polymers such as polystyrene, polycarbonate, poly(methyl methacrylate) (PMMA), poly-ethylene glycol diacrylate (PEGDA), Teflon, polyurethane (PU), composite materials such as cyclic-olefin copolymer, and combinations thereof.

Microwells can be introduced with samples, free reagents, and/or reagents encapsulated in microcapsules. The reagents can comprise restriction enzymes, ligase, polymerase, fluorophores, barcode molecules, oligonucleotide probes, adapters, buffers, dNTPs, ddNTPs, and other reagents required for performing the methods described herein. Samples and reagents can flow into the reaction chamber of the microfluidic device and delivered to the microwell array, and the waste can be removed (e.g., pulled into a waste reservoir).

### Target Nucleic Acids and Cells

The cells can be associated with target nucleic acids. For example, a cell can comprise a target nucleic acid (e.g., mRNA) or can be labeled with a target nucleic acid (e.g., directly, or indirectly through a binding moiety, such as an antibody conjugated with the nucleic acid). The target nucleic acids associated with the cell can be from, on the surface of, or binding to the surface of the cell. A target nucleic acid can have a sequence (e.g., an mRNA sequence, excluding the poly(A) tail).

The cells can be obtained from any organism of interest. A cell can be a mammalian cell, and particularly a human cell such as T cells, B cells, natural killer cells, stem cells, cancer cells, or any cells the functionality of which can be affected by the presence of other cells (e.g. cells involved in cell-cell interaction).

Cells described herein can be obtained from a cell sample. A cell sample comprising cells can be obtained from any source including a clinical sample and a derivative thereof, a biological sample and a derivative thereof, a forensic sample and a derivative thereof, and a combination thereof. A cell sample can be collected from any bodily fluids including, but not limited to, blood, urine, serum, lymph, saliva, anal, and vaginal secretions, perspiration and semen of any organism. A cell sample can be products of experimental manipulation including purification, cell culturation, cell isolation, cell separation, cell quantification, sample dilution, or any other cell sample processing approaches. A cell sample can be obtained by dissociation of any biopsy tissues of any organism including, but not limited to, skin, bone, hair, brain, liver, heart, kidney, spleen, pancreas, stomach, intestine, bladder, lung, esophagus.

The target nucleic acids associated with the cell can comprise deoxyribonucleic acid (DNA), ribonucleic acid (RNA), and/or any combination or hybrid thereof. The target nucleic acids can be single-stranded or double-stranded, or contain portions of both double-stranded or single-stranded sequences. The target nucleic acids can contain any combination of nucleotides, including uracil, adenine, thymine, cytosine, guanine, inosine, xanthine, hypoxanthine, isocytosine, isoguanine and any nucleotide derivative thereof. As used herein, the term “nucleotide” may include naturally occurring nucleotides and nucleotide analogs, including both synthetic and naturally occurring species. The target nucleic acids can be genomic DNA (gDNA), mitochondrial DNA (mtDNA), messenger RNA (mRNA), ribosomal RNA (rRNA), transfer RNA (tRNA), nuclear RNA (nRNA), small interfering RNA (siRNA), small nuclear RNA (snRNA), small nucleolar RNA (snoRNA), small Cajal body-specific RNA (scaRNA), microRNA (miRNA), double stranded (dsRNA), ribozyme, riboswitch or viral RNA, or any nucleic acids that may be obtained from a sample.

### Particles

The barcode molecules introduced into the partitions (such as microwells) can be associated with a particle. The particles with barcode molecules can be injected into the reaction chamber of the present microfluidic device according to the process of using the device as described herein. The particles can provide a surface upon which molecules, such as oligonucleotides, can be synthesized or attached. In some embodiments, a particle comprises, comprises about, comprises at least, comprises at least about, comprises at most, or comprises at most about 1, 5, 10, 50, 100, 1000, 10000, 20000, 30000, 40000, 50000, 60000, 70000, 80000, 90000, 100000, 200,000, 300,000, 400,000, 500,000, 600,000, 700,000, 800,000, 900,000, 1000000, 2000000, 3000000, 4000000, 5000000, 6000000, 7000000, 8000000, 9000000, 1000000, 20000000, 50000000, 100000000, 200000000, 30000000M, 400000000, 500000000, 600000000, 700000000, 800000000, 900000000, 1000000000, or a number or a range between any two of these values, barcode molecules. The attachment of barcode molecules to the particle can be covalent or non-covalent via non-covalent bonds such as ionic bonds, hydrogen bonds, or van der Waals interactions. The attachment can be direct to the surface of a particle or indirect through other oligonucleotide sequences attached to the surface of a particle.

A particle can be dissolvable, degradable, or disruptable. A particle can be a gel particle such as a hydrogel particle. In some embodiments, the gel particle is degradable upon application of a stimulus. The stimulus can comprise a thermal stimulus, a chemical stimulus, a biological stimulus, a photo-stimulus, or a combination thereof.

A particle can be a solid particle and/or a magnetic particle. In some embodiments, the particle is a magnetic particle. The magnetic particle can comprise a paramagnetic material coated or embedded in the magnetic particle (e.g. on a surface, in an intermediate layer, and/or mixed with other materials of the magnetic particle). A paramagnetic material refers to a material having a magnetic susceptibility slightly greater than 1 (e.g. between about 1 and about 5). A magnetic susceptibility is a measure of how much a material can become magnetized in an applied magnetic field. Paramagnetic materials include, but not limited to, magnesium, molybdenum, lithium, aluminum, nickel, tantalum, titanium, iron oxide, gold, copper, or a combination thereof.

In some embodiments, the magnetic particle comprising barcode molecules can be immobilized or retained in a partition (such as a microwell) by an external magnetic field, thereby retaining the barcode molecules in a partition. The magnetic particle comprising barcode molecules can be mobilized or released when the external magnetic field is removed.

In some embodiments, a particle can be immobilized or retained in a partition (e.g., a microwell) through an interaction between two members of a binding pair. For example, the partition (e.g., microwell) can be coated with a capture moiety (e.g., a member of a binding pair) capable of binding with a binding moiety (the other member of the binding pair) comprised in or conjugated to a particle, such that the binding of the two moieties results in the attachment of the particle to the partition (e.g., microwell), thereby immobilizing or retaining the particle in the partition. For example, the surface of a partition (e.g., microwell) can be coated with streptavidin. The biotinylated particle can be attached to the surface of the partition (e.g., microwell) via streptavidin-biotin interaction.

Particles can be of uniform size or heterogeneous size. In some embodiments, the particles have a diameter of about, at least, at least about, at most, or at most about, 1 μm, 5 μm, 10 μm, 20 μm, 30 μm, 40 μm, 45 μm, 50 μm, 60 μm, 65 μm, 70 μm, 75 μm, 80 μm, 90 μm, 100 μm, 250 μm, 500 μm, or 1 mm.

In some embodiments, a particle can be sized such that at most one particle, not two particles, can fit one partition. A size or dimension (e.g., length, width, depth, radius, or diameter) of a particle can be different in different embodiments. In some embodiments, a size or dimension of one, or each, particle is, is about, is at least, is at least about, is at most, or is at most about, 1 nanometer (nm), 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, 800 nm, 810 nm, 820 nm, 830 nm, 840 nm, 850 nm, 860 nm, 870 nm, 880 nm, 890 nm, 900 nm, 910 nm, 920 nm, 930 nm, 940 nm, 950 nm, 960 nm, 970 nm, 980 nm, 990 nm, 1000 nm, 2 micrometer (μm), 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 μm, or a number or a range between any two of these values. For example, a size or dimension of one, or each, particle is about 1 nm to about 100 μm. As another example, the particle can have a dimension about 10 μm to about 100 μm. As another example, the particle can have a dimension about 30 μm.

The volume of one, or each, particle can be different in different embodiments. The volume of one, or each, particle can be, be about, be at least, be at least about, be at most, or be at most about, 1 nm³, 2 nm³, 3 nm³, 4 nm³, 5 nm³, 6 nm³, 7 nm³, 8 nm³, 9 nm³, 10 nm³, 20 nm³, 30 nm³, 40 nm³, 50 nm³, 60 nm³, 70 nm³, 80 nm³, 90 nm³, 100 nm³, 200 nm³, 300 nm³, 400 nm³, 500 nm³, 600 nm³, 700 nm³, 800 nm³, 900 μm³, 1000 nm³, 10000 nm³, 100000 μm³, 1000000 nm³, 1000000 nm³, 100000000 μm³, 1000000000 nm³, 2 μm³, 3 μm³, 4 μm³, 5 μm³, 6 μm³, 7 μm³, 8 μm³, 9 μm³, 10 μm³, 20 μm³, 30 μm³, 40 μm³, 50 μm³, 60 μm³, 70 μm³, 80 μm³, 90 μm³, 100 μm³, 200 μm³, 300 μm³, 400 μm³, 500 μm³, 600 μm³, 700 μm³, 800 μm³, 900 μm³, 1000 μm³, 10000 m³, 100000 μm³, 1000000 μm³, or a number or a range between any two of these values. The volume of one, or each, particle can be, be about, be at least, be at least about, be at most, or be at most about, 1 nanolieter (nL), 2 nL, 3 nL, 4 nL, 5 nL, 6 nL, 7 nL, 8 nL, 9 nL, 10 nL, 11 nL, 12 nL, 13 nL, 14 nL, 15 nL, 16 nL, 17 nL, 18 nL, 19 nL, 20 nL, 21 nL, 22 nL, 23 nL, 24 nL, 25 nL, 26 nL, 27 nL, 28 nL, 29 nL, 30 nL, 31 nL, 32 nL, 33 nL, 34 nL, 35 nL, 36 nL, 37 nL, 38 nL, 39 nL, 40 nL, 41 nL, 42 nL, 43 nL, 44 nL, 45 nL, 46 nL, 47 nL, 48 nL, 49 nL, 50 nL, 51 nL, 52 nL, 53 nL, 54 nL, 55 nL, 56 nL, 57 nL, 58 nL, 59 nL, 60 nL, 61 nL, 62 nL, 63 nL, 64 nL, 65 nL, 66 nL, 67 nL, 68 nL, 69 nL, 70 nL, 71 nL, 72 nL, 73 nL, 74 nL, 75 nL, 76 nL, 77 nL, 78 nL, 79 nL, 80 nL, 81 nL, 82 nL, 83 nL, 84 nL, 85 nL, 86 nL, 87 nL, 88 nL, 89 nL, 90 nL, 91 nL, 92 nL, 93 nL, 94 nL, 95 nL, 96 nL, 97 nL, 98 nL, 99 nL, 100 nL, or a number or a range between any two of these values. For example, the volume of one, or each, particle is about 1 nm³ to about 1000000 μm³.

The number of particles introduced into a plurality of partitions can be different in different embodiments. In some embodiments, the number of particles introduced into a plurality of partitions is, is about, is at least, is at least about, is at most, or is at most, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 60000, 700000, 80000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these values. For example, the number of particles introduced into a plurality of partitions (e.g. microwells) can be at least 80,000 particles.

In some embodiments, particles are introduced to the partitions such that the percentage of partitions each occupied with one particle is, is about, is at least, is at least about, is at most, or is at most about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two of these values. For example, at least 80% of the plurality of partitions can be each occupied with one particle.

In some embodiments, particles are introduced to the partitions such that the percentage of partitions with no particle is, is about, is at least, is at least about, is at most, or is at most about, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or a number or a range between any two of these values. For example, at most 20% of the plurality of partitions contain no particle.

Introducing Barcode Molecules into Partitions without Using a Particle

In some embodiments, the barcode molecules are introduced into the partitions without using a particle. In some embodiments, the barcode molecules can be introduced into the partitions (e.g. microwells) by attaching or synthesizing the plurality of barcode molecules onto the surface of the partitions.

### Partitioning Cells

The cells can be loaded into the reaction chamber of the present microfluidic device according to the process of using the device as described herein, and be partitioned into a plurality of partitions (e.g., a plurality of microwells in a microwell array disposed in the reaction chamber) in the reaction chamber. For studies of cell-cell interaction, two or more different cells can be loaded into the reaction chamber and partitioned. The two or more difference cells can be loaded as one reagent and partitioned together (e.g. co-partitioned). Alternatively, the two or more difference cells can be loaded as different reagents and partitioned separately.

As a result of partitioning, the percentage of the plurality of partitions comprising a desired number of cell(s) (e.g., a single cell, or two cells cell-cell interaction analysis) and optionally a single particle can be different in different embodiments. In some embodiments, the percentage of the plurality of partitions comprising the desired number of cells and optionally a single particle is, is about, is at least, is at least about, is at most, or is at most about, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two of these values. For example, at least 10% of the plurality of partitions comprise a single cell and optionally a single particle

The percentage of the plurality of partitions comprising no cell can be different in different embodiments. In some embodiments, the percentage of the plurality of partitions comprising no cell is, is about, is at least, is at least about, is at most, or is at most about, %, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or a number or a range between any two of these values. For example, at most 50% of partitions of the plurality of partitions can comprise no cell of the plurality of cells.

The percentage of the plurality of partitions comprising more than the desired number of cell(s) can be different in different embodiments. In some embodiments, the percentage of the plurality of partitions comprising more than two cells is, is about, is at least, is at least about, is at most, or is at most about, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or a number or a range between any two of these values. For example, at most 10% of partitions of the plurality of partitions can comprise more than two cells of the plurality of cells.

### Barcode Molecules

Barcode molecules (e.g., barcode molecules attached to beads) can be partitioned, for example, in microwells. The term “barcode” as used herein generally can be a verb or a noun. When used as a noun, the term “barcode” or “barcode molecule” refers to a label that can be attached to a polynucleotide, or any variant thereof, to convey information about the polynucleotide. For example, a barcode can be a polynucleotide sequence attached to all fragments of the target nucleic acids associated with the first cell and/or the second cell in the partition. The barcode can then be sequenced alone or with the fragments of the target nucleic acids associated with the first cell and/or the second cell. The presence of the same barcode on multiple sequences or different barcodes on different sequences can provide information about the cell origin and/or the molecular origin of the sequences. When used as a verb, the term “barcode” refers to a process of attaching a barcode or a barcode molecule to a target nucleic acid associated with the first cell and/or the second cell.

Barcode molecules can be generated from a variety of different formats, including pre-designed polynucleotide barcodes, randomly synthesized barcode sequences, microarray-based barcode synthesis, random N-mers, or combinations thereof as will be understood by a person skilled in the art.

In some embodiments, the barcode molecules comprise, comprise about, comprise at least, comprise at least about, comprise at most, or comprise at most about 1, 5, 10, 50, 100, 1000, 10000, 20000, 30000, 40000, 50000, 60000, 70000, 80000, 90000, 100000, 200,000, 300,000, 400,000, 500,000, 600,000, 700,000, 800,000, 900,000, 1000000, 2000000, 3000000, 4000000, 5000000, 6000000, 70000000, 8000000, 9000000, 10000000, 20000000, 50000000, 100000000, 200000000, 300000000, 400000000, 500000000, 600000000, 700000000, 800000000, 900000000, 1000000000, or a number or a range between any two of these values, barcode molecules.

A barcode molecule (or a segment of a barcode molecule, such as a molecular barcode or a cell barcode) can be in any suitable length. In some embodiments, a barcode molecule (or a segment of a barcode molecule) can be about 2 to about 500 nucleotides in length, about 2 to about 100 nucleotides in length, about 2 to about 50 nucleotides in length, about 2 to about 40 nucleotides in length, about 4 to about 20 nucleotides in length, or about 6 to 16 nucleotides in length.

In some embodiments, a barcode molecule (or a segment of a barcode molecule) is about, at least, at least about, at most, or at most about 2, 3, 4, 5, 6, 7, 8, 9, 10, 11, 12, 13, 14, 15, 16, 17, 18, 19, 20, 25, 30, 35, 40, 45, 50, 55, 60, 65, 70, 85, 90, 95, 100, 150, 200, 250, 300, 400, or 500 nucleotides in length, or a number or a range between any two of these values.

The barcode molecules used herein can comprise a cell barcode and a molecular barcode (e.g. a unique molecular identifier (UMI)). A barcode molecule can also comprise additional sequences, such as a target binding sequence or region capable of hybridizing to target nucleic acids (e.g. poly(dT) sequence), other recognition or binding sequences, a template switching oligonucleotide (e.g., GGG, such as rGrGrG), and primer sequences (e.g. sequencing primer sequence, such as Read 1 or a PCR primer sequence) for subsequent processing (e.g. PCR amplification) and/or sequencing.

The configuration of the various sequences comprised in a barcode molecule (e.g. cell barcode sequence, UMI, primer sequence, target binding sequence or region, and/or any additional sequences) can vary depending on, for example, the particular configuration desired and/or the order in which the various components of the sequence are added as will be understood to a person skilled in the art. In some embodiments, a barcode molecule has a configuration of 5′-primer sequence-cell barcode-UMI-target binding sequence-3′. In some embodiments, a barcode molecule has a configuration of 5-primer sequence-cell barcode-UMI-template switching oligonucleotide-3′.

Cell Barcode

In some embodiments, a barcode molecular can comprise a cell barcode. Cell barcodes can be used to identify the barcoded nucleic acids originate from the cell (or the same partition). Barcoded nucleic acids that originate from the cell (or the same partition) can have an identical cell barcode. A cell barcode can be referred to as a partition specific barcode, such as a microwell specific barcode. The cell barcodes of the barcode molecules in a partition can be identical or different.

In some embodiments, the cell barcodes can serve to track the target nucleic acids associated with the cell throughout the processing (e.g., location of the cells in a plurality of partitions, such as microwells) when the cell barcode associated with the target nucleic acids is determined during sequencing. Targeted

The number (or percentage) of barcode molecules introduced in a partition with cell barcodes having an identical sequence can be different in different embodiments. In some embodiments, the number of barcode molecules introduced in a partition with cell barcodes having an identical sequence is, is about, is at least, is at least about, is at most, or is at most about, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these values. In some embodiments, the percentage of barcode molecules introduced in a partition with cell barcodes having an identical sequence is, is about, is at least, is at least about, is at most, or is at most about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 100%, or a number or a range between any two of these values. For example, the cell barcodes of at least two barcode molecules introduced in a partition comprise an identical sequence.

A cell barcode can be unique (or substantially unique) to a partition. The number of unique cell barcode sequences can be different in different embodiments. In some embodiments, the number of unique cell barcode sequences is, is about, is at least, is at least about, is at most, or is at most about, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these values. In some embodiments, the percentage of unique cell barcode sequences is, is about, is at least, is at least about, is at most, or is at most about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 100%, or a number or a range between any two of these values, of the cell barcode sequences of the barcode molecules introduced in a partition. For example, the cell barcodes of barcode molecules introduced in two partition can comprise different sequences.

In some embodiments, barcode molecules are introduced to the plurality of partitions such that different sets of a plurality of barcode molecules introduced in different partitions have different cell barcode and a same set of plurality of barcode molecules introduced in a same partition have same cell barcode. For example, target nucleic acids associated with a cell in a partition will be barcoded with the same cell barcode.

The length of a cell barcode of a barcode molecule (or a cell barcode of each barcode molecule or all cell barcodes of the plurality of barcode molecules) can be different in different embodiments. In some embodiments, a cell barcode of a barcode molecule (or each cell barcode of each barcode molecule or all cell barcodes of the plurality of barcode molecules) is, is about, is at least, is at least about, is at most, or is at most about, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values, nucleotides in length.

Molecular Barcodes

In some embodiments, a barcode molecular can comprise a molecular barcode or molecular label, molecular barcodes are unique molecule identifiers (UMIs). Molecular barcodes can be used to identify molecular origins of the barcoded nucleic acids. Molecular barcodes (e.g., UMIs) are short sequences used to uniquely tag each molecule in a sample in some embodiments. The molecular barcodes of the barcode molecules partitioned into a partition can be identical or different.

In some embodiments, the molecular barcodes of the plurality of barcode molecules are different. The number (or percentage) of molecular barcodes of barcode molecules introduced in a partition (such as a microwell) with different sequences can be different in different embodiments. In some embodiments, the number of molecular barcodes of barcode molecules introduced in a partition with different sequences is, is about, is at least, is at least about, is at most, or is at most about, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 200000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these values. In some embodiments, the percentage of molecular barcodes of barcode molecules introduced in a partition with different sequences is, is about, is at least, is at least about, is at most, or is at most about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 100%, or a number or a range between any two of these values. For example, the molecular barcodes of two barcode molecules of the plurality of barcode molecules introduced in a partition can comprise different sequences.

The number of barcode molecules introduced in a partition with molecular barcodes having an identical sequence can be different in different embodiments. In some embodiments, the number of barcode molecules introduced in a partition with molecular barcodes having an identical sequence is, is about, is at least, is at least about, is at most, or is at most about, 1, 2, 3, 4, 5, 6, 7, 8, 9, 10, 20, 30, 40, 50, 60, 70, 80, 90, 100, or a number or a range between any two of these values. For example, the molecular barcodes of two barcode molecules introduced in a partition can comprise an identical sequence.

The number of unique molecular barcode sequences can be different in different embodiments. In some embodiments, the number of unique molecular barcode sequences is, is about, is at least, is at least about, is at most, or is at most about, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 6000000, 7000000, 800000, 900000, 1000000, 2000000, 300000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these values.

The length of a molecular barcode of a barcode molecule (or a molecular barcode of each barcode molecule) can be different in different embodiments. In some embodiments, a UMI of a barcode molecule (or a UMI of each barcode molecule) is, is about, is at least, is at least about, is at most, or is at most about, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values, nucleotides in length.

Primer Sequence

In some embodiments, a barcode molecule can comprise a primer sequence. The primer sequence can be a sequencing primer sequence (or a sequencing primer binding sequence) or a PCR primer sequence (or PCR primer binding sequence). For example, the sequencing primer is a Read 1 sequence.

The length of the primer sequence can be different in different embodiments. In some embodiments, the primer sequence is 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values, nucleotides in length. The number (or percentage) of barcode molecules attached to a microwell each comprising a primer sequence (or each comprising an identical primer sequence) can be different in different embodiments. In some embodiments, the number of barcode molecules attached to a microwell each comprising a primer sequence is, is about, is at least, is at least about, is at most, or is at most about, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these values. In some embodiments, the percentage of barcode molecules attached to a microwell each comprising a primer sequence (or each comprising an identical primer sequence) is, is about, is at least, is at least about, is at most, or is at most about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 100%, or a number or a range between any two of these values.

Target Binding Sequence

In some embodiments, a barcode molecule can comprise a target binding sequence or region capable of hybridizing to the target nucleic acids, a particular type of target nucleic acids (e.g. mRNA), and/or specific target nucleic acids (e.g. specific gene of interest).

The length of a target binding sequence can be different in different embodiments. In some embodiments, a target binding sequence is, is about, is at least, is at least about, is at most, or is at most about, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values, nucleotides in length. The target binding sequence can be 12-18 deoxythymidines in length. In some embodiments, the target binding sequence can be 20 nucleotides or longer to enable their annealing in reverse transcription reactions at higher temperatures as will be understood by a person skilled in the art.

In some embodiments, barcode molecules comprising target binding sequences can be introduced into the partitions together with other reagents such as the reverse transcription reagents. The number of the barcode molecules introduced into a partition comprising a target binding sequence can be different in different embodiments. In some embodiments, the number of barcode molecules introduced into a partition comprising a target binding sequence (e.g., poly(dT) sequence) is, is about, is at least, is at least about, is at most, or is at most about, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 600000000, 70000000, 80000000, 90000000, 100000000, 200000000, 300000000, 400000000, 500000000, 600000000, 700000000, 800000000, 900000000, 1000000000, or a number or a range between any two of these values.

In some embodiments, the target binding sequence can be on a 3′ end of a barcode molecule of the plurality of barcode molecules introduced in a partition. Barcode molecules each comprising a poly(dT) target binding sequence can be used to capture (e.g., hybridize to) 3′ end of polyadenylated mRNA transcripts in a target nucleic acid for a downstream 3′ gene expression library construction.

In some embodiments, the target binding sequence can comprise a poly(dT) sequence which is a single-stranded sequence of deoxythymidine (dT) used for first-strand cDNA synthesis catalyzed by reverse transcriptase. In some embodiments, the target binding sequence comprises a poly(dT) sequence can be introduced into the partitions as extension primers to synthesize the first-strand cDNA using the target nucleic acid (e.g. RNA) as a template.

In some embodiments, the poly(dT) of the barcode molecules introduced into a partition can be identical (e.g., same number of dTs). In some embodiments, the poly(dT) of the barcode molecules introduced into a partition can be different (e.g. different numbers of dTs). The percentage of the barcode molecules of the plurality of barcode molecules introduced into a partition with an identical poly(dT) sequence can be different in different embodiments. In some embodiments, the percentage of the barcode molecules of the plurality of barcode molecules introduced into a partition with an identical poly(dT) sequence is, is about, is at least, is at least about, is at most, is at most about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 100%, or a number or a range between any two of these values.

In some embodiments, the target binding regions of all barcode molecules of the plurality of barcode molecules comprise poly(dT) capable of hybridizing to poly(A) tails of mRNA molecules (or poly(dA) regions or tails of DNA). In some embodiments, the target binding regions of some barcode molecules of the plurality of barcode molecules comprise gene-specific or target-specific primer sequences. For example, a barcode molecule of the plurality of barcode molecules can also comprise a target binding region capable of hybridizing to a specific target nucleic acid associated with the cell, thereby capturing specific targets or analytes of interest. For example, the target binding region capable of hybridizing to a specific target nucleic acid can be a gene-specific primer sequence. The gene-specific primer sequences can be designed based on known sequences of a target nucleic acid of interest. The gene-specific primer sequences can span a nucleic acid region of interest, or adjacent (upstream or downstream) of a nucleic acid region of interest.

The length of a gene-specific primer sequence can be different in different embodiments. In some embodiments, a gene-specific primer sequence is, is about, is at least, is at least about, is at most, or is at most about, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values, nucleotides in length. For example, a gene-specific primer sequence is at least 10 nucleotides in length.

The number of the barcode molecules introduced into a partition comprising a gene-specific primer sequence can be different in different embodiments. In some embodiments, the number of barcode molecules introduced into a partition comprising a gene-specific primer sequence is, is about, is at least, is at least about, is at most, or is at most about, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 2000000, 300000000, 400000000, 50000000, 600000000, 7040000000, 800000000, 900000000, 1000000000, or a number or a range between any two of these values.

In some embodiments, the barcode molecules introduced into a partition can comprise a set of different gene-specific primer sequences each capable of binding to a specific target nucleic acid sequence.

The number of different gene-specific primer sequences of the barcode molecules introduced into a partition can be different in different embodiments. In some embodiments, the number of different gene-specific primer sequences of the barcode molecules introduced into a partition is, is about, is at least, is at least about, is at most, or is at most about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, 2000, 3000, 4000, 5000, 6000, 7000, 8000, 9000, 10000, 20000, 50000, 1000000, or a number or a range between any two of these values.

Accordingly, the number of target nucleic acids of interest (e.g. genes of interest) that the barcode molecules introduced into a partition are capable of binding can be different in different embodiments. In some embodiments, the number of target nucleic acids of interest (e.g. genes of interest) the barcode molecules introduced into a partition are capable of binding is, is about, is at least, is at least about, is at most, or is at most about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, 2000, 3000, 4000, 5000, 6000, 7000, 8000, 9000, 10000, 20000, 50000, 1000000, or a number or a range between any two of these values. One barcode molecule introduced into a partition can bind to a molecule (or a copy) of a target nucleic acid. Barcode molecules introduced into a partition can bind to molecules (or copies) of a target nucleic acid or a plurality of target nucleic acids.

In some embodiments, the barcode molecules of the plurality of barcode molecules can each comprise a poly(dT) sequence, a gene-specific primer sequence, and/or both. The poly(dT) sequence and the gene-specific primer sequence can be on a same barcode molecule or different barcode molecules of the plurality of barcode molecules introduced into a partition.

The ratio of the number of barcode molecules introduced into a partition comprising a poly(dT) sequence and the number of barcode molecules introduced into a partition comprising a gene-specific primer sequence can be different in different embodiments. In some embodiments, the ratio is, is about, is at least, is at least about, is at most, is at most about, 1:100, 1:99, 1:98, 1:97, 1:96, 1:95, 1:94, 1:93, 1:92, 1:91, 1:90, 1:89, 1:88, 1:87, 1:86, 1:85, 1:84, 1:83, 1:82, 1:81, 1:80, 1:79, 1:78, 1:77, 1:76, 1:75, 1:74, 1:73, 1:72, 1:71, 1:70, 1:69, 1:68, 1:67, 1:66, 1:65, 1:64, 1:63, 1:62, 1:61, 1:60, 1:59, 1:58, 1:57, 1:56, 1:55, 1:54, 1:53, 1:52, 1:51, 1:50, 1:49, 1:48, 1:47, 1:46, 1:45, 1:44, 1:43, 1:42, 1:41, 1:40, 1:39, 1:38, 1:37, 1:36, 1:35, 1:34, 1:33, 1:32, 1:31, 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, or a number or a range between any two of these values.

Template Switching Oligonucleotide

In some embodiments, a barcode molecule (or each barcode molecule of the plurality of barcode molecules) can be a template switching oligonucleotide (TSO). A primer comprising a target binding region, such as a poly(dT) sequence, can hybridize to a target nucleic acid (e.g., an mRNA) and be extended by, for example, reverse transcription to generate an extended primer comprising a reverse complement of the target nucleic acid, or a portion thereof (e.g., cDNA). The extended primer or cDNA can be further extended to include the reverse complement of a TSO oligonucleotide or barcode molecule. The resulting barcoded nucleic acid includes the barcodes of the barcode molecule on the 3′-end.

In some embodiments, a barcode molecule is not a template switching oligonucleotide. A barcode molecule comprising a target binding region, such as a poly(dT) sequence, can hybridize to a target nucleic acid (e.g., an mRNA) and be extended by, for example, reverse transcription to generate an extended primer comprising a reverse complement of the target nucleic acid, or a portion thereof (e.g., cDNA). The extended primer or cDNA can be further extended to include the reverse complement of a TSO oligonucleotide. The resulting barcoded nucleic acid includes the barcodes of the barcode molecule on the 5′-end.

A template switching oligonucleotide (TSO) is an oligonucleotide that hybridizes to untemplated C nucleotides added by a reverse transcriptase during reverse transcription. The TSO can hybridize to the 3′ end of a cDNA molecule. The TSO can include one or more nucleotides with guanine (G) bases on the 3′-end of the TSO, with which the one or more cytosine (C) bases added by a reverse transcriptase to the 3′-end of a cDNA can hybridize. The series of G bases can comprise 1G base, 2 G bases, 3 G bases, 4 G bases, 5 G bases or more than 5 G bases. The series of G bases can be ribonucleotides. The reverse transcriptase can further extend the cDNA using the TSO as the template to generate a barcoded cDNA comprising the TSO.

The length of a TSO can be different in different embodiments. In some embodiments, a template switching oligonucleotide is, is about, is at least, is at least about, is at most, or is at most about, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values, nucleotides in length.

The number of the barcode molecules introduced into a partition comprising a TSO can be different in different embodiments. In some embodiments, the number of barcode molecules introduced into a partition comprising a TSO is, is about, is at least, is at least about, is at most, or is at most about, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 100000000, 20000000, 30000000, 40000000, 50000000, 60000000, 70000000, 80000000, 90000000, 10000000, 200000000, 300000000, 40000000, 500000000, 600000000, 700000000, 800000000, 90000000, 1000000000, or a number or a range between any two of these values.

In some embodiments, the TSO of the barcode molecules introduced into a partition can be identical. In some embodiments, the TSO of the barcode molecules introduced into a partition can be different. The percentage of the barcode molecules of the plurality of barcode molecules introduced into a partition with an identical TSO sequence can be different in different embodiments. In some embodiments, the percentage of the barcode molecules of the plurality of barcode molecules introduced into a partition with an identical TSO sequence is, is about, is at least, is at least about, is at most, is at most about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 100%, or a number or a range between any two of these values.

### Barcoding Target Nucleic Acids

The reaction described herein can comprise barcoding target nucleic acids associated with a cell in the partition (e.g., microwell) using the barcode molecules to generate barcoded nucleic acids (e.g., target nucleic acids each hybridized with a barcode molecule, single-stranded barcoded nucleic acids, or double-stranded barcoded nucleic acids).

Prior to barcoding the target nucleic acids, the reaction can comprise lysing cells (e.g. after introducing the barcode molecules and the cells to the partitions) to release the content of the cells within the partition. Lysis agents can be contacted with the cells or cell suspension concurrently, or immediately after the introduction of the cells into the partitions (e.g., microwells) and before the barcoding. In some embodiments, a lysis agent is introduced into (e.g., drawn into) the reaction chamber of the present microfluidic device as a reagent according to the process of using the device as described herein. Examples of lysis agents include bioactive reagents, such as lysis enzymes, or surfactant based lysis solutions including non-ionic surfactants such as TritonX-100 and Tween 20 and ionic surfactants such as sodium dodecyl sulfate (SDS). Lysis methods including, but not limited to, thermal, acoustic, electrical, or mechanical cellular disruption can also be used.

Synthesis of Single-Stranded Barcoded Nucleic Acids

In some embodiments, barcoding target nucleic acids associated with a cell in the partition can comprise extending the barcode molecules using the target nucleic acids as templates to generate partially single-stranded/partially double-stranded barcoded nucleic acids hybridized to the target nucleic acids in the partition (or after target nucleic acids hybridized with barcode molecules are pulled). The partially single-stranded/partially double-stranded barcoded nucleic acids hybridized to target nucleic acids can be separated by denaturation (e.g., heat denaturation or chemical denaturation using for example, sodium hydroxide) to generate single-stranded barcoded nucleic acids of the plurality of barcoded nucleic acids. The single-stranded barcoded nucleic acids can comprise a barcode molecule and an oligonucleotide complementary to the target nucleic acids. In some embodiments, the single-stranded barcoded nucleic acids can be generated by reverse transcription using a reverse transcriptase. For example, the single-stranded barcoded nucleic acids can be generated by using a DNA polymerase.

In some embodiments, the single-stranded barcoded nucleic acids can be cDNA produced by extending a barcode molecule using a target RNA associated with the cell as a template. The single-stranded barcoded nucleic acids can be further extended using a template switching oligonucleotide (TSO). A TSO is an oligo that hybridizes to untemplated C nucleotides added by a reverse transcriptase during reverse transcription. The TSO can be introduced into the partitions together with the reverse transcription reagents. For example, a reverse transcriptase can be used to generate a cDNA by extending a barcode molecule hybridized to an RNA. After extending the barcode molecule to the 5′-end of the RNA, the reverse transcriptase can add one or more nucleotides with cytosine (C) bases (e.g. two or three) to the 3′-end of the cDNA. The TSO can include one or more nucleotides with guanine (G) bases (e.g. two or more) on the 3′-end of the TSO. The nucleotides with G bases can be ribonucleotides. The G bases at the 3′-end of the TSO can hybridize to the cytosine bases at the 3′-end of the cDNA. The reverse transcriptase can further extend the cDNA using the TSO as the template to generate a cDNA with the reverse complement of the TSO sequence on its 3′-end. The barcoded nucleic acid can include the barcode sequences (e.g., cell barcode and UMI) on the 5′-end and a TSO sequence at its 3′-end.

In some embodiments, barcoding the target nucleic acids comprises extending the barcode molecules using the target nucleic acids as templates and the barcode molecules as TSO to generate single-stranded barcoded nucleic acids that are hybridized to the target nucleic acids.

In some embodiments, the barcode molecules are not attached to a particle and the barcode molecules can be TSO. For example, extension primers (e.g. oligonucleotides comprising a poly(dT) sequence) can be introduced into the partitions which hybridize to a target nucleic acid (e.g. the poly-adenylated mRNA). The extension primers can be extended using the target nucleic acids as a template. For example, a reverse transcriptase can be used to generate a cDNA by extending an extension primer hybridized to an RNA. After extending the extension primers to the 5′-end of the RNA, the reverse transcriptase can add one or more C bases (e.g. two or three) to the 3′-end of the cDNA. The TSO or barcode molecule can include one or more G bases (e.g. two or more) on the 3′-end of the TSO. The nucleotides with guanine bases can be ribonucleotides. The G bases at the 3′-end of the TSO or barcode molecule can hybridize to the cytosine bases at the 3′-end of the cDNA. The reverse transcriptase can switch template from the mRNA to the TSO or barcode molecule. The reverse transcriptase can further extend the cDNA using the TSO or barcode molecule as the template to generate a cDNA further comprising the reverse complement of the TSO or barcode molecule. In this case, the barcode sequences (e.g. cell barcode and molecular barcode) are on the 3′-end of the generated cDNA.

The single-stranded barcoded nucleic acids can be separated from the template target nucleic acids by digesting the template target nucleic acids (e.g., using RNase), by, chemical treatment (e.g., using sodium hydroxide), by hydrolyzing the template target nucleic acids, or via a denaturation or melting process by increasing the temperature, adding organic solvents, or increasing pH. Following the melting process, the target nucleic acids can be removed (e.g. washed away) and the single-stranded barcoded nucleic acids can be retained in the partition (e.g. through attachment to the partitions or through attachments to particles which can be retained in the partitions).

In some embodiments, the enzyme (e.g., DNA polymerase) and other agents (e.g., template switching oligonucleotide, extension primers, RNase, chemical treatment, or organic solvents) used for generating the single-stranded barcoded nucleic acids are introduced into the reaction chamber of the present microfluidic device as reagents according to the process of using the device as described herein.

Synthesis of Double-Stranded Barcoded Nucleic Acids

In some embodiments, barcoding target nucleic acids associated with the cell in the partition (e.g., microwell) can comprise generating barcoded nucleic acids comprising double-stranded barcoded nucleic acids in the partition using the single-stranded barcoded nucleic acids as templates (or after using the single-stranded barcoded nucleic acids are pooled). The double-stranded barcoded nucleic acids can be generated from the single-stranded barcoded nucleic acids retained in the partition using, for example, second-strand synthesis or one-cycle PCR.

The generated double-stranded barcoded nucleic acid can be denaturized or melted to generate two single-stranded barcoded nucleic acids: one single-stranded barcoded nucleic acid retained in the partition (e.g., attached to the particle) and the other single-stranded barcoded nucleic acid released into the solution from the retained single-stranded barcoded nucleic acid that can then be pooled to provide a pooled mixture outside the partitions. Both single-stranded barcoded nucleic acids (e.g. retained in the partitions or pooled outside the partitions) have a sequence comprising a sequence of a barcode molecule (e.g. a cell barcode sequence and/or a UMI barcode) and a sequence of a target nucleic acid or a reverse complement thereof.

In some embodiments, the enzyme and agents used for generating the double-stranded barcoded nucleic acids (e.g., those used in PCR reaction) are injected into the reaction chamber of the present microfluidic device as reagents according to the process of using the device as described herein. In some embodiments, following the barcoding reaction, the remaining reagents, solvents, and/or unwanted by-products are removed as waste liquid of the barcoding reaction from the reaction chamber into the waste liquid chamber of the present microfluidic device according to the process of using the device as described herein.

### Pooling

In some embodiments, the present method for nucleic acid analysis comprises pooling the barcoded nucleic acids after barcoding the target nucleic acids and before sequencing the barcoded nucleic acids to obtain pooled barcoded nucleic acids. In some embodiments, the barcoded nucleic acids are pooled or collected as products of the barcoding reaction from the reaction chamber into the product chamber of the present microfluidic device according to the process of using the device as described herein. In some embodiments, synthesis of single-stranded barcoded nucleic acids and double-stranded barcoded nucleic acids occur after the pooling of target nucleic acids hybridized with barcode molecules.

In some embodiments, the barcode molecules are attached to particles, only single-stranded barcoded nucleic acids released into bulk are collected by pooling, and the particles are not pooled (e.g. not removed from the partitions) but retained in the partitions (e.g. by an external magnetic field applied on magnetic beads), thereby allowing one to trace the origin of the pooled barcoded nucleic acids, for example, to its original location in the partitions.

The pooled barcoded nucleic acids can be single-stranded or double-stranded (e.g. generated from the single-stranded pooled barcoded nucleic acids by PCR amplification). The pooled barcoded nucleic acids (e.g. barcoded cDNA) can be purified and/or amplified prior to sequencing library construction. The pooled barcoded nucleic acids with desired length may be selected.

### Sequencing Library Construction

The barcoded nucleic acids (e.g. pooled barcoded nucleic acids) are further processed prior to sequencing to generate processed barcoded nucleic acids. In some embodiments, the barcoded nucleic acids are pooled or collected as a product from the reaction chamber of the present microfluidic device, and the further processing of the barcoded nucleic acids are conducted under appropriate conditions. For example, the method herein can include amplification of barcoded nucleic acids, fragmentation of amplified barcoded nucleic acids, end repair of fragmented barcoded nucleic acids, A-tailing of fragmented barcoded nucleic acids that have been end-repaired (e.g., to facilitate ligation to adapters), and attaching (e.g. by ligation and/or PCR) with a second sequencing primer sequence (e.g. a Read 2 sequence), sample indexes (e.g. short sequences specific to a given sample library), and/or flow cell binding sequences (e.g. P5 and/or P7). Additional PCR amplification can also be performed. This process can also be referred to as sequencing library construction.

In some embodiments, the method comprises performing a polymerase chain reaction in bulk, subsequent to the pooling, on the pooled barcoded nucleic acids, thereby generating amplified barcoded nucleic acids. PCR amplification can be carried out to generate sufficient mass for the subsequent library construction processes. PCR amplification can also be performed with primers specific to target nucleic acids of interest such as T-cell receptor (TCR) or B-cell receptor (BCR) constant regions.

In some embodiments, the method comprises fragmenting (e.g., via enzymatic fragmentation, mechanical force, chemical treatment, etc.) the pooled barcoded nucleic acids to generate fragmented barcoded nucleic acids. Fragmentation can be carried out by any suitable process such as physical fragmentation, enzymatic fragmentation, or a combination of both. For example, the barcoded nucleic acids can be sheared physically using acoustics, nebulization, centrifugal force, needles, or hydrodynamics. The barcoded nucleic acids can also be fragmented using enzymes, such as restriction enzymes and endonucleases.

Fragmentation yields fragments of a desired size for subsequent sequencing. The desired sizes of the fragmented nucleic acids are determined by the limitations of the next generation sequencing instrumentation and by the specific sequencing application as will be understood by a person skilled in the art. For example, when using Illumina technology, the fragmented nucleic acids can have a length of between about 50 bases to about 1,500 bases. In some embodiments, the fragmented barcoded nucleic acids have about 100 bp to 700 bp in length.

Fragmented barcoded nucleic acids can undergo end-repair and A-tailing (to add one or more adenine bases) to form an A overhang. This A overhang allows adapter containing one or more thymine overhanging bases to base pair with the fragmented barcoded nucleic acids.

Fragmented barcoded nucleic acids can be further processed by adding additional sequences (e.g. adapters) for use in sequencing based on specific sequencing platforms. Adapters can be attached to the fragmented barcoded nucleic acids by ligation using a ligase and/or PCR. For example, fragmented barcoded nucleic acids can be processed by adding a second sequencing primer sequence. The second sequencing primer sequence can comprise a Read 2 sequence. An adapter comprising the second primer sequence can be ligated to the fragmented barcoded nucleic acids after, for example, end-repair and A tailing, using a ligase. The adaptor can include one or more thymine (T) bases that can hybridize to the one or more A bases added by A tailing. An adaptor can be, for example, partially double-stranded or double stranded.

The adapter can also include platform-specific sequences for fragment recognition by specific sequencing instrument. For example, the adapter can comprise a sequence for attaching the fragmented barcoded nucleic acids to a flow well of Illumina platforms, such as a P5 sequence, a P7 sequence, or a portion thereof. Different adapter sequences can be used for different next generation sequencing instrument as will be understood by a person skilled in the art.

The adapter can also contain sample indexes to identify samples and to permit multiplexing. Sample indexes enable multiple samples to be sequenced together (i.e. multiplexed) on the same instrument flow cell as will be understood by a person skilled in the art. Adapters can comprise a single sample index or a dual sample indexes depending on the implementations such as the number of libraries combined and the level of accuracy desired.

In some embodiments, the amplified barcoded nucleic acids generated from sequencing library construction can include a P5 sequence, a sample index, a Read 1 sequence, a cell barcode, a UMI, a poly(dT) sequence, a target biding region, a sequence of a target nucleic acid or a portion thereof, a Read 2 sequence, a sample index, and/or a P7 sequence (e.g., from 5′-end to 3′-end). In some embodiments, the amplified barcoded nucleic acids can include a P5 sequence, a sample index, a Read 1 sequence, a cell barcode, a UMI, a sequence of a template switching oligonucleotide, a sequence of a target nucleic acid or a portion thereof, a Read 2 sequence, a sample index, and/or a P7 sequence (e.g., from 5′-end to 3′-end).

In some embodiments, sequencing the barcoded nucleic acids, or products thereof, comprises sequencing products of the barcoded nucleic acids. Products of the barcoded nucleic acids can include the processed nucleic acids generated by any step of the sequencing library construction process, such as amplified barcoded nucleic acids, fragmented barcoded nucleic acids, fragmented barcoded nucleic acids comprising additional sequences such as the second sequencing primer sequence and/or adapter sequences described herein.

### Sequencing Barcoded Nucleic Acids

The method disclosed herein can comprise sequencing the barcoded nucleic acids or products thereof to obtain nucleic acid sequences of the barcoded nucleic acids. The barcoded nucleic acids generated by the method disclosed herein comprise barcoded nucleic acids retained in a partition and barcoded nucleic acids pooled, from each partition, into a pooled mixture outside the partitions. The barcoded nucleic acids retained in a partition and the pooled barcoded nucleic acids in a pooled mixture outside the partitions can be sequenced using a same or different sequencing techniques.

### Sequencing Pooled Barcoded Nucleic Acids

In some embodiments, sequencing the plurality of barcoded nucleic acids or products thereof comprises sequencing the pooled barcoded nucleic acids to obtain nucleic acid sequences of the pooled barcoded nucleic acids. As used herein, a “sequence” can refer to the sequence, a complementary sequence thereof (e.g., a reverse, a compliment, or a reverse complement), the full-length sequence, a subsequence, or a combination thereof. The nucleic acids sequences of the pooled barcoded nucleic acids can each comprise a sequence of a barcode molecule (e.g. the cell barcode and the UMI) and a sequence of a target nucleic acid associated with the cell or a reverse complement thereof.

Pooled barcoded nucleic acids can be sequenced using any suitable sequencing method identifiable to a person skilled in the art. For example, sequencing the pooled barcoded nucleic acids can be performed using high-throughput sequencing, pyrosequencing, sequencing-by-synthesis, single-molecule sequencing, nanopore sequencing, sequencing-by-ligation, sequencing-by-hybridization, next generation sequencing, massively-parallel sequencing, primer walking, and any other sequencing methods known in the art and suitable for sequencing the barcoded nucleic acids generated using the methods herein described.

Post-Sequencing Analysis

The obtained nucleic acid sequences of the plurality of barcoded nucleic acids (e.g. nucleic acid sequences of pooled barcoded nucleic acids) can be subjected to any downstream post-sequencing data analysis as will be understood by a person skilled in the art. The sequence data can undergo a quality control process to remove adapter sequences, low-quality reads, uncalled bases, and/or to filter out contaminants. The high-quality data obtained from the quality control can be mapped or aligned to a reference genome or assembled de novo.

Gene expression quantification and differential expression analysis can be carried out to identify genes whose expression differs in different cells. Barcoded target nucleic acids from a cell can have an identical cell barcode in the sequencing data and can be identified. Barcoded target nucleic acids from different cells can have different cell barcodes in the sequencing data and can be identified. Barcoded target nucleic acids with an identical cell barcode, an identical target sequence, and different molecular barcodes in the sequencing data can be quantified and used to determine the expression of the target.

In some embodiments, the method can comprise determining a profile (e.g. an expression profile, an omics profile, or a multi-omics profile) of the target nucleic acids associated with the cell. A profile can be a single omics profile, such as a transcriptome profile. The profile can be a multi-omics profile, which can include profiles of genome (e.g. a genomics profile), proteome (e.g. a proteomics profile), transcriptome (e.g. a transcriptomics profile), epigenome (e.g. an epigenomics profile), metabolome (e.g. a metabolomics profile), and/or microbiome (e.g. microbiome profile). The profile can include an RNA expression profile. The profile can include a protein expression profile. The expression profile can comprise an RNA expression profile, an mRNA expression profile, and/or a protein expression profile. The profile can be a profile of a single cell (e.g., under normal conditions and/or in response to an external stimuli). The profiles can be profiles of two cells from a same partition (e.g. a first cell and a second cell in a cell-cell interaction analysis). A profile can also be a profile of one or more target nucleic acids (e.g. gene markers) or a selection of genes associated with the single cell (or the first cell and/or the second cell in a cell-cell interaction analysis).

In some embodiments, the method disclosed herein can be used to determine a profile (e.g., an expression profile, an omics profile, or a multi-omics profile) of a cell under an external stimuli and/or involved in cell-cell interaction, such as to detect changes in gene expression profile of the cell in terms of identification of RNA transcripts and their quantitation. In some embodiments, a profile of a first cell and/or a second cell can be determined using the nucleic acid sequences of the barcoded nucleic acids. For example, determining the profile of the first cell and/or the second cell can comprise determining the profile of the first cell and/or the second cell using the second barcode sequences (e.g. UMI) and sequences of the target nucleic acids, or a portion thereof, present in the nucleic acid sequences.

In some embodiments, the first cell and the second cell when in contact (or under incubation) with each other can have an expression profile different from an expression profile of the first cell or the second cell alone. A differential expression analysis can be performed to detect quantitative changes in expression levels between the cell involved in a cell-cell interaction and the cell alone. Genes expressed differentially can be detected. Differential expression profile can be correlated to the cell functionality and/or cell's phenotypes.

Therefore, in some embodiments, an interaction between the first cell and the second cell may be of interest. The interaction between the first cell and the second cell that is of interest can comprise a change in a profile of the first cell and/or the second cell. The profile can comprise a transcriptomics profile, a multi-omics profile such as a genomics profile, a proteomics profile, a transcriptomics profile, an epigenomics profile, a metabolomics profile, a chromatics profile, or a combination thereof. In some embodiments, the profile of the first cell and/or the second cell in the partition can be different from a profile of the first cell or the second cell alone.

### EXAMPLES

Some aspects of the embodiments discussed above are disclosed in further detail in the following examples, which are not in any way intended to limit the scope of the present disclosure.

### Example 1

### Microfluidic Device

A microfluidic chip capable of performing reagent exchange is provided. The microfluidic chip is an integral disposable chip including a reagent exchange unit and a working unit (also described as a reaction unit herein) bonded to each other.

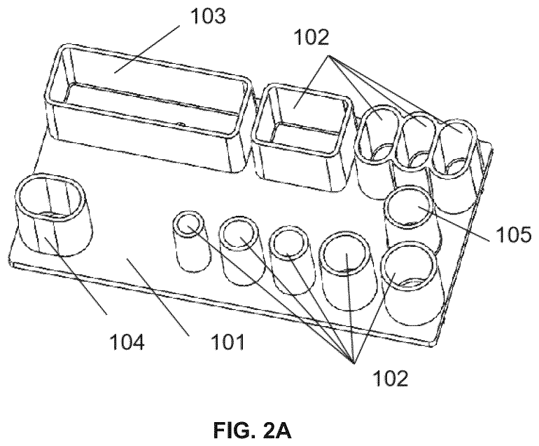

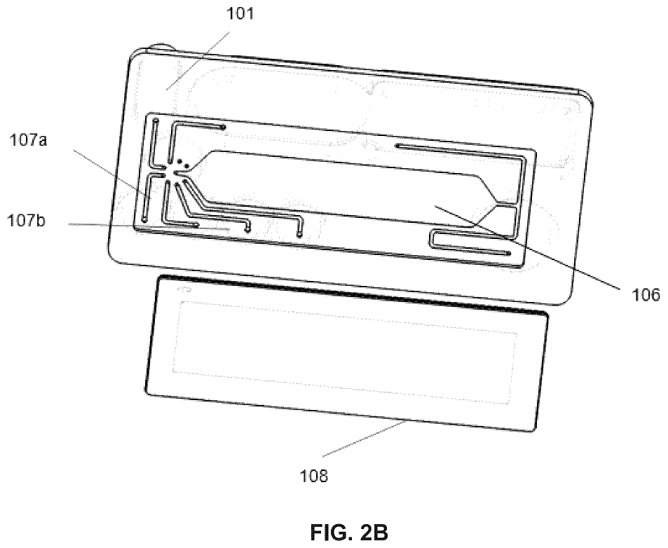

A representative structure of the reagent exchange unit (such as a reagent exchange cartridge) of the microfluidic chip is shown in FIGS. 2A (top view) and 2B (bottom view).

A left portion of an upper surface of the reagent exchange unit 101 includes a circular product reservoir 104 and a rectangular waste reservoir 103; and a right portion includes one reagent exchange reservoir 105 and six reagent reservoirs 102, where four of them are circles of the same size, and the remaining two have different sizes but the same shape each being an oval shape (with semicircles on two ends and a rectangle in the middle).

The upper surface of the reagent exchange unit 101 may be also viewed as including an upper side and a lower side, the rectangular waste liquid reservoir 103 and the two reagent reservoirs 102 of different sizes but the same shape are located on the upper side, and the circular product reservoir 104, the four reagent reservoirs 102 of the same size, and the reagent exchange reservoir 105 are located on the lower side.

The microfluidic chip further includes a rectangular working unit 108 bonded to the lower surface of the reagent exchange unit. A working section 106 (also referred to as a reaction chamber herein) is formed between the working unit and the reagent exchange unit after the two units are bonded to each other. The working section 106 is used for mixing reagents and performing the reaction.

The reagent reservoirs 102 are all connected to the reagent exchange reservoir 105 through microchannels 107. The microchannels 107 include reagent channels 107a to transfer a reagent. The microchannel (such as 107b) may allow for homogeneous mixing of liquids. The reagent exchange reservoir 105 has an exit opening, which is connected to the working section 106. In the reagent exchange reservoir, the openings of the microchannels from the reagent reservoir may be set above the exit opening connected to the working section. The design of the reagent exchange reservoir 105 avoids cross-contamination of different reagents.

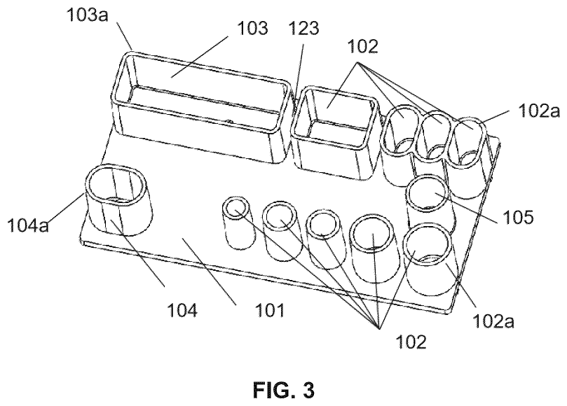

Another non-limiting example of a reagent exchange cartridge is provided, as shown in FIG. 3. A left portion of the reagent exchange cartridge includes a product reservoir 104 in a shape with semicircles on two ends and a rectangle in the middle and a rectangular waste liquid reservoir 103; and a right portion of the reagent exchange cartridge includes one circular reagent exchange reservoir 105 and nine reagent reservoirs 102. Five of the reagent reservoirs are on the lower side, in circular shape, and of same size. Three of the reagent reservoirs are on the upper side, in an oval shape, and of same size. The remaining reagent reservoir is of rectangular shape, and is on the upper side between the waste liquid reservoir 103 and the other reagent reservoirs.

The reagent reservoirs and the waste reservoir on the upper side are joined by shared wall 123. The reagent reservoirs on the upper side, and the reagent reservoirs on the lower side may be joined by shared walls 122. The reagent exchange reservoir 105 is joined with the upper and lower reagent reservoirs by shared wall 125. The reagent reservoirs, the waste reservoir, and the product reservoir may have additional structural features, such as protrusions 102a, 103a, 104a on the outside.

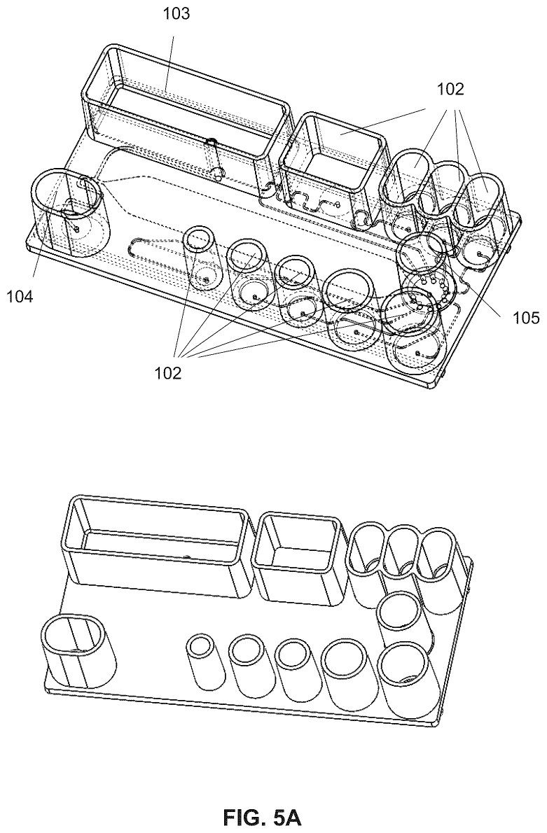

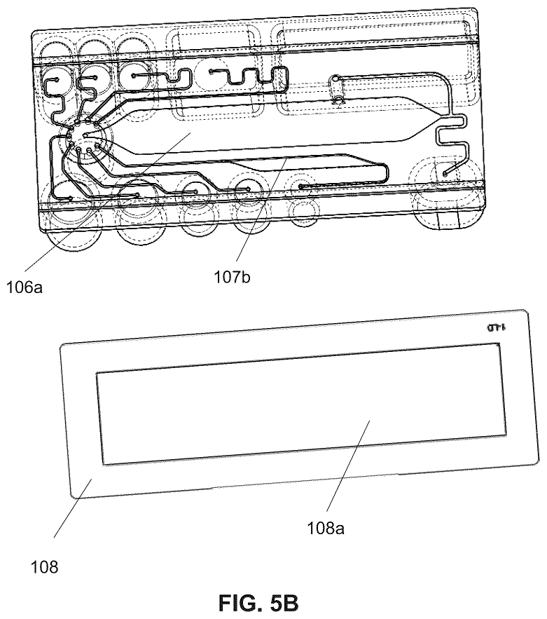

Yet another non-limiting example of a reagent exchange cartridge is provided, as shown in FIGS. 5A and 5B. Similar to the structure of FIG. 3, this reagent exchange cartridge has nine reagent reservoirs 102. However, the five circular reagent reservoirs on the lower side have different sizes. Further, all the reservoirs are separate structures which are not joined by shared walls, except for three reagent reservoirs on the upper side. The working section 106 provide a space 106a for different reagents to flow through, thereby achieving reagent exchange. The working unit 108 provides an surface 108a to perform a reaction. The surface 108a can comprise, for example, a microwell array.

### Example 2

### Use of the Microfluidic Device

A representative method of using the microfluidic chips according to Example 1 is provided. The method includes the following steps:
(1) The reagent exchange unit 101 and the working unit 108 are bonded, and reagents needed for the reaction are placed in corresponding reservoirs of the reagent exchange unit 101. For the device in FIGS. 2A and 2B, a total of six different reaction reagents can be placed on the microfluidic chip, and corresponding reagent reservoirs 102 can be selected according to volumes of the reaction reagents.
(2) A reagent is pressurized so that the reagent flows to the reagent exchange reservoir 105 along a microchannel 107 on the bottom of the reagent exchange unit 101, and then the waste reservoir 103 or the product reservoir 104 is depressurized so that the reaction reagents in the reagent exchange reservoir 105 flow into the working section 106.
(3) The above steps are repeated according to the type of the reagent needed, so as to complete the depressurization and pressurization process of different reagents, and a continuous reagent exchange process is performed on the microfluidic chip until the reaction is completed.

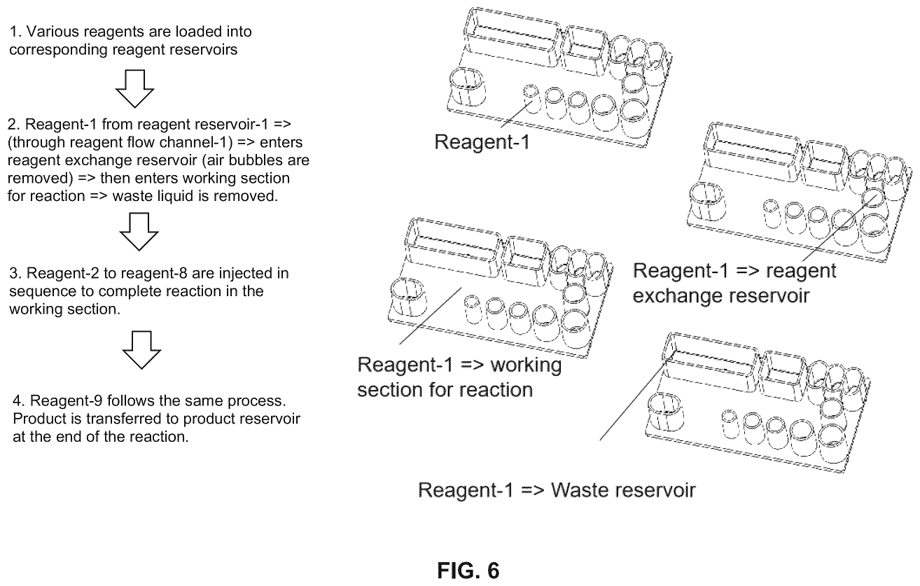

The method can also be carried out using the device of FIG. 3 or FIGS. 5A-5B, each of which allows for loading and exchanging nine reagents in a reaction. As illustrated in FIG. 4 and FIG. 6, the reagents are loaded in corresponding reagent reservoirs. The first reagent (Reagent-1) is injected to the reagent exchange chamber through reagent flow channel-1. Advantageously, air bubbles in the reagent are removed. The remaining reagents are injected in sequence and the reaction is allowed to proceed to completion. Unwanted liquid from the reaction is removed to the waste reservoir. The product from the reaction is then isolated by transferring to the product reservoir.

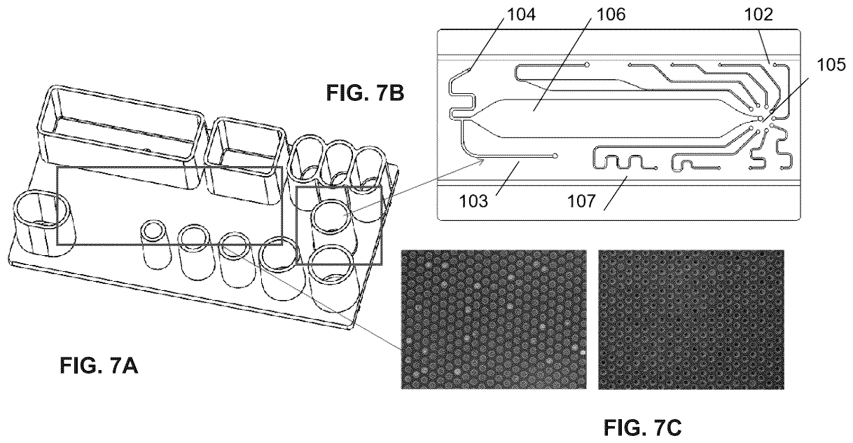

FIGS. 7A-7C further illustrate the reagent exchange process and the reaction using a device similar to the one in FIG. 3. The microchannels 107 connecting the reagent reservoirs 102 and the reagent exchange reservoir 105 are indicated by colored liquid (FIG. 7B). The reagent exchange reservoir 105 is in fluid communication with an inlet of the working section 106 (filled with colored liquid), and allows for sequential injection of different reagents. An outlet of the working section 106 is connected with the waste reservoir 103 and the product reservoir 104 by microchannels.

### Example 3

### Single Cell Sample Preparation

A single-cell sample preparation experiment is conducted using the microfluidic chip provided in Example 1. The specific steps are as follows:

First, corresponding needed reaction reagents, including a cell sample, a rinsing solution, a lysis solution, and molecular markers, are placed in various reagent reservoirs 102.

Afterwards, the cell sample enters the working section 106, and the working unit 108 captures single cells, and then the rinsing solution enters the working section 106 to rinse out unwanted impurity molecules.

Subsequently, the molecular markers (such as beads attached with barcoding molecules) enter and mark each single cell.

After the lysis solution enters the working section 106, nucleic acids in the cells are extracted, and finally the product is transferred into the product reservoir 104 to complete the operation.

As a non-limiting example, the working section 106 may include a microwell array as disclosed herein (e.g., on the surface 108a of the working unit 108). The cells from a reagent reservoir are injected into the working section, and are partitioned across the microwells of the microwell array, as illustrated in FIG. 7C (left panel). Similarly, beads attached with barcoding molecules may be injected into the working section 106 as a separate reagent, and are partitioned across the microwells of the microwell array (FIG. 7C, right panel).

It should be noted that in the present disclosure, as long as reactions involving exchange of different reagents are involved, automatic reagent exchange can be accomplished using this design idea. Therefore, the number, shapes, volumes, and the like of the reservoirs of the microfluidic chip are not limited. For example, reagents such as magnetic beads for library construction, a PBS buffer, and absolute ethanol are often needed in the library establishment process, and a reagent is also needed to repeatedly rinse the product, so that automatic operation can be performed according to this design principle; meanwhile, if the process is to be integrated or the throughput is to be increased, the same effect can also be achieved by increasing the number and volume of the reagent reservoirs of the microfluidic chip.

In view of the above, the microfluidic chip capable of performing reagent exchange as described herein has wide application scenarios, the reagent exchange unit and the working unit are combined, and due to the design of the reagent exchange reservoir and the microchannels, automatic exchange of reagents is realized on the chip, so that the accuracy of different reagents during exchange is ensured, waste of reagents is avoided, and the need for manpower and machinery is reduced; meanwhile, an integral disposable microfluidic chip is manufactured according to needs, so that cross-contamination can be effectively avoided.

### Example 4

### Cell Reaction

A specific method for performing a cell reaction is provided as follows, with reference to FIGS. 8A, 8B, 9A, 9B:
(1) injecting various reaction reagents into various reagent reservoirs 202 in the cell reaction plate 201 in advance, including nine reagents of a cell buffer, a chip surface treatment solvent, a cell lysis solution, a reverse transcription reagent, a reverse transcription cleaning solution, and tag magnetic beads;
(2) feeding a driving gas into the driving gas path channels 210 through the solenoid valves 208, injecting the driving gas into corresponding reagent reservoirs 202 along the independent driving gas path channels 210, and pressing the reaction reagents stored in the reagent reservoirs 202 into the buffer reservoir 203 under the pressure of the driving gas;
(3) performing gas extraction on the waste reservoir 204 through the waste liquid gas extraction channel, so that the reaction reagents in the buffer reservoir 203 are drawn into the reaction chamber to complete reagent injection;
(4) repeatedly performing step (2) and step (3), so that the reaction reagents in the reagent reservoirs 202 are all injected into the reaction chamber 206, controlling the temperature for cell reaction to 42° C. and reacting for 1.5 h to obtain a DNA product; and
(5) after the cell reaction ends, performing gas extraction on the waste reservoir 204 through the waste liquid gas extraction channel again, so that a waste in the reaction chamber 206 enters the waste reservoir 204; performing gas extraction on the product reservoir 205 through the product gas extraction channel, so that a reaction product in the reaction chamber 206 enters the product reservoir 205.

### Example 5

### Cell Sample Preparation

A preparation method for preparing a cell sample by using the sample device as described herein is provided. The preparation method particularly includes the following steps, with references to FIGS. 11, 12A-12B, and 13:
(I) injecting reagents, including a cell buffer, a chip surface treatment solvent, a cell lysis buffer, a reverse transcription reagent, a reverse transcription cleaning solution, magnetic label beads and the like, respectively into a plurality of reagent chambers, placing the reaction chip between the gas-path control board 302 and the heating plate 301, activating, under the control of the control unit 304, the plurality of gas injection solenoid valves 313 and pressing the reagents respectively into the buffer chamber by injecting gas into the plurality of reagent chambers, and activating, under the control of the control unit 304, the gas extraction solenoid valve 311 of the waste reservoir 307 and extracting gas from the waste reservoir 307 to draw the reaction reagents in the buffer reservoir 314 into the reaction chamber 308; and
(II) controlling, using the control unit 304, the heating plate 301 to heat the reaction chamber 308 to perform reverse transcription at a temperature of 42° C., and obtaining the cell sample; and activating, under the control of the control unit 304, the gas extraction solenoid valve 311 of the product reservoir 306, and drawing the cell sample in the reaction chamber 308 into the product reservoir 306.

The time for the entire sample preparation process was 1.5 h.

Via the arrangement of the reagent reservoirs 305 on the reaction chip, in combination with injection of gas into the reagent reservoirs 305 via the driving gas-path channel 312 on the gas-path control board 302, the injection of the reaction reagents into the reaction chamber 308 to perform cell reaction is thereby achieved. The reaction reagents are injected into the buffer reservoir 314 under the control of the driving gas, to achieve the injection of different reagents in batches or together, thereby achieving the quantitative injection of different reaction reagents, which effectively reduces the operation difficulty for an operator. The matching of structures of the gas-path control board 302 and the reaction chip simplifies the structure of the reaction unit. Further, via the arrangement of the heating plate 301 to heat the reaction chip, the present sample preparation device is capable of performing reverse transcription, and the present cell preparation device has benefits such as a simple structure, easy operation, a small footprint and high adaptability.

### Example 6

### Automated Cell Reaction System

A method for carrying out a cell reaction using the reaction system as described herein is provided. The method is suitable for preparing a cell sample, such as a single cell sample. The method includes, with references to FIGS. 14A-14C, 15A-15B, 16, and 17A-17B:
(I) injecting different reaction reagents into the respective reagent reservoirs 511 in advance, including nine reagents of a cell buffer, a chip surface treatment solvent, a cell lysate, a reverse transcription reagent, a reverse transcription cleaning solution and tag magnetic beads; driving, by the horizontal movement module, the reaction platform 520 to move away from directly below the integrated gas-path control module 400; fixing, on the reaction platform 520, the cell reaction plate 510 into which the reaction reagents have been injected; then driving the reaction platform 520 to return to the original position by means of the horizontal movement module; and driving, by the vertical movement module, the gas-path platform to press downward such that the integrated gas-path control board 410 is attached to the cell reaction plate 510;
(II) injecting the driving gas into the driving gas channels 413 via a solenoid valve 411; injecting the driving gas into the reagent reservoirs 511 along the independent driving gas channels 413 corresponding thereto; driving the reaction reagents stored in the reagent reservoirs 511 into the buffer reservoir 512 by means of the pressure of the driving gas; and extracting gas from the waste reservoir 513 through a waste liquid gas extraction channel, such that the reaction reagents in the buffer reservoir 512 are drawn into the reaction chamber to complete reagent injection; and heating each cell reaction plate 510 by the heating element and controlling the temperature of the cell reaction to be at 42° C., so as to obtain a DNA product after 1.5 h of reaction, and
(III) after the cell reaction is complete, extracting gas from the waste reservoir 513 through the waste gas extraction channel again, such that a waste in the reaction chamber 515 is drawn into the waste liquid reservoir 513; and extracting gas from the product reservoir 514 through a product gas extraction channel, such that a reaction product in the reaction chamber 515 is drawn into the product reservoir 514.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a.” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A. B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B. and C together, etc.). In those instances where a convention analogous to “at least one of A, B. or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

The numerical range in the present application not only includes the point values listed above, but also includes any point values in the above numerical ranges that are not listed. Due to space limitations and for the sake of simplicity, the present invention will not exhaustively list the specific point values included in the range.

It should be understood that in the description of the present application, terms such as “center”, “longitudinal”, “lateral”, “up”, “down”, “front”, “back”, “left”, “right”, “vertical”, “horizontal”, “top”, “bottom”, “inside”, and “outside” for indicating orientations or positional relationships refer to orientations or positional relationships as shown in the drawings; the terms are for the purpose of describing the present application and simplifying the description rather than indicating or implying that the device or element must have a certain orientation and be structured or operated by the certain orientation, and therefore cannot be regarded as limitation to the present application.

It should be noted that in the description of the present application, unless otherwise expressly specified and defined, the terms “arrange,” “disposed,” “connected,” “connecting,” or alike, should be understood in a broad sense. For example, the connection may be fixed connection, detachable connection, or integrated connection; may be mechanical connection or electrical connection; may be direct connection or indirect connection through an intermediate medium or internal communication between two elements. Those of ordinary skill in the art can understand the specific meaning of the aforementioned terms in the present application according to the specific situation.

Furthermore, the terms “first,” “second,” and so on are used for descriptive purposes only and are not to be construed as indicating or implying relative importance or implicitly indicating the number of indicated technical features. Thus, the features defined with “first,” “second,” and so on may explicitly or implicitly include one or more of the features. In the description of the present invention, unless otherwise indicated, “multiple” or “a plurality of” means two or more.

It should be understood by those skilled in the art that the present application necessarily includes necessary pipelines, conventional valves, and general-purpose pump equipment for achieving process completeness. However, the above content does not belong to main inventive points of the present application. Those skilled in the art can make their own layout based on process flow and equipment structure selection, which is not specially required or specifically defined in the present application.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A microfluidic device comprising:
a reagent exchange unit comprising a plurality of reagent reservoirs and a reagent exchange reservoir on an upper surface of the reagent exchange unit;
a reaction unit;
a reaction chamber and a plurality of fluid microchannels formed between a lower surface of the reagent exchange unit and a upper surface of reaction unit, wherein the reaction chamber comprises an inlet and an outlet, wherein fluid microchannels of the plurality of fluid microchannels connect (i) reagent reservoirs of the plurality of reagent reservoirs and (ii) the reagent exchange reservoir, and wherein the reagent exchange reservoir is connected with the inlet of the reaction chamber.

2. A microfluidic device comprising:
a reagent exchange unit comprising a plurality of reagent reservoirs and at least one reagent exchange reservoir;
a reaction unit; and
a reaction chamber and a plurality of fluid microchannels formed between a surface of the reagent exchange unit and a surface of reaction unit, wherein each of fluid microchannels of the plurality of fluid microchannels connects (i) a reagent reservoir of the plurality of reagent reservoirs and (ii) the reagent exchange reservoir, and wherein the reagent exchange reservoir is connected with an inlet of the reaction chamber.

3. A microfluidic device comprising:
a plurality of reagent reservoirs and at least one reagent exchange reservoir;
a reaction chamber; and
a plurality of fluid microchannels, wherein each of fluid microchannels of the plurality of fluid microchannels connects (i) a reagent reservoir of the plurality of reagent reservoirs and (ii) the reagent exchange reservoir, and wherein the reagent exchange reservoir is connected with an inlet of the reaction chamber.

4. A microfluidic device comprising:
a plurality of reagent reservoirs and at least one reagent exchange reservoir;
a reaction chamber; and
a plurality of fluid microchannels, wherein different fluid microchannels of the plurality of fluid microchannels connect (i) different reagent reservoirs of the plurality of reagent reservoirs and (ii) the reagent exchange reservoir, and wherein the reagent exchange reservoir is in fluid communication with the reaction chamber.

5. A microfluidic device comprising:
a plurality of reservoirs;
a reaction chamber; and
a plurality of fluid microchannels, wherein each of the plurality of reservoirs is connected with at least one other reservoir of the plurality of reservoirs via a fluid microchannel of the plurality of fluid microchannels, wherein at least one reservoir of the plurality of reservoirs is connected with at least two other reservoirs of the plurality of reservoirs, and wherein the at least one reservoir is in fluid communication with the reaction chamber.

6. A microfluidic device comprising:
a plurality of reservoirs;
a reaction chamber; and
a plurality of fluid microchannels, wherein one, one or more, or each of the plurality of reservoirs is connected with at least one other reservoir of the plurality of reservoirs via a fluid microchannel of the plurality of fluid microchannels and/or the reaction chamber, optionally via a fluid microchannel of the plurality of fluid microchannels, optionally wherein at least one reservoir of the plurality of reservoirs is connected with at least two other reservoirs of the plurality of reservoirs.

7. The microfluidic device of any one of claims 3-6, wherein the microfluidic device comprises a first layer and a second layer reversibly coupled to each other, wherein the first layer comprises a plurality of grooves, wherein the second layer covers the plurality of grooves to form the plurality of fluid microchannels, wherein the first layer comprises a cavity, and/or wherein the second layer covers the cavity to form the reaction chamber.

8. A microfluidic device comprising a reagent exchange unit and a reaction unit bonded to each other,
wherein a first surface of the reagent exchange unit comprises a plurality of reagent reservoirs, a product reservoir, a waste reservoir, and a reagent exchange reservoir, and wherein all reagent reservoirs of the plurality of reagent reservoirs, the product reservoir, and the waste reservoir are connected to the reagent exchange reservoir through a plurality of fluid microchannels and/or a reaction chamber on a second surface of the reagent exchange unit, and
wherein the reaction unit covers the plurality of microchannels and the reaction chamber, and forms, together with the second surface of the reagent exchange unit, the plurality of microchannels and the reaction chamber of the microfluidic device.

9. A microfluidic device comprising a reagent exchange unit and a reaction unit bonded to each other,
wherein an upper surface of the reagent exchange unit comprises a plurality of reagent reservoirs, a product reservoir, a waste reservoir, and a reagent exchange reservoir, and wherein all reagent reservoirs of the plurality of reagent reservoirs, the product reservoir, and the waste reservoir are connected to the reagent exchange reservoir through a plurality of fluid microchannels and/or a reaction chamber on a lower surface of the reagent exchange unit and in a recess of the lower surface of the reagent exchange unit,
wherein the recess is connected to the reagent exchange reservoir, the product reservoir, and the waste reservoir, and
wherein the reaction unit covers the plurality of microchannels, the reaction chamber, and the recess, and forms, together with the recess and the lower surface of the reagent exchange unit, the plurality of microchannels and the reaction chamber of the microfluidic device.

10. The microfluidic device of any one of claim 1-9, wherein the reagent exchange unit is in direct contact with the reaction unit, wherein the reagent exchange unit and the reaction unit are bonded to each other, and/or wherein the reagent exchange unit and the reaction unit form an integral structure.

11. The microfluidic device of any one of claims 1-10, wherein the reagent exchange unit further comprises a waste reservoir on the upper surface of the reagent exchange unit, wherein a waste fluid microchannel of the plurality of fluid microchannels connects the waste reservoir and the outlet of the reaction chamber, optionally wherein the waste fluid microchannel directly connects the waste reservoir and the outlet of the reaction chamber.

12. The microfluidic device of any one of claims 1-11, wherein the reagent exchange unit further comprises a product reservoir on the upper surface of the reagent exchange unit, wherein a product fluid microchannel of the plurality of fluid microchannels connects the product reservoir and the outlet of the reaction chamber, optionally wherein the product fluid microchannel directly connects the product reservoir and the outlet of the reaction chamber.

13. The microfluidic device of claim 12, wherein the waste fluid microchannel, the product fluid microchannel, and the outlet of the reaction chamber connect at a junction, or wherein the waste fluid microchannel and the product fluid microchannel merge into a single fluid microchannel which is connected to the outlet of the reaction chamber.

14. The microfluidic device of any one of claims 1-13,
wherein the plurality of reagent reservoirs comprises a mixing reservoir,
wherein a mixing fluid microchannel of the plurality of fluid microchannels connects the mixing reservoir and the reagent exchange reservoir, optionally wherein the mixing fluid microchannel splits into two or more fluid microchannels which merge into a single fluid microchannel, and optionally wherein a first portion of the mixing fluid microchannel connects the mixing reservoir and a mixing chamber and a second portion of the mixing fluid microchannel connects the mixing chamber and the reagent exchange reservoir.

15. The microfluidic device of any one of claims 1-14, wherein one or more reagent reservoirs of the plurality of reagent reservoirs, the waste reservoir, and/or the product reservoir each comprises an opening that connects the reservoir to a fluid microchannel of the plurality of fluid microchannels, wherein the reagent exchange reservoir comprises one or more openings that connect the reagent exchange reservoir to one or more fluid microchannels of the plurality of fluid microchannels, and/or wherein the reagent exchange reservoir comprises an opening that connects the reagent exchange reservoir to the inlet of the reaction chamber.

16. The microfluidic device of any one of claims 1-15, wherein one or more reagent reservoirs of the plurality of reagent reservoirs, the reagent exchange reservoir, the waste reservoir, and/or the product reservoir each is formed by a wall protruding from the upper surface of the reagent exchange unit and/or each comprises a tapered lower surface and/or a rounded lower surface, optionally wherein the tapered lower surface and/or the rounded lower surface, or a portion thereof, is disposed in or protrudes into the upper surface of the reagent exchange unit.

17. The microfluidic device of any one of claims 1-16, wherein the plurality of reagent reservoirs comprises at least two reagent reservoirs, wherein fluid microchannels of the plurality of fluid microchannels connecting reagent reservoirs of the plurality of reagent reservoirs to the reagent exchange reservoirs comprise at least two fluid microchannels, and/or wherein the number of the reagent reservoirs and the number of the fluidic microchannels connecting the reagent reservoirs to the reagent exchange reservoir are identical.

18. The microfluidic chip of any one of claims 1-17, wherein the upper surface of the reagent exchange unit is divided into a first functional area and a second functional area, wherein the first functional area comprises the product reservoir and the waste reservoir, and wherein the second functional area comprises at least two reagent reservoirs, optionally wherein the second functional area comprises the reagent exchange reservoir.

19. The microfluidic chip of any one of claims 1-18, wherein one, one or more, or each of the plurality of reagent reservoirs comprises a reagent, optionally wherein two of the plurality of reagent reservoirs comprises different reagents, optionally wherein each of the plurality of reagent reservoirs comprises different reagents, and/or optionally wherein two of the plurality of reagent reservoirs comprise an identical reagent.

20. The microfluidic chip of any one of claims 1-19, wherein a cross-sectional shape of the product reservoir and a cross-sectional shape of the waste reservoir are identical.

21. The microfluidic chip of any one of claims 1-19, wherein a cross-sectional shape of the product reservoir and a cross-sectional shape of the waste reservoir are different.

22. The microfluidic chip of any one of claims 1-21,
wherein a cross-sectional shape of the product reservoir is a rectangle, a circle, an ellipse, a semicircle, a trapezoid, or a combination thereof,
wherein a cross-sectional shape of the waste reservoir is a rectangle, a circle, an ellipse a semicircle, a trapezoid, or a combination thereof,
wherein a cross-sectional shape of one, one or more, of each of the plurality of reagent reservoirs is a circle, a rectangle, an ellipse, a semicircle, a trapezoid, or a combination thereof,
wherein a cross-sectional shape of the reagent exchange reservoir is a circle, a rectangle, an ellipse, a semicircle, a trapezoid, or a combination thereof, and/or
wherein a cross-sectional shape of the reaction chamber is a circle, a rectangle, an ellipse, a semicircle, a trapezoid, or a combination thereof.

23. The microfluidic device of any one of claims 12-22, wherein a size of the product reservoir, a size of the waste reservoir, a size of one, one or more, of each of the plurality of reagent reservoirs, a size of the reagent exchange reservoir, a size of the reaction chamber, a size of the microfluidic device, a size of the reagent exchange unit, and/or a size of the reaction unit is 1 mm to 20 cm.

24. The microfluidic device of any one of claims 12-23, wherein a cross-sectional shape of one, one or more, or each of the plurality of fluid microchannels is a circle, a rectangle, an ellipse, a semicircle, a trapezoid, or a combination thereof, and/or wherein a size of one, one or more, or each of the plurality of fluid microchannels is 1 mm to 20 cm.

25. The microfluidic chip of any one of claims 1-24, wherein a shape of the microfluidic device, a shape of the reagent exchange unit, a shape of the reaction unit is a circle, a rectangle, an ellipse, a semicircle, a trapezoid, or a combination thereof, and/or a size of the microfluidic device, a size of the reagent exchange unit, a size of the reaction unit is 1 cm to 30 cm.

26. The microfluidic device of any one of claims 1-25, wherein the reaction chamber comprises two tapered ends forming the inlet and the outlet of the reaction chamber.

27. The microfluidic device of any one of claims 1-26, wherein the reaction chamber comprises a microwell array comprising at least 100 microwells, optionally wherein the microwell array is disposed on the upper surface of the reaction unit.

28. The microfluidic device of any one of claims 1-27, wherein the lower surface of the reaction unit is capable of being in thermal contact with a heating element.

29. The microfluidic device of any one of claims 12-28, wherein the plurality of microchannels and/or the reaction chamber is in a recess of the lower surface of the reagent exchange unit, and/or wherein the reaction unit covers the plurality of microchannels, the reaction chamber, and the recess, and forms, together with the recess and/or the lower surface of the reagent exchange unit, the plurality of microchannels and the reaction chamber of the microfluidic device.

30. The microfluidic device of any one of claims 12-29, wherein the reagent exchange unit and/or the reaction unit comprises (i) the reaction chamber, or a portion thereof, and/or (ii) the plurality of fluid microchannels, or a portion of each of one or more fluid microchannels of the plurality of fluid microchannels.

31. The microfluidic device of any one of claims 12-30, wherein the lower surface of the reagent exchange unit and/or the upper surface of the reaction unit comprises (i) the reaction chamber, or a portion thereof, and/or (ii) the plurality of fluid microchannels, or a portion of each of one or more fluid microchannels of the plurality of fluid microchannels.

32. A gas-flow control device comprising:
a plate;
a plurality of gas injection valves disposed on and in the plate; and
a plurality of gas injection microchannels disposed in the plate, each having an outlet open end on a lower surface of the plate and each connected with one injection valve of the plurality of injection valves.

33. The gas-flow control device of claim 32, wherein the plurality of gas injection valves comprises a plurality of reagent gas injection valves, and wherein the plurality of gas injection microchannels comprises a plurality of reagent gas injection microchannels.

34. The gas-flow control device of any one of claims 32-33, further comprising:
a plurality of gas extraction valves disposed on and in the plate; and
a plurality of gas extraction microchannels disposed in the plate, each having an inlet open end on the lower surface of the plate, wherein each of the plurality of gas extraction microchannels is connected with a gas extraction vale of the plurality of gas extraction valves.

35. The gas-flow control device of claim 34,
wherein the plurality of gas extraction valves comprises a product gas extraction valve and/or a waste gas extraction valve, and
wherein the plurality of gas extraction microchannels comprises a product gas extraction microchannel and/or a waste gas extraction microchannel,
wherein the product gas extraction microchannel is connected with the product gas extraction valve, and/or
wherein the waste gas extraction microchannel is connected with the waste gas extraction valve.

36. The gas-flow control device of any one of claims 32-35,
wherein the plurality of gas extraction valves comprises a reagent exchange gas extraction valve, and wherein the plurality of gas extraction microchannels comprises a reagent exchange gas extraction microchannel, and
wherein the plurality of gas injection valves comprises a reagent exchange gas injection valve, and wherein the plurality of gas injection microchannels comprises a reagent exchange gas injection microchannel.

37. The gas-flow control device of any one of claims 32-35,
wherein the plurality of gas extraction valves comprises a mixing gas extraction valve, and wherein the plurality of gas extraction microchannels comprises a mixing gas extraction microchannel, and
wherein the plurality of gas injection valves comprises a mixing gas injection valve, and wherein the plurality of gas injection microchannels comprises a mixing gas injection microchannel.

38. A gas-flow control device comprising:
a plurality of gas injection valves and a plurality of gas extraction valves disposed on and in a plate of the gas-flow device;
a plurality of gas injection microchannels disposed in the plate, each having an outlet open end on a lower surface of the plate and an inlet open end connected with one injection valve of the plurality of injection valves; and
a plurality of gas extraction microchannels disposed in the plate, each having an inlet open end on the lower surface of the plate, wherein an outlet open end of a waste gas extraction microchannel and an outlet open end of a product gas extraction microchannel are connected to a waste gas extraction valve and a product gas extraction valve, respectively, of the plurality of gas extraction valves.

39. A gas-flow control device comprising:
a plurality of gas injection valves and a plurality of gas extraction valves disposed on and in a plate of the gas-flow device;
a plurality of gas injection microchannels disposed in the plate, each having an outlet open end on a lower surface of the plate and an inlet open end connected with one injection valve of the plurality of injection valves; and
a plurality of gas extraction microchannels disposed in the plate, each having an inlet open end on the lower surface of the plate and an outlet open end connected to a gas extraction valve of the plurality of gas extraction valves.

40. A gas-flow control device comprising:
a plurality of gas injection microchannels disposed in a plate of the gas-flow control device, each having an outlet open end on a lower surface of the plate and an inlet open end for connecting to one injection valve of a plurality of injection valves, and
a plurality of gas extraction microchannels disposed in the plate, each having an inlet open end on the lower surface of the plate and an outlet open end for connecting to a gas extraction valve of a plurality of gas extraction valves.

41. A gas-flow control device comprising:
a plurality of gas injection microchannels disposed in a plate of the gas-flow control device, each having an outlet open end on a lower surface of the plate and an inlet open end disposed within the plate; and
a plurality of gas extraction microchannels disposed in the plate, each having an inlet open end on the lower surface of the plate and an outlet open end for connecting to a gas extraction valve disposed within the plate.

42. The gas-flow control device of any one of claims 40-41, further comprising a plurality of gas injection valves and a plurality of gas extraction valves disposed on and in the plate, wherein the inlet open end of each of the plurality of gas injection microchannels is connected to a gas injection valve of the plurality of gas injection valves, and wherein the outlet open end of each of the plurality of gas extraction microchannels is connected to a gas extraction valve of the plurality of gas extraction valves.

43. The gas-flow control device of any one of claims 32-42, wherein a majority of, or all of, the plurality of gas injection valves and/or a majority of, or all of, the plurality of gas extraction valves are arranged on one end of the plate.

44. The gas-flow control device of any one of claims 32-43, wherein one or more of the plurality of injection valves is not connected to a gas injection microchannel of the plurality of gas injection microchannels, and/or one or more of the plurality of extraction valves is not connected to a gas extraction microchannel of the plurality of gas extraction microchannels.

45. The gas-flow control device of any one of claims 32-44, wherein one, one or more, or each of the plurality of gas injection valves when pressurized with a driving gas and in an open state injects the driving gas in a direction from the inlet open end of a gas injection microchannel of the plurality of gas injection microchannels to the outlet open end of the gas injection microchannel, and/or wherein one, one or more, or each of the plurality of gas extraction valves under suction and in an open state allows a gas to flow in a direction from the inlet open end of a gas extraction microchannel of the plurality of gas injection microchannels to the outlet open end of the gas extraction microchannel.

46. The gas-flow control device of any one of claims 32-45, wherein a gas injection valve of the plurality of gas injection valves controls an amount of gas exiting the outlet open end of the corresponding gas injection microchannel, and/or wherein a gas extraction valve of the plurality of gas extraction valves controls an amount of gas entering the inlet open end of the corresponding gas extraction microchannel.

47. The gas-flow control device of any one of claims 32-46, wherein one, one or more, or each of the plurality of gas injection valves is a solenoid valve, and/or wherein one, one or more, or each of the plurality of gas extraction valves is a solenoid valve.

48. The gas-flow control device of any one of claims 32-47, wherein the plate further comprises an observation window.

49. The gas-flow control device of any one of claims 32-48,
wherein a size of one, one or more, or each of the plurality gas injection microchannels is 1 mm to 20 cm,
wherein a size of the inlet and/or the outlet of one, one or more, or each of the plurality gas injection microchannels is 0.1 mm to 5 mm,
wherein a size of one, one or more, or each of the plurality gas extraction microchannels is 1 mm to 20 cm,
wherein a size of the inlet and/or the outlet of one, one or more, or each of the plurality gas extraction microchannels is 0.1 mm to 5 mm, and/or
wherein a size of the gas-flow control device is 5 mm to 40 cm.

50. The gas-flow control device of any one of claims 32-49, wherein a cross-sectional shape of one, one or more, or each of the plurality of gas injection microchannels is a circle, a rectangle, an ellipse, a semicircle, a trapezoid, or a combination thereof, and/or a cross-sectional shape of one, one or more, or each of the plurality of gas extraction microchannels is a circle, a rectangle, an ellipse, a semicircle, a trapezoid, or a combination thereof.

51. The gas-flow control device of any one of claims 32-50,
wherein the plate comprises a plurality of layers, wherein each of the plurality of layers is reversibly coupled to at least one other layer of the plurality of layers,
wherein one or more gas injection valves of the plurality of gas injection valves and/or one or more gas extraction valves of the plurality gas extraction valves are disposed on and through a first layer of the plurality of layers,
where the first layer comprises a plurality of grooves, wherein a second layer of the plurality of layers cover the plurality of grooves to form the plurality of gas injection microchannels and/or the plurality of gas extraction microchannels,
wherein the one or more gas injection valves and the one or more gas extraction valves are disposed in or through a second layer of the plurality of layers, optionally wherein one or more gas injection microchannels of the plurality of gas injection microchannels and/or one or more gas extraction microchannels of the plurality of gas extraction microchannels are formed between and/or by the first layer and the second layer, optionally wherein the second layer is a cover layer, and/or
wherein the plurality of layers comprises a third layer that is a cover layer.

52. A reaction module comprising:
a microfluidic device of any one of claims 1-31; and
a gas-flow control device of any one of claims 32-51 capable of detachably coupling to and/or forming an air tight seal with the microfluidic device, or one or more reservoirs thereof.

53. A reaction module comprising:
a microfluidic device of any one of claims 1-31; and
a gas-flow control device of any one of claims 32-51, wherein an area on a surface of the gas-flow control device surrounding the outlet open end of one gas injection microchannel of plurality of gas injection microchannels is capable of detachably coupling to and/or forming an air tight seal with one reagent reservoir of the plurality of reagent reservoirs, wherein an area on the surface of the gas-flow control device surrounding the inlet open end of the waste gas extraction microchannel is capable of detachably coupling to and/or forming an air tight seal with the waste reservoir to result, and wherein an area on the surface of the gas-flow control device surrounding the inlet open end of the product gas extraction microchannel is capable of detachably coupling to and/or forming an air tight seal with the product reservoir.

54. A reaction module comprising:
a microfluidic device of any one of claims 12-31; and
a gas-flow control device of any one of claims 32-51, wherein the gas-flow control device is capable of detachably coupling to and/or forming an air tight seal with one reagent reservoir of the plurality of reagent reservoirs to result in a space comprising the outlet open end of a gas injection microchannel of plurality of gas injection microchannels, wherein the gas-flow control device is capable of detachably coupling to and/or forming an air tight seal with the waste reservoir to result in a space comprising the inlet open end of the waste gas extraction microchannel, and wherein the gas-flow control device is capable of detachably coupling to and/or forming an air tight seal with the product reservoir to result in a space comprising the inlet open end of the product gas extraction microchannel.

55. The reaction module of any one of claims 52-54, wherein the gas-flow control device is attached to and/or forms an air tight seal with the microfluidic device, or a portion thereof, optionally via a silicone pad sandwiched between the gas-flow control device and the microfluidic device, optionally wherein the silicon pad comprises a plurality of through holes allowing gaseous communication of the outlet opening ends of the gas injection microchannels with the reagent reservoirs and the inlet opening ends of the gas extraction microchannels with the waste reservoir and the product reservoir, optionally wherein the silicon pad comprises a plurality of through holes at positions, when aligned with and sandwiched between the gas-flow control device and the microfluidic device, corresponding to the positions of the outlet opening ends of the gas injection microchannels and the inlet opening ends of the gas extraction microchannels.

56. The reaction module of claim 55,
wherein one, one or more, or each of the plurality of gas injection microchannels is in gaseous communication with one of the plurality of reagent reservoirs,
wherein the outlet open end of one, one or more, or each of the plurality of gas injection microchannels is open to one of the plurality of reagent reservoirs,
wherein the waste gas extraction microchannel is in gaseous communication with the waste reservoir,
wherein the inlet open end of the waste gas extraction microchannel is open to the waste reservoir,
wherein the product gas extraction microchannel is in gaseous communication with the product reservoir,
wherein the inlet open end of the product gas extraction microchannel is open to the product reservoir, and/or
wherein the reagent exchange gas injection microchannel is in gaseous communication with the reagent exchange reservoir, wherein the outlet open end of the reagent exchange gas injection microchannel is open to the reagent exchange reservoir, wherein the reagent exchange gas extraction microchannel is in gaseous communication with the reagent exchange reservoir, and/or wherein the inlet open end of the reagent exchange gas extraction microchannel is open to the reagent exchange reservoir.

57. The reaction module of claim 56,
wherein when a driving gas exits the outlet of the gas injection microchannel into the reagent reservoir, a reagent in the reagent reservoir is driven from the reagent reservoir through a fluid microchannel of the plurality of fluid microchannels into the reagent exchange reservoir, wherein when a gas exits the inlet of the waste gas extraction microchannel, one or more reagents in the reagent exchange reservoir are pulled from the reagent exchange reservoir into the reaction chamber then into the waste reservoir, and/or wherein when a gas exits the inlet of the product gas extraction microchannel, one or more reagents in the reagent exchange reservoir are pulled from the reagent exchange reservoir into the reaction chamber then into the product reservoir
wherein when a driving gas exits the outlet of the gas injection microchannel into the reagent reservoir, (i) a reagent in the reagent reservoir is driven from the reagent reservoir through a fluid microchannel of the plurality of fluid microchannels into the reagent exchange reservoir, and (ii) a gas in the reagent exchange reservoir exits the reagent exchange reservoir, wherein when a driving gas exits the outlet of the reagent exchange gas injection microchannel into the reagent exchange reservoir, one or more reagents in the reagent exchange reservoir are driven from the reagent exchange reservoir into the reaction chamber, wherein when a gas exits the inlet of the waste gas extraction microchannel, one or more reagents in the reagent exchange reservoir are pulled from the reagent exchange reservoir into the reaction chamber then into the waste reservoir, and/or wherein when a gas exits the inlet of the product gas extraction microchannel, one or more reagents in the reagent exchange reservoir are pulled from the reagent exchange reservoir into the reaction chamber then into the product reservoir, optionally wherein the one or more reagents in the reagent exchange reservoir are mixed in the reagent exchange reservoir,
wherein when a gas exits the inlet of the waste gas extraction microchannel from the waste reservoir, a waste in the reaction chamber is pulled from the reaction chamber through the waste fluid microchannel into the waste reservoir, and/or
wherein when a gas exits the inlet of the product gas extraction microchannel from the product reservoir, a product in the reaction chamber is pulled from the reaction chamber through the product fluid microchannel into the product reservoir, optionally wherein the product is generated using at least one reagent.

58. The reaction module of claim 56,
(a) wherein when the gas injection microchannel is under a positive pressure and/or the gas injection valve is in an open state and/or (ii) the waste gas extraction microchannel is under a negative pressure and/or the waste gas extraction valve is in an open state, (1) a reagent in the reagent reservoir is driven through a fluid microchannel of the plurality of fluid microchannels into the reagent exchange reservoir then into the reaction chamber, and/or (2) a waste generated in the reaction chamber from the reagent is driven from the reaction chamber through the waste fluid microchannel into the waste reservoir,
(b) wherein when the gas injection microchannel is under a positive pressure and/or the gas injection valve is in an open state and/or when) the product gas extraction microchannel is under a negative pressure and/or the product gas extraction valve is in an open state, a reagent in the reagent reservoir is driven through a fluid microchannel of the plurality of fluid microchannels into the reagent exchange reservoir then into the reaction chamber, and a product generated in the reaction chamber from the reagent is driven from the reaction chamber through the product fluid microchannel into the product reservoir,
(c) wherein when the gas injection microchannel is under a positive pressure and/or the gas injection valve is in an open state, a reagent in the reagent reservoir is driven through a fluid microchannel of the plurality of fluid microchannels into the reagent exchange reservoir,
wherein when the waste gas extraction microchannel is under a negative pressure and/or the waste gas extraction valve is in an open state, (1) a reagent in the reagent exchange reservoir is pulled into the reaction chamber, and (2) a waste generated in the reaction chamber from the reagent is driven from the reaction chamber through the waste fluid microchannel into the waste reservoir, and
wherein when the product gas extraction microchannel is under a negative pressure and/or the product gas extraction valve is in an open state, (1) a reagent in the reagent exchange reservoir is pulled into the reaction chamber, and (2) a product generated in the reaction chamber from the reagent is driven from the reaction chamber through the product fluid microchannel into the product reservoir, optionally wherein the product is generated using the reagent, and/or
(d) wherein when the mixing gas extraction microchannel is under a negative pressure and/or the mixing gas extraction valve is in an open state, two or more reagents in the reagent exchange reservoir are pulled from the reagent exchange reservoir into the mixing reservoir, thereby mixing the two or more reagents,
wherein when the mixing gas injection microchannel is under a positive pressure and/or the mixing gas injection valve is in an open state, the one or more reagents in the mixing reservoir are driven into the reagent exchange reservoir,
wherein when the waste gas extraction microchannel is under a negative pressure and/or the waste gas extraction valve is in an open state, the one or more reagents in the reagent exchange reservoir are pulled from the reagent exchange reservoir into the reaction chamber wherein a waste is generated in the reaction chamber from the one or more reagents and the waste is pulled from the reaction chamber through the waste fluid microchannel into the waste reservoir, and
wherein when the product gas extraction microchannel is under a negative pressure and/or the product gas extraction valve is in an open state, the one or more reagents in the reagent exchange reservoir are pulled from the reagent exchange reservoir into the reaction chamber where a product is generated using the one or more reagents and the product is pulled into the product reservoir.

59. A sample preparation device comprising:
a reaction module of any one of claims 52-58; and
a heating element in contact with the microfluidic device of the reaction module.

60. The sample preparation device of claim 59, wherein the microfluidic device is sandwiched between the gas-flow control device and the heating element.

61. A sample preparation device comprising:
a gas-flow control device of any one of claims 32-51 capable of detachably coupling to and/or forming an air tight seal with a microfluidic device of any one of claims 12-31; and
a heating element for heating the microfluidic device.

62. The sample preparation device of claim 61, wherein the microfluidic device is sandwiched between the gas-flow control device and the heating element when the microfluidic device, the gas-flow control device, and the heating element are in an assembled state, optionally wherein the microfluidic device is below the gas-flow control device in the assembled state, optionally wherein the heating element is below the microfluidic device in the assembled state.

63. The sample preparation device of any one of claims 59-62, further comprising an injection pump for providing a gas to the plurality of gas injection valves and/or an extraction pump for providing a suction to the plurality of gas extracting valves, optionally wherein the injection pump is the extraction pump, optionally wherein the injection pump and/or the extraction pump is adjacent the reaction module and/or below the reaction module when the sample preparation device is in an upright orientation.

64. The sample preparation device of any one of claims 59-63, further comprising a control unit in electrical communication with and/or controls the plurality of gas injection valves, the plurality of gas extraction valves, the heating element, the injection pump, and/or the extraction pump, optionally wherein the control unit is adjacent the reaction module and/or below the reaction module when the sample preparation device is in an upright orientation, optionally wherein the control unit is adjacent the injection pump and/or the extraction pump.

65. The sample preparation device of any one of claims 59-64, further comprising a housing to which the gas-flow control device, the heating element, the control unit, the injection pump, and/or the extraction pump are attached.

66. The sample preparation device of any one of claims 59-65, wherein a size of the sample preparation device is 10 mm to 100 cm.

67. A sample preparation system comprising:
at least one gas-flow control device of any one of claims 32-51; and
at least one drive module capable of detachably coupling to a microfluidic device of any one of claims 12-31 to and/or the gas-flow control device.

68. The sample preparation system of claim 67, wherein the at least one drive module comprises:
a microfluidic device drive module for moving the microfluidic device, optionally wherein the microfluidic device drive module is for moving the microfluidic device horizontally between an away horizontal position and a coupling horizontal position, optionally wherein when the microfluidic device drive module is in the away horizontal position, the microfluidic device is not below the gas-flow control module, and optionally wherein when the microfluidic device drive module is in the coupling horizontal position, the microfluidic device is below the gas-flow control device or is detachably coupled to and/or forms an air tight seal with the gas-flow control device, optionally wherein the microfluidic device drive module comprises at least one sliding table assembly, optionally wherein the sliding table assembly comprises a sliding table, a sliding table support base, and a stepping motor; and
a gas-flow control drive module for moving the gas-flow control device, optionally wherein the gas-flow control drive module is for moving the gas-flow control module vertically between an away vertical position and a coupling vertical position, optionally wherein when the microfluidic device drive module is in the coupling horizontal position and the gas-flow control drive module is in the away vertical position, the microfluidic device is below the gas-flow control device, optionally wherein when the microfluidic device drive module is in the coupling horizontal position and the gas-flow control drive module is in the coupling vertical position, the microfluidic device is detachably coupled to and/or forms an air tight seal with the microfluidic device, optionally wherein the gas-flow control drive module comprises at least one push-rod assembly, optionally wherein the push-rod assembly comprises a drive motor, a gear shaft attached to the drive motor, a slide rail, and a gear rack.

69. The sample preparation system of any one of claims 67-68, further comprising a heating element for heating the microfluidic device, optionally the heating element is for heating the microfluidic device from below.

70. The sample preparation system of any one of claims 67-69, further comprising an injection pump for providing a gas to the plurality of gas injection valves and/or an extraction pump for providing a suction to the gas extracting valves, optionally wherein the injection pump is the extraction pump.

71. The sample preparation system of any one of claims 67-70, further comprising a control unit, wherein the control unit is in electrical communication and/or controls the plurality of gas injection valves, the plurality of gas extraction valves, the heating element, the injection pump, the extraction pump, the drive module, the horizontal drive module, and/or the vertical drive module.

72. The sample preparation system of any one of claims 67-71, further comprising a housing, wherein the gas-flow control device, the heating element, the control unit, the injection pump, the extraction pump, the at least one drive module, the microfluidic device drive module, and/or the gas-flow control device drive module are attached and/or fixed to the housing.

73. The sample preparation system of any one of claims 71-72, wherein the control unit comprises a control unit interface for controlling and/or programming the control unit using a computer, a control software, a programmable software, or a combination thereof, optionally wherein the sample preparation system comprises the computer.

74. A method of performing a reaction using a microfluidic device of any one of claims 12-31, a gas-flow control device of any one of claims 32-51, a reaction module of any one of claims 52-58, a sample preparation device of 59-66, and/or the sample preparation system of any one of claims 67-72.

75. A method of reagent loading comprising:
(a) providing the microfluidic device according to any one of claims 1-31;
(b) loading a first reagent a first reagent reservoir of the plurality of reagent reservoirs; and
(c1) flowing the first reagent from the first reagent reservoir into the reagent exchange reservoir through a first fluid microchannel of the plurality fluid microchannels, then into the reaction chamber, and then into the waste reservoir.

76. A method of reagent loading comprising:
(a) providing the microfluidic device according to any one of claims 1-31;
(b) loading a first reagent and a second reagent into a first reagent reservoir and a second reagent reservoir of the plurality of reagent reservoirs;
(c1) flowing the first reagent from the first reagent reservoir into the reagent exchange reservoir through a first fluid microchannel of the plurality fluid microchannels, then into the reaction chamber, and then into the waste reservoir; and
(c2) flowing the second reagent from the second reagent reservoir into the reagent exchange reservoir chamber through a second fluid microchannel of the plurality fluid microchannels, then into the reaction chamber, and then into the waste reservoir.

77. The method of claim 76, further comprising:
(b2) loading a third reagent into a third reagent reservoir of the plurality of reagent reservoirs;
(c3) flowing the third reagent into the into the reagent exchange reservoir through a third fluid microchannel of the plurality fluid microchannels then into the reaction chamber, thereby a reaction occurs in the reaction chamber; and
(d) flowing one or more waste products generated in the reaction chamber into the waste reservoir, and/or flowing one or more reaction products in the reaction chamber into the product reservoir.

78. A method of reagent loading comprising:
(a) providing the microfluidic device according to any one of claims 1-31, wherein each of the plurality of reagent reservoirs comprises a reagent;
(c) sequentially flowing the reagent in each of the plurality of reagent reservoirs into the reagent exchange reservoir through a fluid microchannel of the plurality fluid microchannels and then into the reaction chamber; and
(d) flowing one or more reaction products in the reaction chamber into the product reservoir.

79. The method of any one of claims 76-77, wherein the first reagent comprises a plurality of cells, wherein the second reagent comprises a plurality of particles, wherein one, one or more, or each of the plurality of particles comprises a plurality of barcode molecules, thereby single cells and single particles are loaded into microwells of the microwell array.

80. The method of any one of claims 76-79, wherein the third reagent comprises a cell lysis reagent, an enzyme, PCR primers, and/or therapeutic compounds, and/or wherein the reaction products comprise a plurality of barcoded target nucleic acids and/or reverse transcription products.

81. The method of any one of claims 76-80, wherein the reaction comprises cell lysis, ligand-binding, cell-cell interaction, cell capture, nucleic acid synthesis, cellular response to a therapeutic compound, nucleic acid barcoding, reverse transcription, or a combination thereof.

82. The method of any one of claims 76-81, wherein the microfluidic device is reversibly coupled to a gas-flow control device of any one of claims 32-51, wherein flowing the reagents comprises flowing the reagents using one or more gas injection valves of the plurality of gas injection valves and one or more gas extraction valves of the plurality of gas extraction valves, optionally wherein the gas-flow control device is comprised in a reaction module of any one of claims 52-58, a sample preparation device of any one of claims 59-66, and/or the sample preparation system of any one of claims 67-72, optionally wherein flowing the reagents comprises controlling the gas injection valves and gas extraction valves using the control unit to flow the reagents.

83. A method of nucleic acid analysis, comprising:
generating a plurality of barcoded target nucleic acids using a method of any one of claims 80-82; and
analyzing the plurality of barcoded target nucleic acids.

84. The method of claim 83, wherein analyzing the plurality of barcoded target nucleic acids comprises determining the sequences of the plurality of barcoded target nucleic acids.

85. A method of performing a reaction comprising:
(a1) providing a microfluidic device of any one of claims 12-31 and a gas-flow control device of any one of claims 32-51 and reversibly coupling the microfluidic device and the gas-flow control device, or providing a reaction module of any one of claims 52-58
(b) loading one or more reagents into the plurality of reagent reservoirs;
for each reagent reservoir of the plurality of reagent reservoirs loaded with the one or more reagents;
(c) injecting gas into the reagent reservoir through a gas injection valve of the plurality of gas injection valves and a gas injection microchannel of the plurality of gas injection microchannels, thereby applying a positive gas pressure to the reagent reservoir, thereby injecting the one or more reagents from the reagent reservoir into the reagent exchange reservoir; and
(d1) extracting gas from the waste reservoir through the waste gas extraction valve and the waste gas extraction microchannel, thereby applying a negative gas pressure to the waste reservoir, thereby transferring the one or more reagents from the reagent exchange reservoir to the reaction chamber; and/or
(d2) extracting gas from the product reservoir through the product gas extraction valve and the product gas extraction microchannel, thereby applying a negative gas pressure to the product reservoir, thereby transferring the one or more reagents from the reagent exchange reservoir to the reaction chamber;
(e) allowing the one or more reagents to react in the reaction chamber;
(f1) extracting gas from the waste reservoir through the waste gas extraction valve and the waste gas extraction microchannel, thereby applying a negative gas pressure to the waste reservoir, thereby extracting a waste from the reaction chamber into the waste reservoir; and
(f2) extracting gas from the product reservoir through the product gas extraction valve and the product gas extraction microchannel, thereby applying a negative gas pressure to the product reservoir, thereby extracting a product from the reaction chamber into the product reservoir.

86. A method of performing a reaction comprising:
(a1) providing the sample preparation device of any one of claims 59-66, or the sample preparation system of any one of claims 67-72 and a microfluidic device of any one of claims 12-31;
(a2) coupling each of the one or more gas-flow control devices to a microfluidic device of the one or more microfluidic devices;
(b) loading one or more reagents into the plurality of reagent reservoirs;
for each reagent reservoir of the plurality of reagent reservoirs loaded with the one or more reagents;
(c) injecting gas into the reagent reservoir through a gas injection valve of the plurality of gas injection valves and a gas injection microchannel of the plurality of gas injection microchannels, thereby applying a positive gas pressure to the reagent reservoir, thereby injecting the one or more reagents from the reagent reservoir into the reagent exchange reservoir;
(d1) extracting gas from the waste reservoir through the waste gas extraction valve and the waste gas extraction microchannel, thereby applying a negative gas pressure to the waste reservoir, thereby transferring the one or more reagents from the reagent exchange reservoir to the reaction chamber; and/or
(d2) extracting gas from the product reservoir through the product gas extraction valve and the product gas extraction microchannel, thereby applying a negative gas pressure to the product reservoir, thereby transferring the one or more reagents from the reagent exchange reservoir to the reaction chamber;
(e) allowing the one or more reagents to react in the reaction chamber;
(f1) extracting gas from the waste reservoir through the waste gas extraction valve and the waste gas extraction microchannel, thereby applying a negative gas pressure to the waste reservoir, thereby extracting a waste from the reaction chamber into the waste reservoir; and
(f2) extracting gas from the product reservoir through the product gas extraction valve and the product gas extraction microchannel, thereby applying a negative gas pressure to the product reservoir, thereby extracting a product from the reaction chamber into the product reservoir.

87. The method of claim 86, wherein the coupling of (a2) further comprises moving the gas-flow control module and/or moving the reaction module, thereby aligning the gas-flow control module and the reaction module.
